(19) United States
(12) Patent Application Publication
(10) Pub. No.: US 2026/0243771 A1
(43) Pub. Date: Aug. 20, 2026

(54) DIAGNOSTIC KITS AND METHODS FOR EARLY DETECTION OF OVARIAN CANCER

(71) Applicants: RAMOT AT TEL-AVIV UNIVERSITY LTD., Tel Aviv (IL); SHEBA IMPACT LTD., Ramat Gan (IL)

(72) Inventors: Tamar GEIGER, Tel Aviv (IL); Keren LEVANON, Ramat Gan (IL); Georgina D. BARNABAS, Tel Aviv (IL); Keren BAHAR-SHANY, Ramat Gan (IL); Jacob KORACH, Ramat Gan (IL)

(73) Assignees: RAMOT AT TEL-AVIV UNIVERSITY LTD., Tel Aviv (IL); SHEBA IMPACT LTD., Ramat Gan (IL)

(21) Appl. No.: 18/880,897

(22) PCT Filed: Jul. 5, 2023
(86) PCT No.: PCT/IL2023/050694

§ 371 Date: Jan. 3, 2025

Related U.S. Application Data
(60) Provisional application No. 63/367,698, filed on Jul. 5, 2022.

(51) Int. Cl. G01N 33/57545

(52) U.S. Cl. CPC G01N 33/57545; G01N 2800/7028

(57) ABSTRACT

The present disclosure provides diagnostic methods, compositions and kits for detecting ovarian cancer in a subject, specifically, High grade ovarian cancer, more specifically in genetically predisposed patients. In some embodiments, the diagnostic methods, compositions and kits are based on at least two biomarker proteins of a specific 7-protein signature.

### FIELD OF THE INVENTION

The invention relates to early diagnosis of cancer. More specifically, the present disclosure provides methods, kits and compositions based on biomarker signature, for early diagnosis of ovarian cancer.

### BACKGROUND ART

References considered to be relevant as background to the presently disclosed subject matter are listed below:
[1] Buys S S, Partridge E, Black A, Johnson C C, Lamerato L, Isaacs C, et al. Effect of screening on ovarian cancer mortality: the Prostate, Lung, Colorectal and Ovarian (PLCO) Cancer Screening Randomized Controlled Trial. JAMA. 2011; 305(22):2295-303.
[2] Freydanck M K, Laubender R P, Rack B, Schuhmacher L, Jeschke U, Scholz C. Two-marker combinations for preoperative discrimination of benign and malignant ovarian masses. Anticancer Res. 2012 May; 32(5):2003-8.
[3] Stukan M, Dudziak M, Ratajczak K, Grabowski J P. Usefulness of diagnostic indices comprising clinical, sonographic, and biomarker data for discriminating benign from malignant ovarian masses. J Ultrasound Med. 2015 February; 34(2):207-17.
[4] Jacobs I J, Menon U, Ryan A, Gentry-Maharaj A, Burnell M, Kalsi J K, et al. Ovarian cancer screening and mortality in the UK Collaborative Trial of Ovarian Cancer Screening (UKCTOCS): a randomised controlled trial. Lancet. 2015 December;
[5] Rebbeck T R, Kauff N D, Domchek S M. Meta-analysis of risk reduction estimates associated with risk-reducing salpingo-oophorectomy in BRCA1 or BRCA2 mutation carriers. J Natl Cancer Inst. 2009 Jan. 21; 101(2):80-7.
[6] Reade C J, McVey R M, Tone A A, Finlayson S J, McAlpine J N, Fung-Kee-Fung M, et al. The fallopian tube as the origin of high grade serous ovarian cancer: review of a paradigm shift. J Obstet Gynaecol Can. 2014 March; 36(2):133-40.
[7] Dubeau L. The cell of origin of ovarian epithelial tumours. Lancet Oncol. 2008; 9(12):1191-7.
[8] Levanon K, Crum C, Drapkin R. New insights into the pathogenesis of serous ovarian cancer and its clinical impact. J Clin Oncol. 2008; 26(32):5284-93.
[9] Erickson B K, Kinde I, Dobbin Z C, Wang Y, Martin J Y, Alvarez R D, et al. Detection of somatic TP53 mutations in tampons of patients with high-grade serous ovarian cancer. Obstet Gynecol. 2014 November; 124(5):881-5.
[10] Kinde I, Bettegowda C, Wang Y, Wu J, Agrawal N, Shih Ie M, et al. Evaluation of DNA from the Papanicolaou test to detect ovarian and endometrial cancers. Sci Transl Med. 2013; 5(167):167ra4.
[11] Maritschnegg E, Wang Y, Pecha N, Horvat R, Van Nieuwenhuysen E, Vergote I, et al. Lavage of the Uterine Cavity for Molecular Detection of Müllerian Duct Carcinomas: A Proof-of-Concept Study. J Clin Oncol. 2015 Nov. 9; 33(36):4293-300.
[12] Krimmel J D, Schmitt M W, Harrell M I, Agnew K J, Kennedy S R, Emond M J, et al. Ultra-deep sequencing detects ovarian cancer cells in peritoneal fluid and reveals somatic TP53 mutations in noncancer ous tissues. Proc Natl Acad Sci USA. 2016 May 24; 113(21):6005-10.
[13] ClinicalTrials.gov identifier: NCT03150121, PI: Dr. Keren Levanon;
[14] Barnabas G. D. et al., Molecular & Cellular Proteomics 18:865-875 (2019);
[15] WO 2018/178993.
[16] Brouwer J, Kluiver J, de Almeida R, Modderman R, Terpstra M, Kok K, et al. Small RNA sequencing reveals a comprehensive miRNA signature of BRCA1-associated high-grade serous ovarian cancer. J Clin Pathol. 2016 Nov. 1; 69(11):979-85.

Acknowledgement of the above references herein is not to be inferred as meaning that these are in any way relevant to the patentability of the presently disclosed subject matter.

### BACKGROUND OF THE INVENTION

Overall survival of high grade epithelial ovarian cancer (OVCA) patients correlates with disease stage at diagnosis: while patients with stage I disease have >90% 5-year overall survival, rates for stage IV disease are extremely low. Regrettably OVCA is diagnosed at an advanced stage in ~75% of the cases regardless of adherence to testing recommendations (1). This grim reality stems primarily from the lack of effective screening programs and lack of early stage-specific biomarkers. A multitude of biomarkers have been proposed and tested over the years, but none have been shown to improve survival. The most widely used plasma-based biomarkers are CA125 and HE4, either individually or in combination (the latter with 76% sensitivity and 95% specificity) (2), or the combination of these biomarkers with clinical and imaging parameters (3). The large UK Collaborative Trial of Ovarian Cancer Screening (UKCTOCS) study showed a very modest effect on survival with implementation of a complicated blood CA125-based monitoring algorithm (4). Additionally, a large-scale prostate, lung, colon and ovarian cancer (PLCO) screening study failed to show reduced ovarian cancer-related mortality in intermediate risk women who were screened using semiannual plasma CA125 levels and transvaginal ultrasound (1). These blood-detectable proteins correlate with tumor volume, and hence fail to diagnose the earliest, potentially curable lesions before they have disseminated intra-peritoneally or even to distant organs.

The lack of a validated monitoring scheme, combined with the considerably high risk attributed to specific recognized susceptibility loci, including: BRCA1/2, BARD1, BRIP1, CHEK2, MRE11A, MSH6, NBN, PALB2, RAD50, RAD51C, RAD51D, TP53, APC, ATM, DICER1, MUTYH [Eoh K J, et al. Cancer Res Treat. 2018 July; 50(3):917-925], result in the unequivocal recommendation for women at high risk to undergo risk-reducing bilateral salpingo-oophorectomy (RRBSO) typically at age 35-40 years. This surgery reduces the risk for development of OVCA by ~85% (5). Although RRBSO has been proven to reduce mortality significantly (5), early surgical menopause and its related health consequences are a significant cause of patient reluctance or procrastination. The incidence of high-grade ovarian cancer (HGOC) in BRCA1 and BRCA2 carriers before age 50 is relatively low (17% and 1.2%, respectively (16)), yet as long as no risk stratification assay exists, the recommendation of RRBSO cannot be individualized. Moreover, as the life-time risk for OVCA is ~40-60% for BRCA1 mutation carriers and ~20-50% for BRCA2 mutation carriers, in most cases it is unnecessary to RRBSO at young age. The dramatic benefit in cancer-free survival from RRBSO needs to be delicately balanced with the reproductive plans and the morbidity of premature menopause, thus presenting both clinicians and patients with a complicated set of considerations. Ideally, high-risk patients will be repeatedly screened using high sensitivity and specificity assay, to enable safe individualization of the timing of RRBSO. To increase the likelihood of diagnosis of early-stage lesions, which are not reflected in the systemic circulation, biomarkers may be detected in liquid biopsy (LB) sample from the gynecological tract cavity. The most common histological subtype of OVCA encountered among high-risk women, invasive serous papillary carcinoma, arises from precursor lesions that develop in the fallopian tube (FT) fimbriae rather than in the ovarian epithelium (6-8). It is, therefore, conceivable that detection of early or premalignant lesions is possible by approaching and sampling the cells of the fimbriae through a simple procedure called utero-tubal lavage (UtL).

Over the past two decades the origin of most HGOC has been proven to be the epithelium of the distal fallopian tube (8). Serous tubal intraepithelial carcinomas (STICs) are the precancerous lesions which tend to shed tumor cells into the tubal lumen and into the pelvis (Morrison J C, et al. Am J Surg Pathol. 2015 Mar. 30; 39(4):442-53; Bijron J, et al. Am J Surg Pathol. 2013 August 37(8):1123-30; Patrono M G, et al. Gynecol Oncol. 2015 Dec. 1; 139(3):568-72) and eventually become invasive and spread to the peritoneum and the regional lymph nodes (Soong T R, et al. Gynecol Oncol. 2019 February; 152(2):426-33.). Mutational evolutionary analyses identify a window of 6 years between development of TP53 mutated precursor and initiation of HGOC (Labidi-Galy S I, et al. Nat Commun. 2017 Dec. 23. 8(1):1093). This knowledge holds an opportunity to utilize proximal liquid biopsies to retrieve tubal cells and their secreted products as a source for early-stage biomarkers. This may potentially lead to the detection at a curable stage and reduce HGOC-specific mortality. Transcriptomic analyses of normal BRCA-mutant compared to BRCA-WT fallopian tube epithelium showed significant differences and differential response to stimuli. Furthermore, the transcriptional program of pre-menopausal BRCA-mutant fallopian tube epithelium shares high similarity with HGOC (Tone A A, et al. Clin Cancer Res. 2008 Jul. 1; 14(13):4067-78. These data raise the hypothesis that BRCA carriers require a specific diagnostic biomarker, which is different than that of an average-risk population. Three proof-of-concept studies showed that TP53-mutated OVCA DNA can be detected from Papanicolaou cytology samples and from vaginal secretions and uterine washings (9-11). However, these studies were performed in advanced stage OVCA patients, with less than 60% sensitivity. Despite the growing interest in tumor circulating DNA as diagnostic biomarkers, the sensitivity of these tests is limited by the requirement of either ultra-deep, error free sequencing, previous recognition of deleterious mutations, and the fact that deep enough sequencing reveals mutations in healthy subjects as well (12).

The present inventors recently disclosed the results of a deep proteomic analysis of the microvesicles fraction of UtL LB collected in Israel, using a modified UtL technique (13). This approach allows detection of thousands of rare tissue-secreted proteins using mass spectrometry-based proteomics, while overcoming the challenge of high-abundance proteins. A 9-protein classifier was further developed by the inventors, using a training set of 24 samples of BRCA-wild type patients and controls (14, 15). An independent set of 268 UtL samples was used for validation and demonstrated 63% sensitivity and 73% specificity.

Currently, there is no screening modality, and given the very poor outcome of OVCA patients, the recommendation for women with germline BRCA mutation and other high-risk populations, is to undergo prophylactic risk-reducing surgical removal of the ovaries and fallopian tubes at age <40. Surgical early menopause adversely affects not only the reproductive plans of some women, but also has detrimental consequences on their general health and quality of life. As a result, some women defer the decision of surgical prophylaxis too long, while others undergo surgery too early, when the risk is marginal. Therefore, there is an unmet need for efficacious screening of high-risk populations, specifically, for individuals determined as a carrier for germline BRCA mutation. These unmet needs are addressed by the present disclosure.

### SUMMARY OF THE INVENTION

A first aspect of the present disclosure relates to a diagnostic method for detecting ovarian cancer in a subject. More specifically, in some embodiments, the method comprising the following steps. A first step (a), involves determining the expression level of at least two biomarker proteins in at least one biological sample of the diagnosed subject, to obtain an expression value for each of these at least two biomarker protein/s. More specifically, the at least two biomarker proteins may comprise, or in some embodiments, may be selected from at least one of the following options. In a first option (i) or model, the biomarker proteins may be at least one of C4b-binding protein beta chain (C4BPB) and Kinesin-like protein Family Member 20B (KIF20B). In the second option (ii) or model, the biomarker proteins may be at least one of, Vacuolar protein sorting-associated protein 11 homolog (VPS11), Cartilage acidic protein 1 (CRTAC1) and Meckelin (TMEM67). In yet the third option (iii) or model, the biomarker proteins may be at least one of Gap junction alpha-1 protein (GJA1) and Plasma membrane calcium-transporting ATPase 4 (ATP2B4). It should be further noted that the option (iv), for any combination of at least two biomarker proteins of any one of the three models (i), (ii) and (iii), may be also used.

In some optional embodiments, the disclosed at least two biomarkers may be specifically applicable for a subject determined as a carrier for at least one mutation in at least one gene associated with high risk for ovarian cancer.

The next step of the diagnostic methods disclosed herein (b), involves determining if the expression value obtained in step (a) for each of the at least two biomarker protein/s is altered (specifically, positive or negative) with respect to a predetermined standard expression value or to an expression value of the biomarker protein/s in at least one control sample.

Thus, an altered expression value of at least two biomarker proteins of at least one of the (i) C4BPB and KIF20B; (ii) VPS11, CRTAC1 and TMEM67; (iii) GJA1 and ATP2B4; and (iv) any combination of at least two biomarker proteins of any one of (i), (ii) and (iii), biomarker protein/s in the sample as compared with a predetermined standard expression value or to an expression value of the biomarker protein/s in at least one control sample, indicates that the diagnosed subject is suffering from ovarian cancer. More specifically, a positive expression value of at least one of (i) C4BPB; (ii) VPS11, CRTAC1 and TMEM67; (iii) GJA1 and ATP2B4 and/or a negative expression value of KIF20B with respect to a predetermined standard expression value or to an expression value of the biomarker protein/s in at least one control sample, indicates that the subject has ovarian cancer. As indicated above, in some embodiments, the diagnosed subject is a carrier of at least one mutation in at least one gene associated with high risk for ovarian cancer, A further aspect of the present disclosure relates to a diagnostic composition comprising at least two detecting molecules or any combination or mixture of plurality of detecting molecules specific for determining the level of expression of at least two biomarker protein/s. In some embodiments, at least two biomarker protein/s may comprise, or in some embodiments, are selected from at least one of: (i) at least one C4BPB and KIF20B; (ii) at least one VPS11, CRTAC1 and TMEM67; (iii) at least one GJA1 and ATP2B4; and (iv) any combination of at least two biomarker proteins of any one of (i), (ii) and (iii) protein/s; In some embodiments, each of the detecting molecules is specific for one of the biomarker protein/s.

Another aspect of the present disclosure relates to a kit comprising: (a), at least two detecting molecules specific for determining the level of expression of at least two biomarker protein/s in a biological sample. In some embodiments the at least two biomarker protein/s may comprise, or in some embodiments, are selected from at least one of: (i) at least one C4BPB and KIF20B; (ii) at least one VPS11, CRTAC1 and TMEM67; (iii) at least one GJA1 and ATP2B4; and (iv) any combination of at least two biomarker proteins of any one of (i), (ii) and (iii) protein/s or any combination thereof; It should be noted that each of the detecting molecule/s is specific for one of the biomarker proteins. In some embodiments, the kits disclosed herein may optionally further comprise at least one of: (b), pre-determined calibration curve/s or predetermined standard/s providing standard expression values of the at least two biomarker/s; and/or (c), at least one control sample.

These and other aspects of the invention will become apparent by the hand of the following description.

### BRIEF DESCRIPTION OF THE DRAWINGS

In order to better understand the subject matter that is disclosed herein and to exemplify how it may be carried out in practice, embodiments will now be described, by way of non-limiting example only, with reference to the accompanying drawings, in which:

FIG. 1A-1D. Proteomic profiling of the microvesicle fraction of UtL liquid biopsy

FIG. 1A. Illustration of the study cohorts, including BRCA-mutant training and validation sets and BRCA WT/unknown validation sets.

FIG. 1B. Principal Component Analysis (PCA) of all normal samples, separated according to BRCA mutation status: mutBRCA1, mutBRCA2, mutBRCA unspecified, and BRCA WT.

FIG. 1C. PCA of all normal samples, separated based on age <50 vs. ≥50.

FIG. 1D. PCA of BRCA mutant samples only, separated based on age <50 vs. >50.

FIG. 2A-2D. Proteomic biomarkers for diagnosis the HGOC in BRCA-mutant population

FIG. 2A. Volcano plot shows the differentially expressed proteins between the patient and control samples, with FDR 0.05 and s0=0.5. Statistically significant proteins are highlighted in dark gray.

FIG. 2B. ROC curve of Model 1 for BRCA carriers

The curve indicates AUC=0.9789 (95% Wald CI: 0.9533, 1.000).

FIG. 2C. ROC curve of Model 2 for BRCA carriers

The curve indicates AUC=0.9683 (95% Wald CI: 0.9379, 0.9988).

FIG. 2D. ROC curve of Model 3 for BRCA carriers

The curve indicates AUC=0.9432 (95% Wald CI: 0.8890, 0.9974).

FIG. 3. ROC curve of Model 4 for BRCA non-carriers

The curve indicates AUC=0.9313 (95% Wald CI: 0.8954, 0.9672).

FIG. 4A-4B. Biomarker protein expression in carriers and non-carriers of BRCA1/2 mutations

FIG. 4A. shows the expression of C4BPB (=protein1061), KIF20B (=Protein2407), VPS11 (=Protein28), CRTAC1 (=Protein71), TMEM67 (=Protein72), GJA1 (=Protein1008) and ATP2B4 (=Protein1108) in UtL samples of BRCA carrier ovarian cancer patients.

FIG. 4B. shows the expression of CTSD (=Protein123), SPTBN2 (=Protein410), ARFIP1 (=Protein1487), MIA3 (=Protein2042), CD109 (=Protein2110), IRGQ (=Protein2291) and GOLPH3 (=Protein2559) in UtL samples of BRCA non-carrier ovarian cancer patients.

FIG. 5A-5D. Validation of the 7-protein classifier for BRCA-mutant population FIG. 5A. Differential expression of each of the 7 proteins in HGOC patients vs. controls (BRCA-mutated). Horizontal line indicates mean value.

FIG. 5B-5D. ROC curves of 3 discriminant models: Model 1, AUC=0.9466 (95% Wald CI: 0.8889, 1.000) (FIG. 5B); Model 2, AUC=1.0 (FIG. 5C); Model 3, AUC=0.9776 (95% Wald CI: 0.9391, 1.000) (FIG. 5D).

FIG. 6. Lack of linkage between alterations in expression (amplification or deletion) of the 7 classifier proteins and BRCA1/2 mutant status TCGA samples with BRCA1/2 mutations are included in the analysis.

FIG. 7. Representative immunohistochemistry stains of morphologically-normal FT fimbria epithelium from BRCA-WT and BRCA-mutant women and HGOC tumor section

Samples were stained with anti-VPS11, anti-ATP2B4, anti-TMEM67 and anti-CRTAC1 antibodies. Cytoplasmic stains for VPS11 and ATP2B4 are consistently lower in the epithelium of fimbriae of BRCA carriers, compared to BRCA-WT epithelium, and also compared to HGOC tumor cells. TMEM67 staining is limited to ciliated epithelial cells in BRCA-WT FT tissues and is expressed in both ciliated and secretory cells in BRCA-mutant compared to BRCA-WT tissues, and diffusely in HGOC tumor cells. CRTAC1 is hardly expressed in BRCA-WT FT fimbriae, focally positive in secretory cells of BRCA-mutant FT fimbriae and in HGOC tumors.

FIG. 8A-8E. Correlation plots of 5 representative UtL samples Sample were prepared and analyzed in two different batches, on different platforms and different technical teams.

FIG. 8A. the plot presents sample 6629;

FIG. 8B. the plot presents sample 4406;

FIG. 8C. the plot presents sample 3238;

FIG. 8D. the plot presents sample 7612;

FIG. 8E. the plot presents sample 3034.

FIG. 9A-9F. Performance of the 7-protein classifier in BRCA-WT or BRCA-unknown population

FIG. 9A. Differential expression of each of the 7 proteins in HGOC patients vs. controls. Horizontal line indicates mean value.

FIG. 9B-9F. ROC curves of 3 discriminant models: Model 1—set 1 and 2 (FIG. 9B and FIG. 9C, respectively); Model 2—set 1 (FIG. 9D); Model 3—set 1 and 2 (FIG. 9E and FIG. 9F, respectively). 95% Wald confidence intervals are indicated by each AUC.

### DETAILED DESCRIPTION OF EMBODIMENTS

The window-of-opportunity for early cancer detection is several years long, during which the tumor is not clinically evident, cannot be detected by imaging and does not have a systemic impact. Given the very poor outcome of ovarian cancer (OVCA) patients, the recommendation for women with germline BRCA mutation and other high-risk populations, is to undergo prophylactic risk-reducing surgical removal of the ovaries and fallopian tubes at age <40. Surgical early menopause adversely affects not only the reproductive plans of some women, but also has deleterious consequences on their general health and quality of life. As a result, some women defer the decision of surgical prophylaxis too long, while others undergo surgery too early, when the risk is marginal. Efforts to develop clinically meaningful biomarkers for early detection of high-grade ovarian cancer (HGOC) have been futile for several decades and there are still no screening guidelines that reduce disease-specific mortality. Transvaginal ultrasound and serum CA125 testing are the standard practice, nonetheless it fails to detect early-stage disease. As knowledge regarding risk modifiers is lacking, personal risk of BRCA-mutation carriers cannot be accurately calculated.

Blood is clearly the most appealing resource for screening of vast populations since it can be easily obtained and handled, however, it is still debatable whether blood-based assays can truly detect HGOC early enough, before metastasis development, to increase cure rates. Proximal liquid biopsies are a fertile ground for discovery of biomarkers.

The inventors propose a minimally invasive technique to sample a liquid biopsy directly from the uterine cavity, which is continuous with the lumen of the site-of-origin of HGOC—the fallopian tube fimbria. This body fluid is more likely to disclose changes in the mullerian tract epithelium, such as early-stage malignancy, before they become evident in the systemic circulation. The proteomic approach captures molecular processes beyond the level of the tumor DNA, such as point mutations, copy number alterations and methylation markers, and may potentially disclose the perturbations in the tumor microenvironment as well. The inventor's protein biomarker can discriminate between patients and controls at high risk due to germline BRCA mutation, with high enough sensitivity and specificity to be considered clinically applicable. A decision support tool to assist BRCA carriers and their physicians determine the individual risk for HGOC and the correct timing for safe RRSO should have a maximal sensitivity and maximal NPV, while specificity may be somewhat compromised, as long as RRSO remains the default solution for women with alarming positive or false-positive results. The inventors analyzed longitudinal samples of healthy pre-menopausal BRCA carriers, and discovered variability in protein intensity that did not affect the prediction but may be related to physiological changes. The inventors currently record the phase of the menstrual cycle and are able to define the best timing for sampling this liquid biopsy in pre-menopausal women. All the participants in this trial follow the recommendation for RRSO before age 40-45, so the chances of detecting latent HGOC while on the trial is extremely low. Moreover, the paucity of stage 1 and STIC lesions is an unsurpassable caveat that has plagued all early detection trials and challenges the ability to prove that the inventor's liquid biopsy technique and classifier genuinely detect clinically latent HGOC. Nonetheless, this innate problem is also the source of motivation for this type of research. This proteomic signature is composed of three individual models that may be integrated to enhance the confidence and can compensate for failure to detect one or more of the proteins, as seen in the present validation data. In future clinical trials the inventors suggest that all cases in which at least one scores is suggestive of HGOC risk, the participant should be referred for subsequent testing (i.e. pelvic MRI). Presumably more data can help define how to best adjust the weight of the three models.

The proteins that compose the signature were selected in an unbiased manner, using logistic regression methods. Their involvement in the carcinogenic process of HGOC has not been investigated yet, but previous studies associated most of them with other types of malignancies. KIF20B is a slow molecular motor protein, involved in cytokinesis, and cerebral cortex development (Abaza A, et al. J Biol Chem. 2003 Jul. 25. 278(30):27844-52). It increases the proliferation of pancreatic and colon and bladder adenocarcinoma, and has been shown to be a poor prognostic marker (Chen J, et al. J Oncol. 2021; Lin W F, et al. Mol Carcinog. 2018 Jul. 1; 57(7):911-25; Kanehira M, et al. Cancer Res. 2007 Apr. 1. 67(7):3276-85=). VPS11 is a vesicle mediated protein trafficking factor, required for fusion of endosomes and autophagosomes with lysosomes (Wartosch L, et al. Traffic. 2015 Jul. 1. 16(7):727-42). It has been shown to facilitate VEGFA secretion, in interaction with FOXM1 (Zhang W, Z et al. Mol Oncol. 2021 May 1. 15(5):1466-85), and regulate several signalling factors and pathways, including Wnt, estrogen receptor α, and NFκB (Segala G, et al. Nat Commun. 2019 Dec. 1. 10(1).). CRTAC1 is a glycosylated extracellular matrix protein expressed in chondrogenic tissue (Steck E, et al. Matrix Biol. 2007 Jan. 26(1):30-41). It has been shown to be down-regulated in bladder cancer, losing its tumor suppressive phenotype (Yang J, et al. Bioengineered. 2021 Dec. 3]; 12(2):9377-89). TMEM67 is required for ciliary structure, length, quantity and function (Picariello T, et al. Cilia. 2014 Jan. 31; 3(1)=). It is also involved in cerebellar development. TMEM67 has been shown to be down-regulated in bladder carcinoma, resulting in poor prognosis (Du E, et al. Urol Oncol. 2017 Apr. 1; 35(4):152.e7-152.e12). GJA1 is a key component in the gap junction complex. GJA1 has been shown to regulate cell proliferation and its down-regulation is correlated with poor prognosis in several types of human cancer, particularly in HGOC (Qiu X, et al. J Cell Physiol. 2016 Jan. 1; 231(1):111-9; Qiu X, et al. Cell Signal. 2015 Oct. 1; 27(10):1956-62). Two additional proteins, C4BPB (which controls the classical pathway of complement activation) and ATP2B4 (play a role in intracellular calcium homeostasis in erythrocytes), have not been implicated in cancer biology. Interestingly, some of these proteins show a different expression trend than previously published, which suggests that the context of the tissue type may be critical to the protein function.

The benefits of the inventor's simplified UtL technique make it feasible in real-world setting: It uses a non-proprietary insemination or pipelle catheter, does not require anesthesia, special equipment, or training. It can be performed by relatively unskilled gynecologist in an office setting, even in nulliparous women. Different independent ethics committees approved the UtL technique for the inventor's trial, as well as for other trials in worldwide (ClinicalTrials.gov: NCT02062697, NCT02039388, NCT02518256, NCT02387645, NCT03606486, NCT04794322, NCT04823871), based on evidence for lack of adverse prognosis of women who undergo hysteroscopy for endometrial carcinoma, despite consequent positive peritoneal cytology (Ribeiro C M, et al. J Minim Invasive Gynecol. 2021 Aug. 1; 28(8):1536-43; Larish A, et al. Int J Gynecol Cancer. 2020 Oct. 1; 30(10):1513-9; Dong H, et al. J Obstet Gynaecol Res. 2021 Sep. 1; 47(9):2969-77.). Extrapolating from the results of the present study regarding safety and stress levels and low complication rates, it is likely to be acceptable for BRCA1/2 germline mutation carriers, as a semi-annual procedure, in cases where delay of RRSO is desired. The proteomic assay may be combined with other methodologies in order to further enhance the diagnostic accuracy. The present disclosure provides a set of ovarian cancer diagnostic signatures based on proteomic profiling of microparticle fraction of liquid biopsy obtained through UtL. Implementation of the diagnostic methods and kits disclosed by the present disclosure clearly enables efficacious screening of high-risk populations, replacing the current practice using CA125 testing and transvaginal ultrasound every 4-6 months, with ~60% sensitivity and specificity and no cancer-specific survival benefit.

To summarize, the novelty of the present disclosure stems from the following aspects: (1) Separate diagnostic models based on mutation status of at least one gene associated with increased or high risk of ovarian cancer, for example, BRCA mutation status, with particularly high sensitivity in the BRCA-mutant population. (2) Utilizing a unique type of LB that is obtained from within the uterine cavity using minimally invasive disease, thus tremendously increasing the chance of detecting biomarkers indicating early-stage disease. (3) Proteomic biomarkers from the microparticle fraction of LB, increasing the sensitivity of detection of tissue-specific factors. The present disclosure provides a diagnostic method for high grade OVCA using body fluids.

A first aspect of the present disclosure relates to a diagnostic method for detecting ovarian cancer in a subject. More specifically, in some embodiments, the method comprising the following steps:

A first step (a), involves determining the expression level of at least two biomarker proteins in at least one biological sample of the diagnosed subject, to obtain an expression value for each of these at least two biomarker protein/s. More specifically, the at least two biomarker proteins may comprise, or in some embodiments, may be selected from at least one of the following options. In a first option (i) or model, the biomarker proteins may be at lest one of C4b-binding protein beta chain (C4BPB) and Kinesin-like protein Family Member 20B (KIF20B). In the second option (ii) or model, the biomarker proteins may be at least one of, Vacuolar protein sorting-associated protein 11 homolog (VPS11), Cartilage acidic protein 1 (CRTAC1) and Meckelin (TMEM67). In yet the third option (iii) or model, the biomarker proteins may be at least one of Gap junction alpha-1 protein (GJA1) and Plasma membrane calcium-transporting ATPase 4 (ATP2B4). It should be further noted that the option (iv), for any combination of at least two biomarker proteins of any one of the three models (i), (ii) and (iii), may be also used. In some optional embodiments, the disclosed signature, specifically or models 1, 2 3 and any combinations thereof as discussed herein, is specifically applicable for a subject determined as a carrier for at least one mutation in at least one gene associated with high risk for cancer, specifically, ovarian cancer. Specifically, carriers of a mutation in hereditary ovarian cancer genes also indicated herein as genetically predisposed population.

In yet some optional either alternative or additional embodiments, the disclosed methods offer an additional model 4. In such specific and non-limiting embodiments, the at least two biomarker proteins may be selected from Cathepsin D (CTSD), Spectrin beta chain, non-erythrocytic 2 (SPTBN2), Arfaptin-1 (ARFIP1), Transport and Golgi organization protein 1 homolog (MIA3), CD109 antigen (CD109), Immunity-related GTPase family Q protein (IRGQ) and Golgi phosphoprotein 3 (GOLPH3) biomarker proteins. In yet some further embodiments, this option may be applicable for a subject determined as not being a carrier for at least one mutation in at least one gene associated with high risk for ovarian cancer.

As indicated above, the disclosed signature of at least two of seven biomarkers may be applicable for carrier subjects. It should be thus appreciated that in some embodiments of the present disclosure the diagnosed subjects are first evaluated or classified as subjects that carry at least one mutation in at least one gene that is associated with increased risk, specifically, for cancer, more specifically, for ovarian cancer.

The next step of the diagnostic methods disclosed herein (b), involves determining if the expression value obtained in step (a) for each of the at least two biomarker protein/s is altered (for example, is positive or negative) with respect to a predetermined standard expression value or to an expression value of the biomarker protein/s in at least one control sample.

It should be noted that an altered expression value of at least two biomarker proteins of at least one of the (i) at least one of C4BPB and KIF20B; (ii) at least one of VPS11, CRTAC1 and TMEM67; (iii) at least one of GJA1 and ATP2B4; and (iv) any combination of at least two biomarker proteins of any one of (i), (ii) and (iii), biomarker protein/s in the sample as compared with a predetermined standard expression value or to an expression value of the biomarker protein/s in at least one control sample, indicates that the diagnosed subject is suffering from ovarian cancer. More specifically, a positive expression value, or in other words, elevated expression, high expression or upregulation of at least one of (i) C4BPB; (ii) at least one of VPS11, CRTAC1 and TMEM67; (iii) at least one of GJA1 and ATP2B4 and/or a negative expression value, or down regulation of KIF20B in the sample as compared with a predetermined standard expression value or to an expression value of the biomarker protein/s in at least one control sample, indicates that the subject has ovarian cancer, thereby diagnosing the subject. As indicated above, the diagnosed subject is determined (e.g., prior to performance of the diagnostic method) as a carrier of at least one mutation in at least one gene associated with high risk for ovarian cancer.

In some optional alternative and/or additional embodiments, where an additional model 4 is used, an altered expression value of at least two of CTSD, SPTBN2, ARFIP1, MIA3, CD109, IRGQ and GOLPH3 biomarker protein/s in said sample, indicates that the diagnosed subject is suffering from ovarian cancer. More specifically, a positive expression value, or in other words, elevated expression, high expression or upregulation of at least one of CTSD, SPTBN2, ARFIP1, MIA3, CD109 and IRGQ, and/or a negative expression value, or down regulation of GOLPH3 as compared to a control, indicates that the subject has ovarian cancer. This alternative model is specifically applicable for and for a subject determined as a non-carrier of at least one mutation in at least one gene associated with high risk for ovarian cancer.

It should be understood that determination of a “positive” or alternatively “negative” expression value with respect to a standard value or a control value may involve in some embodiments comparison of the expression value of the examined sample as obtained in step (a), with the expression value obtained for a control sample, or from any established or predetermined expression value (e.g., a standard value) obtained from a known control (either healthy controls or of subjects suffering from ovarian cancer). Thus, in some embodiments, “positive” is meant an expression value that is higher, increased, elevated, overexpressed in about 5% to 100% or more, specifically, 5%, 10%, 15%, 20%, 25%, 30%, 35%, 40%, 45%, 50%, 55%, 60%, 65%, 70%, 75%, 80%, 85%, 90%, 95%, 100%, when compared to the expression value of a healthy control, any other suitable control or any other predetermined standard. Still further, a “negative” expression value in some embodiments may be a reduced, low, non-existing or lack of expression of a biomarker in about 5% to 100% or more, specifically, 5%, 10%, 15%, 20%, 25%, 30%, 35%, 40%, 45%, 50%, 55%, 60%, 65%, 70%, 75%, 80%, 85%, 90%, 95%, 100%, when compared to the expression value of a healthy control, any other suitable control or any other predetermined standard. A “positive” expression value should be in the range of the expression value of a control patient diagnosed with ovarian cancer, or any other cut off value obtained for a population of ovarian cancer patients. Still further, when the expression value obtained in the examined sample for at least two of C4BPB, VPS11, CRTAC1, TMEM67, GJA1 and ATP2B4 is determined as “positive”, specifically, higher, overexpressed, elevated when compared to a healthy control, the subject is classified as a subject that carry or has an ovarian cancer. It should be noted that in case of biomarkers that display reduced, low or non-existing expression in ovarian cancer, for example, KIF20B, a “negative” expression value should be in the range of the expression value of a control patient diagnosed with ovarian cancer, or any other cut off value obtained for a population of ovarian cancer patients. Still further, in the same manner, where the expression value of at least two of C4BPB, VPS11, CRTAC1, TMEM67, GJA1 and ATP2B4 is determined as “negative”, it should be understood that the expression value is in the range of the expression value of a control healthy subjects or subjects that do not suffer of ovarian cancer, or any other cut off value obtained for a population of healthy subject. Similarly, a “positive” expression value determined in a sample for KIF20B, indicates that the expression value is in the range of a control healthy subjects or subjects that do not suffer of ovarian cancer, or any other cut off value obtained for a population of healthy subject.

Still further, in yet some alternative or additional embodiments where the additional model 4 is used, in case the expression value obtained in the examined sample for at least two of CTSD, SPTBN2, ARFIP1, MIA3, CD109 and IRGQ is determined as “positive”, specifically, higher, overexpressed, elevated when compared to a healthy control, the subject is classified as a subject that carry or has an ovarian cancer. It should be noted that in case of biomarkers that display reduced, low or non-existing expression in ovarian cancer, for example, GOLPH3, a “negative” expression value should be in the range of the expression value of a control patient diagnosed with ovarian cancer, or any other cut off value obtained for a population of ovarian cancer patients. Still further, in the same manner, where the expression value of at least two of CTSD, SPTBN2, ARFIP1, MIA3, CD109, IRGQ is determined as “negative”, it should be understood that the expression value is in the range of the expression value of a control healthy subjects or subjects that do not suffer of ovarian cancer, or any other cut off value obtained for a population of healthy subject. Similarly, a “positive” expression value determined in a sample for GOLPH3, indicates that the expression value is in the range of a control healthy subjects or subjects that do not suffer of ovarian cancer, or any other cut off value obtained for a population of healthy subject.

It should be noted that the detecting molecules may be provided in a diagnostic composition or in a kit either attached to a solid support or alternatively, in a mixture. Thus, the method of the invention encompasses in certain embodiments also the provision of a composition, kit, solid support or mixture comprising at least one detecting molecule specific for at least one of the biomarker proteins of the invention.

More particularly, the method of the invention may use as diagnostic tool, the expression values of each and any one of the marker proteins described herein below or of any combinations thereof.

Thus, in certain embodiments, the methods, compositions and kits of the invention may use as a diagnostic tool the expression value of this biomarker either alone or in any combination with any of the biomarker protein/s disclosed by the invention.

More specifically, C4b-binding protein beta chain (C4BPB) as described herein refers to the human C4BPB (UNITPROT ID: P20851). Still further, the C4BPB is encoded by the amino acid sequence as denoted by Accession number: NM_000716. In more specific embodiments, the C4BPB protein as used herein may comprise the amino acid sequence as denoted by SEQ ID NO. 1, or any homologs, variants or derivatives thereof, and may be encoded by the nucleic acid sequence as denoted by SEQ ID NO. 2, or any homologs, variants or derivatives thereof.

In yet some further embodiments, Kinesin-like protein Family Member 20B (KIF20B) as described herein is the human KIF20B (UNITPROT ID: Q96Q89). Still further, the KIF20B is encoded by the nucleic acid sequence as denoted by Accession number: NM_016195. In more specific embodiments, the KIF20B protein as used herein may comprise the amino acid sequence as denoted by SEQ ID NO. 3, or any homologs, variants or derivatives thereof, and may be encoded by the nucleic acid sequence as denoted by SEQ ID NO. 4, or any homologs, variants or derivatives thereof.

In some embodiments, Vacuolar protein sorting-associated protein 11 homolog (VPS11) as described herein, is the human VPS11 (UNITPROT ID: Q9H270). Still further, in some embodiments the VPS11 is encoded by the nucleic acid sequence as denoted by Accession number: NM_021729. In more specific embodiments, the VPS11 protein as used herein may comprise the amino acid sequence as denoted by SEQ ID NO. 5 or any homologs, variants or derivatives thereof, and may be encoded by the nucleic acid sequence as denoted by SEQ ID NO. 6, or any homologs, variants or derivatives thereof. In certain embodiments, the Cartilage acidic protein 1 (CRTAC1) as described herein, is the human CRTAC1 (UNITPROT ID: Q9NQ79). In yet some further embodiments, the CRTAC1 biomarker is encoded by the nucleic acid sequence as denoted by Accession number: NM_018058. In more specific embodiments, the CRTAC1 protein as used herein may comprise the amino acid sequence as denoted by SEQ ID NO. 7 or any homologs, variants or derivatives thereof, and may be encoded by the nucleic acid sequence as denoted by SEQ ID NO. 8, or any homologs, variants or derivatives thereof. In certain embodiments, the Meckelin (TMEM67) as described herein, is the human TMEM67 (UNITPROT ID: Q5HYA8). In yet some further embodiments, the TMEM67 biomarker is encoded by the nucleic acid sequence as denoted by Accession number: NM_153704

In more specific embodiments, the TMEM67 protein as used herein may comprise the amino acid sequence as denoted by SEQ ID NO. 9, or any homologs, variants or derivatives thereof, and may be encoded by the nucleic acid sequence as denoted by SEQ ID NO. 10, or any homologs, variants or derivatives thereof.

In certain embodiments, the Gap junction alpha-1 protein (GJA1) as described herein is the human GJA1 (UNITPROT ID: Q969M2). In yet some further embodiments, the GJA1 biomarker is encoded by the nucleic acid sequence as denoted by Accession number: NM_032602.

In more specific embodiments, the GJA1 protein as used herein may comprise the amino acid sequence as denoted by SEQ ID NO. 11, or any homologs, variants or derivatives thereof, and may be encoded by the nucleic acid sequence as denoted by SEQ ID NO. 12, or any homologs, variants or derivatives thereof.

In certain embodiments, the Plasma membrane calcium-transporting ATPase 4 (ATP2B4) as described herein, is the human ATP2B4 (UNITPROT ID: P23634). In yet some further embodiments, the ATP2B4 biomarker is encoded by the nucleic acid sequence as denoted by Accession number: NM_001001396. In more specific embodiments, the ATP2B4 protein as used herein may comprise the amino acid sequence as denoted by SEQ ID NO. 13, or any homologs, variants or derivatives thereof, and may be encoded by the nucleic acid sequence as denoted by SEQ ID NO. 14, or any homologs, variants or derivatives thereof.

In some embodiments, the biomarkers indicated above, specifically, at least two of C4BPB, KIF20B, VPS11, CRTAC1, TMEM67, GJA1 and ATP2B4 are particularly applicable in case of samples obtained from subject classified as a carrier of at least one mutation in at least one gene associated with high risk for cancer, specifically, ovarian cancer. The specific associated genes will be discussed in more detail herein after.

In some alternative embodiments, the methods, compositions and kits of the present disclosure may use additional and/or alternative biomarker proteins and specified herein below, specifically, at least two of CTSD, SPTBN2, ARFIP1, MIA3, CD109, IRGQ and GOLPH3. In some embodiments, these biomarker proteins may be particularly applicable in case of samples obtained from subject/s classified as a non-carrier of at least one mutation in at least one gene associated with high risk for cancer, specifically, ovarian cancer.

In certain embodiments, the Cathepsin D (CTSD), as described herein is the human, CTSD (UNITPROT ID: P07339). In yet some further embodiments, the CTSD biomarker is encoded by the nucleic acid sequence as denoted by Accession number: NM_001909.

In more specific embodiments, the CTSD protein as used herein may comprise the amino acid sequence as denoted by SEQ ID NO. 15, or any homologs, variants or derivatives thereof, and may be encoded by the nucleic acid sequence as denoted by SEQ ID NO. 16, or any homologs, variants or derivatives thereof.

In certain embodiments, the Spectrin beta chain, non-erythrocytic 2 (SPTBN2) as described herein is the human SPTBN2 (UNITPROT ID: 015020). In yet some further embodiments, the SPTBN2 biomarker is encoded by the nucleic acid sequence as denoted by Accession number: NM_006946.

In more specific embodiments, the SPTBN2 protein as used herein may comprise the amino acid sequence as denoted by SEQ ID NO. 17, or any homologs, variants or derivatives thereof, and may be encoded by the nucleic acid sequence as denoted by SEQ ID NO. 18, or any homologs, variants or derivatives thereof.

In certain embodiments, the Arfaptin-1 (ARFIP1) as described herein is the human ARFIP1 (UNITPROT ID: P53367). In yet some further embodiments, the ARFIP1 biomarker is encoded by the nucleic acid sequence as denoted by Accession number: NM_014447.

In more specific embodiments, the ARFIP1 protein as used herein may comprise the amino acid sequence as denoted by SEQ ID NO. 19, or any homologs, variants or derivatives thereof, and may be encoded by the nucleic acid sequence as denoted by SEQ ID NO. 20, or any homologs, variants or derivatives thereof.

In certain embodiments, the Transport and Golgi organization protein 1 homolog (MIA3) as described herein is the human MIA3 (UNITPROT ID: Q5JRA6). In yet some further embodiments, the MIA3 biomarker is encoded by the nucleic acid sequence as denoted by Accession number: NM_198551.

In more specific embodiments, the MIA3 protein as used herein may comprise the amino acid sequence as denoted by SEQ ID NO. 21, or any homologs, variants or derivatives thereof, and may be encoded by the nucleic acid sequence as denoted by SEQ ID NO. 22, or any homologs, variants or derivatives thereof.

In certain embodiments, the CD109 antigen (CD109), as described herein is the human CD109 (UNITPROT ID: Q6YHK3). In yet some further embodiments, the CD109 biomarker is encoded by the nucleic acid sequence as denoted by Accession number: NM_133493.

In more specific embodiments, the CD109 protein as used herein may comprise the amino acid sequence as denoted by SEQ ID NO. 23, or any homologs, variants or derivatives thereof, and may be encoded by the nucleic acid sequence as denoted by SEQ ID NO. 24, or any homologs, variants or derivatives thereof.

In certain embodiments, the Immunity-related GTPase family Q protein (IRGQ) as described herein is the human IRGQ (UNITPROT ID: Q8WZA9). In yet some further embodiments, the IRGQ biomarker is encoded by the nucleic acid sequence as denoted by Accession number: NM_001007561.

In more specific embodiments, the IRGQ protein as used herein may comprise the amino acid sequence as denoted by SEQ ID NO. 25, or any homologs, variants or derivatives thereof, and may be encoded by the nucleic acid sequence as denoted by SEQ ID NO. 26, or any homologs, variants or derivatives thereof.

In certain embodiments, the Golgi phosphoprotein 3 (GOLPH3) as described herein is the human GOLPH3 (UNITPROT ID: Q9H4A6). In yet some further embodiments, the GOLPH3 biomarker is encoded by the nucleic acid sequence as denoted by Accession number: NM_022130.

In more specific embodiments, the GOLPH3 protein as used herein may comprise the amino acid sequence as denoted by SEQ ID NO. 27, or any homologs, variants or derivatives thereof, and may be encoded by the nucleic acid sequence as denoted by SEQ ID NO. 28, or any homologs, variants or derivatives thereof.

In certain embodiments, the method as well as the composition and kit of the invention may provide and use detecting molecules specific for at least two, at least three, at least four, at least five, at least six, at least seven biomarkers of the present disclosure and further, detecting molecule/s specific for at least one additional biomarker protein and/or control and/or reference protein. It should be noted that each detecting molecule is specific for one biomarker. In some embodiments, the method as well as the kits of the invention described herein after may provide and use further detecting molecules specific for at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 or more, specifically, 110, 120, 130, 140, 150, 160, 170, 180, 190, 200, 250, 300, 350, 400, 450 and 500 at the most, additional biomarker proteins and/or control and/or reference protein. In some specific and non-limiting embodiments, the methods, compositions and kits of the invention may provide and use in addition to detecting molecules specific for at least one of the biomarkers disclosed herein.

It should be understood that in some embodiments, the methods, compositions and kits of the present disclosure may use and/or comprise at least one additional detecting molecule for at least one additional biomarker and/or reference control protein, provided that the total number of the biomarkers of the invention and the at least one additional biomarker/s and/or reference control protein/s is not more than 500 proteins. Thus, in some embodiments, in case detecting molecules specific for at least two of C4BPB, KIF20B, VPS11, CRTAC1, TMEM67, GJA1 and ATP2B4, the methods, compositions and kits may further contain detecting molecules specific for additional 493, 494, 495, 496, 497 or 498 additional biomarker/s and/or reference control protein/s.

In yet some further embodiments, were also in addition or alternatively, the methods, compositions and kits of the present disclosure use detecting molecules specific for at least two of CTSD, SPTBN2, ARFIP1, MIA3, CD109, IRGQ and GOLPH3, detecting molecules specific for at least one additional biomarker and/or reference control protein may be used, provided that the total number of the biomarkers of the invention and the at least one additional biomarker/s and/or reference control protein/s is not more than 500 proteins. Thus, in some embodiments, in case detecting molecules specific for at least two of CTSD, SPTBN2, ARFIP1, MIA3, CD109, IRGQ and GOLPH3, and optionally, at least two of C4BPB, KIF20B, VPS11, CRTAC1, TMEM67, GJA1 and ATP2B4, may be used, the methods, compositions and kits may further contain detecting molecules specific for additional 486, 487, 488, 489. 490, 491, 492, 493, 494, 495, 496, 497 or 498. additional biomarker/s and/or reference control protein/s.

Thus, in some embodiments, the disclosed methods, compositions and kits are based one determining the expression of at least two, three, four, five, six, or seven of C4BPB, KIF20B, VPS11, CRTAC1, TMEM67, GJA1 and ATP2B4, and at least one additional biomarker and/or reference control protein, provided that the total number of the biomarkers of the invention and the at least one additional biomarker/s and/or reference control protein/s is not more than 500 proteins. Thus, in some embodiments, in case at least two of C4BPB, KIF20B, VPS11, CRTAC1, TMEM67, GJA1 and ATP2B4, are used as biomarker proteins by the methods, compositions and kits, at least one additional biomarker/s and/or reference control protein/s may be further used, and no more than 493, 494, 495, 496, 497 or 498 additional biomarker/s and/or reference control protein/s.

In yet some further embodiments, were also in addition or alternatively, the methods, compositions and kits of the present disclosure are based one determining the expression of at least two, three, four, five, six, or seven of CTSD, SPTBN2, ARFIP1, MIA3, CD109, IRGQ and GOLPH3. at least one additional biomarker and/or reference control protein may be used, provided that the total number of the biomarkers of the invention and the at least one additional biomarker/s and/or reference control protein/s is not more than 500 proteins. Thus, in some embodiments, in case at least two of CTSD, SPTBN2, ARFIP1, MIA3, CD109, IRGQ and GOLPH3, and optionally, at least two of C4BPB, KIF20B, VPS11, CRTAC1, TMEM67, GJA1 and ATP2B4, are used, the methods, compositions and kits may further use (e.g., use s a biomarker, specifically, determine the expression of) 486, 487, 488, 489, 490, 491, 492, 493, 494, 495, 496, 497 or 498, additional biomarker/s and/or reference control protein/s.

In some embodiments, the methods as well as the composition and kits of the invention described herein after, may provide and use detecting molecules specific for at least two of the biomarkers specified herein. In some embodiments, the total number of detecting molecules used in the methods, compositions and kits of the present disclosure may be any number of detecting molecules specific for additional biomarkers that may include further of the biomarkers disclosed by the invention, namely, at least two of C4BPB, KIF20B, VPS11, CRTAC1, TMEM67, GJA1, ATP2B4, and/or alternatively, in case model 4 is used, of CTSD, SPTBN2, ARFIP1, MIA3, CD109, IRGQ and GOLPH3, and any additional biomarker and/or control protein, provided that the total number of different proteins, and/or detecting molecules specific for these proteins (e.g., biomarkers and controls) is no more than 100. In yet some further embodiments, the total number of different proteins, and/or detecting molecules specific for these proteins (e.g., biomarkers and/or controls) is no more than 150. In some further embodiments, the total number of different proteins, or detecting molecules specific for these proteins (e.g., biomarkers and/or controls) is no more than 200. In yet some further embodiments, the total number of different proteins, and/or detecting molecules specific for these proteins (e.g., biomarkers and controls) is no more than 250. In further embodiments, the total number of different proteins, and/or detecting molecules specific for these proteins (e.g., biomarkers and controls) is no more than 300. In yet some further embodiments, the total number of different proteins, and/or detecting molecules specific for these proteins (e.g., biomarkers and controls) is no more than 350. In some embodiments, the total number of different proteins, and/or detecting molecules specific for these proteins (e.g., biomarkers and controls) is no more than 400. In further embodiments, the total number of different proteins, and/or detecting molecules specific for these proteins (e.g., biomarkers and controls) is no more than 450. In yet some further embodiments, the total number of different proteins, or detecting molecules specific for these proteins (e.g., biomarkers and controls) is no more than 500 at the most.

In some embodiments, the methods, as well as the compositions and kits of the invention may provide and use detecting molecules specific for at least one additional biomarker protein and at most, 498 additional marker protein/s. In some specific embodiments, the methods and kit/s of the invention may provide and use detecting molecules specific for at least one of the biomarker proteins of the present disclosure, and detecting molecules specific for at least one additional biomarkers, provided that detecting molecules specific for 100, 150, 200, 250, 300, 350, 384, 400, 450 and 500 at the most biomarker proteins and/or control and/or reference proteins are used.

In yet some further embodiments, it should be understood that the methods of the invention as well as the compositions and kits described herein after, may involve the determination of the expression levels of the biomarker proteins of the invention and/or the use of detecting molecules specific for each of said biomarker proteins. Specifically, at least one, at least two, at least three, at least four, at least five, at least six, at least seven of the biomarker protein/s of the invention that may further comprise any additional biomarker proteins or control reference protein provided that 500 at the most biomarker proteins and/or control reference proteins are used. In yet some further specific and non-limiting embodiments, the method of the invention (as well as any compositions and kits thereof) may use said at least two biomarker proteins of the 2, 3, 4, 5, 6 or 7-signatory biomarkers of the present disclosure.

In some embodiments, the at least one, at least two, at least three, at least four, at least five, at least six, at least seven of the biomarker protein/s of the invention may form at least about 10%, 15%, 20%, 25%, 30%, 35%, 40%, 45%, 50%, 55%, 60%, 65%, 70%, 75%, 80%, 85%, 90%, 95% or 100% of the biomarker proteins determined by the methods of the invention. In yet some further embodiments, the detecting molecules specific for at least one, at least two, at least three, at least four, at least five, at least six, at least seven of the biomarker protein/s of the invention, that are used by the methods of the invention and comprised within any of the compositions and kits of the invention may form at least 10%, 15%, 20%, 25%, 30%, 35%, 40%, 45%, 50%, 55%, 60%, 65%, 70%, 75%, 80%, 85%, 90%, 95% or 100% of detecting molecules used in accordance with the invention. It should be appreciated that for each of the selected biomarker proteins at least one detecting molecule/s may be used. In case more than one detecting molecule is used for a certain biomarker protein, such detecting molecules may be either identical or different.

In yet some further embodiments, at least one detecting molecule may be used for each one of the at least two biomarker proteins of the present disclosure, and optionally, for the at least one additional biomarker protein and/or control reference protein, wherein each of the detecting molecules is specific for only one biomarker or control reference protein. In yet some further embodiments, 1, 2. 3, 4, 5, 6, 7. 8, 9, 10 or more, for example 20, 30, 40, 50, 60, 70, 80, 90, 100 or more different detecting molecules may be used for each of the at least two biomarker proteins of the present disclosure, specifically, at least two of C4BPB, KIF20B, VPS11, CRTAC1, TMEM67, GJA1 and ATP2B4, and for any at least one additional biomarker and/or control (having the limit of up to 500 biomarkers of the present disclosure and additional/biomarkers and/or control or reference proteins. Still further, in some embodiments the disclosed methods compositions and kits are based on at least two of the following biomarkers C4BPB, KIF20B, VPS11, CRTAC1, TMEM67, GJA1 and ATP2B4. In some embodiments, the at least two biomarkers may comprise at least two of C4BPB, KIF20B, VPS11, CRTAC1, TMEM67 and GJA1, with the proviso that ATP2B4 is not included.

In some embodiments, the disclosed methods compositions and kits are based on at least three of the following biomarkers C4BPB, KIF20B, VPS11, CRTAC1, TMEM67, GJA1 and ATP2B4. In some embodiments, the at least three biomarkers may comprise at least three of C4BPB, KIF20B, VPS11, CRTAC1, TMEM67 and GJA1, with the proviso that ATP2B4 is not included.

Still further, in some embodiments, the disclosed methods compositions and kits are based on at least four of the following biomarkers C4BPB, KIF20B, VPS11, CRTAC1, TMEM67, GJA1 and ATP2B4. In some embodiments, the at least four biomarkers may comprise at least four of C4BPB, KIF20B, VPS11, CRTAC1, TMEM67 and GJA1, with the proviso that ATP2B4 is not included.

In some embodiments, the disclosed methods compositions and kits are based on at least five of the following biomarkers C4BPB, KIF20B, VPS11, CRTAC1, TMEM67, GJA1 and ATP2B4. In some embodiments, the at least five biomarkers may comprise at least five of C4BPB, KIF20B, VPS11, CRTAC1, TMEM67 and GJA1, with the proviso that ATP2B4 is not included.

In some embodiments, the disclosed methods compositions and kits are based on at least six of the following biomarkers C4BPB, KIF20B, VPS11, CRTAC1, TMEM67, GJA1 and ATP2B4. In some embodiments, the at least six biomarkers may comprise at least six of C4BPB, KIF20B, VPS11, CRTAC1, TMEM67 and GJA1, with the proviso that ATP2B4 is not included.

Still further, in some embodiments the disclosed methods compositions and kits are based on at least two of the following biomarkers C4BPB, KIF20B, VPS11, CRTAC1, TMEM67, GJA1 and ATP2B4. In some embodiments, the at least two biomarkers may comprise at least two of C4BPB, KIF20B, VPS11, CRTAC1, TMEM67 and ATP2B4, with the proviso that GJA1 is not included.

In some embodiments, the disclosed methods compositions and kits are based on at least three of the following biomarkers C4BPB, KIF20B, VPS11, CRTAC1, TMEM67, GJA1 and ATP2B4. In some embodiments, the at least three biomarkers may comprise at least three of C4BPB, KIF20B, VPS11, CRTAC1, TMEM67 and ATP2B4, with the proviso that GJA1 is not included.

Still further, in some embodiments, the disclosed methods compositions and kits are based on at least four of the following biomarkers C4BPB, KIF20B, VPS11, CRTAC1, TMEM67, GJA1 and ATP2B4. In some embodiments, the at least four biomarkers may comprise at least four of C4BPB, KIF20B, VPS11, CRTAC1, TMEM67 and ATP2B4, with the proviso that GJA1 is not included.

In some embodiments, the disclosed methods compositions and kits are based on at least five of the following biomarkers C4BPB, KIF20B, VPS11, CRTAC1, TMEM67, GJA1 and ATP2B4. In some embodiments, the at least five biomarkers may comprise at least five of C4BPB, KIF20B, VPS11, CRTAC1, TMEM67 and ATP2B4, with the proviso that GJA1 is not included.

In some embodiments, the disclosed methods compositions and kits are based on at least six of the following biomarkers C4BPB, KIF20B, VPS11, CRTAC1, TMEM67, GJA1 and ATP2B4. In some embodiments, the at least six biomarkers may comprise at least six of C4BPB, KIF20B, VPS11, CRTAC1, TMEM67 and ATP2B4, with the proviso that GJA1 is not included.

Still further, in some embodiments the disclosed methods compositions and kits are based on at least two of the following biomarkers C4BPB, KIF20B, VPS11, CRTAC1, TMEM67, GJA1 and ATP2B4. In some embodiments, the at least two biomarkers may comprise at least two of C4BPB, KIF20B, VPS11, CRTAC1, ATP2B4 and GJA1, with the proviso that TMEM67 is not included.

In some embodiments, the disclosed methods compositions and kits are based on at least three of the following biomarkers C4BPB, KIF20B, VPS11, CRTAC1, TMEM67, GJA1 and ATP2B4. In some embodiments, the at least three biomarkers may comprise at least three of C4BPB, KIF20B, VPS11, CRTAC1, ATP2B4 and GJA1, with the proviso that TMEM67 is not included.

Still further, in some embodiments, the disclosed methods compositions and kits are based on at least four of the following biomarkers C4BPB, KIF20B, VPS11, CRTAC1, TMEM67, GJA1 and ATP2B4. In some embodiments, the at least four biomarkers may comprise at least four of C4BPB, KIF20B, VPS11, CRTAC1, ATP2B4 and GJA1, with the proviso that TMEM67 is not included.

In some embodiments, the disclosed methods compositions and kits are based on at least five of the following biomarkers C4BPB, KIF20B, VPS11, CRTAC1, TMEM67, GJA1 and ATP2B4. In some embodiments, the at least five biomarkers may comprise at least five of C4BPB, KIF20B, VPS11, CRTAC1, ATP2B4 and GJA1, with the proviso that TMEM67 is not included.

In some embodiments, the disclosed methods compositions and kits are based on at least six of the following biomarkers C4BPB, KIF20B, VPS11, CRTAC1, TMEM67, GJA1 and ATP2B4. In some embodiments, the at least six biomarkers may comprise at least six of C4BPB, KIF20B, VPS11, CRTAC1, ATP2B4 and GJA1, with the proviso that TMEM67 is not included.

Still further, in some embodiments the disclosed methods compositions and kits are based on at least two of the following biomarkers C4BPB, KIF20B, VPS11, CRTAC1, TMEM67, GJA1 and ATP2B4. In some embodiments, the at least two biomarkers may comprise at least two of C4BPB, KIF20B, VPS11, ATP2B4, TMEM67 and GJA1, with the proviso that CRTAC1 is not included.

In some embodiments, the disclosed methods compositions and kits are based on at least three of the following biomarkers C4BPB, KIF20B, VPS11, CRTAC1, TMEM67, GJA1 and ATP2B4. In some embodiments, the at least three biomarkers may comprise at least three of C4BPB, KIF20B, VPS11, ATP2B4, TMEM67 and GJA1, with the proviso that CRTAC1 is not included.

Still further, in some embodiments, the disclosed methods compositions and kits are based on at least four of the following biomarkers C4BPB, KIF20B, VPS11, CRTAC1, TMEM67, GJA1 and ATP2B4. In some embodiments, the at least four biomarkers may comprise at least four of C4BPB, KIF20B, VPS11, ATP2B4, TMEM67 and GJA1, with the proviso that CRTAC1 is not included.

In some embodiments, the disclosed methods compositions and kits are based on at least five of the following biomarkers C4BPB, KIF20B, VPS11, CRTAC1, TMEM67, GJA1 and ATP2B4. In some embodiments, the at least five biomarkers may comprise at least five of C4BPB, KIF20B, VPS11, ATP2B4, TMEM67 and GJA1, with the proviso that CRTAC1 is not included.

In some embodiments, the disclosed methods compositions and kits are based on at least six of the following biomarkers C4BPB, KIF20B, VPS11, CRTAC1, TMEM67, GJA1 and ATP2B4. In some embodiments, the at least six biomarkers may comprise at least six of C4BPB, KIF20B, VPS11, ATP2B4, TMEM67 and GJA1, with the proviso that CRTAC1 is not included.

Still further, in some embodiments the disclosed methods compositions and kits are based on at least two of the following biomarkers C4BPB, KIF20B, VPS11, CRTAC1, TMEM67, GJA1 and ATP2B4. In some embodiments, the at least two biomarkers may comprise at least two of C4BPB, KIF20B, ATP2B4, CRTAC1, TMEM67 and GJA1, with the proviso that VPS11 is not included.

In some embodiments, the disclosed methods compositions and kits are based on at least three of the following biomarkers C4BPB, KIF20B, VPS11, CRTAC1, TMEM67, GJA1 and ATP2B4. In some embodiments, the at least three biomarkers may comprise at least three of C4BPB, KIF20B, ATP2B4, CRTAC1, TMEM67 and GJA1, with the proviso that VPS11 is not included.

Still further, in some embodiments, the disclosed methods compositions and kits are based on at least four of the following biomarkers C4BPB, KIF20B, VPS11, CRTAC1, TMEM67, GJA1 and ATP2B4. In some embodiments, the at least four biomarkers may comprise at least four of C4BPB, KIF20B, ATP2B4, CRTAC1, TMEM67 and GJA1, with the proviso that VPS11 is not included.

In some embodiments, the disclosed methods compositions and kits are based on at least five of the following biomarkers C4BPB, KIF20B, VPS11, CRTAC1, TMEM67, GJA1 and ATP2B4. In some embodiments, the at least five biomarkers may comprise at least five of C4BPB, KIF20B, ATP2B4, CRTAC1, TMEM67 and GJA1, with the proviso that VPS11 is not included.

In some embodiments, the disclosed methods compositions and kits are based on at least six of the following biomarkers C4BPB, KIF20B, VPS11, CRTAC1, TMEM67, GJA1 and ATP2B4. In some embodiments, the at least six biomarkers may comprise at least six of C4BPB, KIF20B, ATP2B4, CRTAC1, TMEM67 and GJA1, with the proviso that VPS11 is not included.

Still further, in some embodiments the disclosed methods compositions and kits are based on at least two of the following biomarkers C4BPB, KIF20B, VPS11, CRTAC1, TMEM67, GJA1 and ATP2B4. In some embodiments, the at least two biomarkers may comprise at least two of C4BPB, ATP2B4, VPS11, CRTAC1, TMEM67 and GJA1, with the proviso that KIF20B is not included.

In some embodiments, the disclosed methods compositions and kits are based on at least three of the following biomarkers C4BPB, KIF20B, VPS11, CRTAC1, TMEM67, GJA1 and ATP2B4. In some embodiments, the at least three biomarkers may comprise at least three of C4BPB, ATP2B4, VPS11, CRTAC1, TMEM67 and GJA1, with the proviso that KIF20B is not included.

Still further, in some embodiments, the disclosed methods compositions and kits are based on at least four of the following biomarkers C4BPB, KIF20B, VPS11, CRTAC1, TMEM67, GJA1 and ATP2B4. In some embodiments, the at least four biomarkers may comprise at least four of C4BPB, ATP2B4, VPS11, CRTAC1, TMEM67 and GJA1, with the proviso that KIF20B is not included.

In some embodiments, the disclosed methods compositions and kits are based on at least five of the following biomarkers C4BPB, KIF20B, VPS11, CRTAC1, TMEM67, GJA1 and ATP2B4. In some embodiments, the at least five biomarkers may comprise at least five of C4BPB, ATP2B4, VPS11, CRTAC1, TMEM67 and GJA1, with the proviso that KIF20B is not included.

In some embodiments, the disclosed methods compositions and kits are based on at least six of the following biomarkers C4BPB, KIF20B, VPS11, CRTAC1, TMEM67, GJA1 and ATP2B4. In some embodiments, the at least six biomarkers may comprise at least six of C4BPB, ATP2B4, VPS11, CRTAC1, TMEM67 and GJA1, with the proviso that KIF20B is not included.

Still further, in some embodiments the disclosed methods compositions and kits are based on at least two of the following biomarkers C4BPB, KIF20B, VPS11, CRTAC1, TMEM67, GJA1 and ATP2B4. In some embodiments, the at least two biomarkers may comprise at least two of ATP2B4, KIF20B, VPS11, CRTAC1, TMEM67 and GJA1, with the proviso that C4BPB is not included.

In some embodiments, the disclosed methods compositions and kits are based on at least three of the following biomarkers C4BPB, KIF20B, VPS11, CRTAC1, TMEM67, GJA1 and ATP2B4. In some embodiments, the at least three biomarkers may comprise at least three of ATP2B4, KIF20B, VPS11, CRTAC1, TMEM67 and GJA1, with the proviso that C4BPB is not included.

Still further, in some embodiments, the disclosed methods compositions and kits are based on at least four of the following biomarkers C4BPB, KIF20B, VPS11, CRTAC1, TMEM67, GJA1 and ATP2B4. In some embodiments, the at least four biomarkers may comprise at least four of ATP2B4, KIF20B, VPS11, CRTAC1, TMEM67 and GJA1, with the proviso that C4BPB is not included.

In some embodiments, the disclosed methods compositions and kits are based on at least five of the following biomarkers C4BPB, KIF20B, VPS11, CRTAC1, TMEM67, GJA1 and ATP2B4. In some embodiments, the at least five biomarkers may comprise at least five of ATP2B4, KIF20B, VPS11, CRTAC1, TMEM67 and GJA1, with the proviso that C4BPB is not included.

In some embodiments, the disclosed methods compositions and kits are based on at least six of the following biomarkers C4BPB, KIF20B, VPS11, CRTAC1, TMEM67, GJA1 and ATP2B4. In some embodiments, the at least six biomarkers may comprise at least six of ATP2B4, KIF20B, VPS11, CRTAC1, TMEM67 and GJA1, with the proviso that C4BPB is not included.

Still further, the diagnostic methods, diagnostic compositions and diagnostic kits disclosed herein are in some embodiments, provide powerful diagnostic tool for subjects that may be at a high risk or increased risk for cancer, specifically, ovarian cancer. Such subjects may be in some embodiments, subjects that carry at least one mutation in at least one gene associated with increased high risk. Thus, as indicated herein, the subjects are first classified as subjects that carry at least one mutation in at least one gene associated with high risk for ovarian cancer, as compared with average risk for cancer, specifically, ovarian cancer, or as non-carrier subjects.

In yet some further embodiments, an increased risk, higher risk, specifically when compared to subject having an average risk is meant subjects that display increased or elevated risk of more than 50% to have ovarian cancer, specifically, 55, 60, 65, 70, 75, 80, 85, 90, 95 and 99% more risk for ovarian cancer, specifically when compared to subjects classified as having an average risk of 50% or less.

A gene, and more specifically, mutation/s in a specific gene associated with, linked to and/or connected with a disorder, such as cancer, and specifically, ovarian cancer. It is understood that the interchangeably used terms “associated”, “linked” and/or “related”, when referring to at least one mutation in at least one gene herein, mean at least one mutation in at least one gene which at least one of: share causalities, co-exist at a higher than coincidental frequency, with diseases, disorders, conditions, or any pathologies, specifically, cancer, and more specifically, ovarian cancer, or where at least one mutation in at least one gene causes, or leads to, directly, or indirectly to the disease, disorder condition or pathology, specifically, ovarian cancer.

In some embodiments, the gene may be a gene coding for at least one product associated with nucleic acid repair mechanism.

Still further, in sone embodiments, the mutations are in at least one gene encoding a protein involved in DNA repair mechanisms and/or DNA repair pathways, including homologous recombination, non-homologous end joining, single strand annealing and checkpoint regulation.

Still further, in some embodiments, the mutations may be in at least one mismatch repair (MMR) gene. More specifically, the DNA mismatch repair system is i a system for recognizing and repairing erroneous insertion, deletion, and mis-incorporation of bases that can arise during DNA replication and recombination, as well as repairing some forms of DNA damage. This system is a bidirectional excision-resynthesis system that is initiated at a defined strand scission that is 3′- or 5′- of a mismatch and the excision tract extends to a nonspecific point just past the mismatch.

In some embodiments, a gene associated with high risk for ovarian cancer as used herein, may be at least one of Breast cancer type 1 susceptibility protein (BRCA1), Breast cancer type 2 susceptibility protein (BRCA2) BRCA1-associated RING domain protein 1 (BARD1), BRCA1 Interacting Helicase 1 (BRIP1), Checkpoint kinase 2(CHEK2), Double-strand break repair protein MRE11 (MRE11A), mutS homolog 6 (MSH6), nibrin (NBN), Partner and localizer of BRCA2, (PALB2), RAD50 Double Strand Break Repair Protein (RAD50), RAD51 Paralog C (RAD51C), RAD51 Paralog D (RAD51D), Transformation-Related Protein 53 (TP53), Adenomatous polyposis coli (APC), ataxia-telangiectasia mutated (ATM), Dicer 1, Ribonuclease III (DICER1) and mutY DNA glycosylase (MUTYH), and/or MMR genes, specifically, at least one of: mutL homolog 1 (MLH1), MutS homolog 2 (MSH2), MutS homolog 6 (MSH6), and PMS1 Homolog 2, Mismatch Repair System Component (PMS2).

In some embodiments, a gene associated with high risk for ovarian cancer may be at least one of Breast cancer type I susceptibility protein (BRCA1) and Breast cancer type 2 susceptibility protein (BRCA2).

In some embodiments, the subject is classified as a subject that carry at least one mutation in at least one MMR gene. In yet some further embodiments, at least one MMR gene associated with Lynch syndrome. Still further, in some embodiments MMR gene/s applicable in the methods, compositions and kits of the present disclosure may comprise at least one of: MLH1, MSH2, MSH6, and PMS2.

In some alternative and optional embodiments, the methods disclosed herein may further comprise an additional step of classifying the diagnosed subject, or determining if the subject is a carrier of at least one mutation in at least one gene associated with high risk, or specifically, if the subject is a carrier of at least one mutation in at least one of BRCA1 and/or BRCA2 gene/s, BRIP1, PALB2, RAD51, BARD1, CHEK2 and MMR genes such as MLH1, MSH2, MSH6, and PMS2.

In some embodiments, BRCA1 as used herein is referred to the human BRCA1 gene having the cytogenetic location: 17q21.31 and genomic coordinates (GRCh38): 17:43,044,295-43,170,327. In yet some further embodiments, the BRCA1 gene comprise the nucleic acid sequence as denoted by SEQ ID NO: 29. Still further, in some embodiments, BRCA2 as used herein is referred to the human BRCA1 gene having the cytogenetic location: 13q13.1, and Genomic coordinates (GRCh38): 13:32,315,508-32,400,268. In yet some further embodiments, the BRCA1 gene comprise the nucleic acid sequence as denoted by SEQ ID NO: 30.

In some embodiments, MSH2 as used herein is referred to the human MSH2 gene having the cytogenetic location: 2p21-p16.3, and genomic coordinates (GRCh38): 2:47,403,067-47,709,830. In yet some further embodiments, the MSH2 gene comprise the nucleic acid sequence as denoted by SEQ ID NO: 31.

Still further, in some embodiments, the MLH1, as used herein is referred to the human MLH1 gene having the cytogenetic location: 3p22.2, and the genomic coordinates (GRCh38): 3:36,993,466-37,050,846. In yet some further embodiments, the MLH1 gene comprise the nucleic acid sequence as denoted by SEQ ID NO: 32.

In some embodiments, PMS2 as used herein is referred to the human PMS2 gene having the cytogenetic location: 7p22.1 and genomic coordinates (GRCh38): 7:5,970,925-6,009,106. In yet some further embodiments, the PMS2 gene comprise the nucleic acid sequence as denoted by SEQ ID NO: 33.

In some embodiments, MSH6 as used herein is referred to the human MSH6 gene having the cytogenetic location: 2p16.3 and genomic coordinates (GRCh38): 2:47,783,145-47,810,101. In yet some further embodiments, the MSH6 gene comprise the nucleic acid sequence as denoted by SEQ ID NO: 34.

In some embodiments, CHEK2 as used herein is referred to the human CHEK2 gene having the cytogenetic location: 22q12.1 and genomic coordinates (GRCh38): 22:28,687,743-28,741,834. In yet some further embodiments, the CHEK2 gene comprise the nucleic acid sequence as denoted by SEQ ID NO: 35.

In some embodiments, RAD51 as used herein is referred to the human RAD51 gene having the cytogenetic location: 1515.1 and genomic coordinates (GRCh38): 15:40,694,733-40,732,340. In yet some further embodiments, the RAD51 gene comprise the nucleic acid sequence as denoted by SEQ ID NO: 36.

In some embodiments, BRIP1 as used herein is referred to the human BRIP1 gene having the cytogenetic location: 17q23.2 and genomic coordinates (GRCh38): 17:61,679,139-61,863,528. In yet some further embodiments, the BRIP1 gene comprise the nucleic acid sequence as denoted by SEQ ID NO: 37.

In some embodiments, PALB2 as used herein is referred to the human PALB2 gene having the cytogenetic location: 16p12.2 and genomic coordinates (GRCh38): 16:23,603,165-23,641,310. In yet some further embodiments, the PALB2 gene comprise the nucleic acid sequence as denoted by SEQ ID NO: 38.

In some embodiments, BARD1 as used herein is referred to the human BARD1 gene having the cytogenetic location: 2q35 and genomic coordinates (GRCh38): 2:214,725,646-214,809,683. In yet some further embodiments, the BARD1 gene comprise the nucleic acid sequence as denoted by SEQ ID NO: 39.

It should be understood that the step of determining if the subject is a carrier of at least one mutation, specifically at least one mutation associated with cancer, specifically, ovarian cancer, and/or classifying the subject as a carrier or a non-carrier of the specific mutation in any of the specified genes may be performed by any appropriate means. Specifically, any cytogenetic and/or molecular methods may be applicable in the methods disclosed herein, as well as in the compositions and kits, including direct sequencing, DNA hybridization and/or restriction enzyme digestion methods. It should be appreciated however that additional methods such as FISH (Fluorescence in situ hybridization); CGH (Comparative genomic hybridization); RELP (Restriction fragment length polymorphism); ARMS (Amplification refractory mutation system); PCR (Polymerase chain reaction); MLPA (Multiplex ligation-dependent probe amplification); DGGE (Denaturing Gradient Gel Electrophoresis); SSCP (Single Strand Conformational Polymorphism); CCM (Chemical cleavage of mismatch); PTT (Protein truncation test); OLA (Oligonucleotide ligation assay); next-generation sequencing (NGS); whole-genome sequencing (WGS), whole exome sequencing (WES), transcriptome sequencing, methylome, and the like.

It is suggested by the inventors that this 2, 3, 4, 5, 6 or 7-protein signature described above, or any of the subgroup specified herein, specifically, at least two, at least three, at least four, at least five, at least six, at least seven biomarker proteins or more, (but no more than 100, 200, 30, 400, or 500), may enable early detection of ovarian cancer. The inventors envision that this signature may be implemented into clinical applications as established herein, to determine presence of ovarian cancer already at an early stage thereby potentially increasing survival of HGOC (high grad ovarian cancer), or OVCA patients but also limiting the need of risk-reducing bilateral salpingo oophorectomy (RRBSO) in high-risk population.

The term “cancer” is used herein interchangeably with the term “tumor” and denotes a mass of tissue found in or on the body that is made up of abnormal cells. As used herein, the term “ovarian cancer” is used herein interchangeably with the term “fallopian tube cancer” or “primary peritoneal cancer” referring to a cancer that develops from ovary tissue, fallopian tube tissue or from the peritoneal lining tissue.

Early symptoms can include bloating, abdominopelvic pain, and pain in the side. The most typical symptoms of ovarian cancer include bloating, abdominal or pelvic pain or discomfort, back pain, irregular menstruation or postmenopausal vaginal bleeding, pain or bleeding after or during sexual intercourse, difficulty eating, loss of appetite, fatigue, diarrhea, indigestion, heartburn, constipation, nausea, early satiety, and possibly urinary symptoms (including frequent urination and urgent urination); typically these symptoms are caused by a mass pressing on the other abdominopelvic organs or from metastases.

The most common type of ovarian cancer, comprising more than 95% of cases, is epithelial ovarian carcinoma. These tumors are believed to start in the cells covering the ovaries, and a large proportion may form at end of the fallopian tubes. Less common types of ovarian cancer include germ cell tumors and sex cord stromal tumors.

It must be appreciated that the methods, compositions and kits of the invention may be applicable for invasive as well as non-invasive ovarian carcinoma. When referring to “non-invasive” cancer it should be noted as a cancer that do not grow into or invade normal tissues within or beyond the primary location, for example the ovary or the fallopian tube.

When referring to “invasive cancers” it should be noted as cancer that invades and grows in normal, healthy tissues to form metastasis.

As used herein the term “metastatic cancer” or “metastatic status” refers to a cancer that has spread from the place where it first started to another place in the body. Such a tumor formed by metastatic cancer cells is called a metastatic tumor or a metastasis.

Metastasis in ovarian cancer is very common in the abdomen, and occurs via exfoliation, where cancer cells burst through the ovarian capsule and are able to move freely throughout the peritoneal cavity. Ovarian cancer metastases usually grow on the surface of organs rather than the inside; they are also common on the omentum and the peritoneal lining. Cancer cells can also travel through the lymphatic system and metastasize to lymph nodes connected to the ovaries via blood vessels; i.e., the lymph nodes along the infundibulo-pelvic ligament, the broad ligament, and the round ligament. The most commonly affected groups include the paraaortic, hypogastric, external iliac, obturator, and inguinal lymph nodes. In most cases, ovarian cancer does not metastasize to the liver, lung, brain, or kidneys at time of diagnosis; this differentiates ovarian cancer from many other forms of cancer.

Ovarian cancers are classified according to the microscopic appearance of their structures (histology or histopathology). It must be understood that the methods, compositions and kits of the invention may be applicable for the diagnosis of ovarian carcinoma of any of histological subtypes specified herein after. Epithelial ovarian carcinoma, is the most common type of ovarian cancer, representing approximately 90% of ovarian cancers. It includes serous carcinoma, endometrioid carcinoma, clear cell carcinoma, and mucinous cystadenocarcinoma. Less common tumors are malignant Brenner tumor and transitional cell carcinoma of the ovary. Low-grade serous carcinoma is less aggressive than high-grade serous carcinomas, though it does not typically respond well to chemotherapy or hormonal treatments.

About two-thirds of women with epithelial ovarian carcinoma, are diagnosed with serous carcinoma.

Small-cell ovarian carcinoma is rare and aggressive, with two main subtypes: hypercalcemic and pulmonary. It is typically fatal within 2 years of diagnosis.

Hypercalcemic small cell ovarian carcinoma overwhelmingly affects those in their 20s, causes high blood calcium levels, and affects one ovary. Pulmonary small cell ovarian cancer usually affects both ovaries of older women and looks like oat-cell carcinoma of the lung.

Primary peritoneal carcinoma develops from the peritoneum. It can develop even after the ovaries have been removed and may appear similar to mesothelioma.

Clear-cell ovarian carcinomas may be related to endometriosis. Clear-cell adenocarcinomas are histopathologically similar to other clear cell carcinomas, with clear cells and hobnail cells. They represent approximately 5-10% of epithelial ovarian cancers and are associated with endometriosis in the pelvic cavity.

Endometrioid adenocarcinomas make up approximately 15-20% of epithelial ovarian cancers. These tumors frequently co-occur with endometriosis or endometrial cancer.

Mixed mullerian tumors make up less than 1% of ovarian cancer. They have epithelial and mesenchymal cells visible.

Mucinous tumors include mucinous adenocarcinoma and mucinous cystadenocarcinoma.

Mucinous adenocarcinomas make up 5-10% of epithelial ovarian cancers. Histologically, they are similar to intestinal or cervical adenocarcinomas, and are often actually metastases of appendiceal or colon cancers.

Pseudomyxoma peritonei refers to a collection of encapsulated mucous or gelatinous material in the abdominopelvic cavity, which is very rarely caused by a primary mucinous ovarian tumor.

Undifferentiated cancers—those where the cell type cannot be determined—make up about 10% of epithelial ovarian cancers. When examined under the microscope, these tumors have very abnormal cells that are arranged in clumps or sheets.

Malignant Brenner tumors are rare. Histologically, they have dense fibrous stroma with areas of transitional epithelium, and some squamous differentiation. To be classified as a malignant Brenner tumor, it must have Brenner tumor foci and transitional cell carcinoma. The transitional cell carcinoma component is typically poorly differentiated and resembles urinary tract cancer.

Transitional cell carcinomas represent less than 5% of ovarian cancers. Histologically, they appear similar to bladder carcinoma. The prognosis is intermediate—better than most epithelial cancers but worse than malignant Brenner tumors.

Sex cord-stromal tumor, including estrogen-producing granulosa cell tumor, the benign thecoma, and virilizing Sertoli-Leydig cell tumor or arrhenoblastoma, accounts for 7% of ovarian cancers. They occur most frequently in women between 50 and 69 years of age, but can occur in women of any age, including young girls. They are not typically aggressive and are usually unilateral; they are therefore usually treated with surgery alone. Sex cord-stromal tumors are the main hormone-producing ovarian tumors.

Granulosa cell tumors are the most common sex-cord stromal tumors, making up 70% of cases, and are divided into two histologic subtypes: adult granulosa cell tumors, which develop in women over 50, and juvenile granulosa tumors, which develop before puberty or before the age of 30. Both develop in the ovarian follicle from a population of cells that surrounds germinal cells.

Germ cell tumors of the ovary develop from the ovarian germ cells. Germ cell tumor accounts for about 30% of ovarian tumors, but only 5% of ovarian cancers, because most germ-cell tumors are teratomas and most teratomas are benign. Malignant teratomas tend to occur in older women, when one of the germ layers in the tumor develops into a squamous cell carcinoma. Germ-cell tumors tend to occur in young women (20s-30s) and girls, making up 70% of the ovarian cancer seen in that age group. Germ-cell tumors can include dysgerminomas, teratomas, yolk sac tumors/endodermal sinus tumors, and choriocarcinomas, when they arise in the ovary. Some germ-cell tumors have an isochromosome 12, where one arm of chromosome 12 is deleted and replaced with a duplicate of the other.

Dysgerminoma accounts for 35% of ovarian cancer in young women and is the most likely germ cell tumor to metastasize to the lymph nodes; nodal metastases occur in 25-30% of cases. These tumors may have mutations in the KIT gene, a mutation known for its role in gastrointestinal stromal tumor. People with an XY karyotype and ovaries (gonadal dysgenesis) or an X,0 karyotype and ovaries (Turner syndrome) who develop a unilateral dysgerminoma are at risk for a gonadoblastoma in the other ovary, and in this case, both ovaries are usually removed when a unilateral dysgerminoma is discovered to avoid the risk of another malignant tumor. Gonadoblastomas in people with Swyer or Turner syndrome become malignant in approximately 40% of cases. However, in general, dysgerminomas are bilateral 10-20% of the time.

Choriocarcinoma can occur as a primary ovarian tumor developing from a germ cell, though it is usually a gestational disease that metastasizes to the ovary. Primary ovarian choriocarcinoma has a poor prognosis and can occur without a pregnancy. They produce high levels of hCG and can cause early puberty in children or menometrorrhagia (irregular, heavy menstruation) after menarche.

Immature, or solid, teratomas are the most common type of ovarian germ cell tumor, making up 40-50% of cases. Teratomas are characterized by the presence of disorganized tissues arising from all three embryonic germ layers: ectoderm, mesoderm, and endoderm; immature teratomas also have undifferentiated stem cells that make them more malignant than mature teratomas (dermoid cysts). The different tissues are visible on gross pathology and often include bone, cartilage, hair, mucus, or sebum, but these tissues are not visible from the outside, which appears to be a solid mass with lobes and cysts.

Mature teratomas, or dermoid cysts, are rare tumors consisting of mostly benign tissue that develop after menopause. The tumors consist of disorganized tissue with nodules of malignant tissue, which can be of various types. The most common malignancy is squamous cell carcinoma, but adenocarcinoma, basal-cell carcinoma, carcinoid tumor, neuroectodermal tumor, malignant melanoma, sarcoma, sebaceous tumor, and struma ovarii can also be part of the dermoid cyst.

Yolk sac tumors, formerly called endodermal sinus tumors, make up approximately 10-20% of ovarian germ cell malignancies, and have the worst prognosis of all ovarian germ cell tumors. They occur both before menarche (in one-third of cases) and after menarche (the remaining two-thirds of cases). Half of people with yolk sac tumors are diagnosed in stage I. Typically, they are unilateral until metastasis, which occurs within the peritoneal cavity and via the bloodstream to the lungs. Yolk sac tumors grow quickly and recur easily, and are not easily treatable once they have recurred.

Embryonal carcinomas, a rare tumor type usually found in mixed tumors, develop directly from germ cells but are not terminally differentiated; in rare cases they may develop in dysgenetic gonads. They can develop further into a variety of other neoplasms, including choriocarcinoma, yolk sac tumor, and teratoma. They occur in younger people, with an average age at diagnosis of 14, and secrete both alpha-fetoprotein (in 75% of cases) and hCG.

Polyembryomas, the most immature form of teratoma and very rare ovarian tumors, are histologically characterized by having several embryo-like bodies with structures resembling a germ disk, yolk sac, and amniotic sac. Syncytiotrophoblast giant cells also occur in polyembryomas.

Primary ovarian squamous cell carcinomas are rare and have a poor prognosis when advanced. More typically, ovarian squamous cell carcinomas are cervical metastases, areas of differentiation in an endometrioid tumor, or derived from a mature teratoma.

Mixed tumors contain elements of more than one of the above classes of tumor histology. To be classed as a mixed tumor, the minor type must make up more than 10% of the tumor. Though mixed carcinomas can have any combination of cell types, mixed ovarian cancers are typically serous/endometrioid or clear cell/endometrioid. Mixed germ cell tumors make up approximately 25-30% of all germ cell ovarian cancers, with combinations of dysgerminoma, yolk sac tumor, and/or immature teratoma.

Ovarian cancer can also be a secondary cancer, the result of metastasis from a primary cancer elsewhere in the body. About 7% of ovarian cancers are due to metastases, while the rest are primary cancers. Common primary cancers are breast cancer, colon cancer, appendiceal cancer, and stomach cancer (primary gastric cancers that metastasize to the ovary are called Krukenberg tumors). Krukenberg tumors have signet ring cells and mucinous cells. Endometrial cancer and lymphomas can also metastasize to the ovary.

It should be appreciated that the methods, compositions and kits of the invention may be applicable for the diagnosis of primary, as well as secondary ovarian carcinoma as discussed herein. Low malignant potential (LMP) ovarian tumors, also called borderline tumors, have some benign and some malignant features. LMP tumors make up approximately 10%-15% of all ovarian tumors. They develop earlier than epithelial ovarian cancer, around the age of 40-49. They typically do not have extensive invasion; 10% of LMP tumors have areas of stromal microinvasion (<3 mm, <5% of tumor). LMP tumors have other abnormal features, including increased mitosis, changes in cell size or nucleus size, abnormal nuclei, cell stratification, and small projections on cells (papillary projections). Serous and/or mucinous characteristics can be seen on histological examination, and serous histology makes up the overwhelming majority of advanced LMP tumors. More than 80% of LMP tumors are Stage I; 15% are stage II and III and less than 5% are stage IV. Implants of LMP tumors are often non-invasive.

Ovarian cancer is staged using the FIGO staging system or using the AJCC/TNM staging system.

FIGO stages of ovarian cancer are as follows: at stage I, cancer is completely limited to the ovary. At stage IA, it involves one ovary, the capsule is intact, there is no tumor on ovarian surface, washings are negative. At stage IB, cancer involves both ovaries; the capsule is intact, there is no tumor on ovarian surface, washings are negative. At stage IC, tumor involves one or both ovaries. At stage IC1, there is surgical spill. At stage IC2, the capsule has ruptured, or tumor are on ovarian surface. At stage IC3, there are positive ascites or washings. A stage II, one can observe pelvic extension of the tumor (must be confined to the pelvis) or primary peritoneal tumor, it involves one or both ovaries. At stage IIA, tumor is found on uterus or fallopian tubes. At stage IIB, tumor appears elsewhere in the pelvis. At stage III, cancer is found outside the pelvis or in the retroperitoneal lymph nodes, it involves one or both ovaries. At stage IIIA, metastasis appear in retroperitoneal lymph nodes or microscopic extrapelvic metastasis. At stage IIIA1, metastasis is in retroperitoneal lymph nodes. At stage IIIA1(i) the metastasis is less than 10 mm in diameter, at stage IIIA1(ii) the metastasis is greater than 10 mm in diameter. At stage IIIA2, there is microscopic metastasis in the peritoneum, regardless of retroperitoneal lymph node status. At stage IIIB, metastasis appears in the peritoneum less than or equal to 2 cm in diameter, regardless of retroperitoneal lymph node status; or metastasis to liver or spleen capsule. At stage IIIC, metastasis appears in the peritoneum greater than 2 cm in diameter, regardless of retroperitoneal lymph node status; or metastasis to liver or spleen capsule. At stage IV, distant metastasis can be observed (i.e. outside of the peritoneum). At stage IVA, one can observe pleural effusion containing cancer cells. At stage IVB, there is metastasis to distant organs (including the parenchyma of the spleen or liver), or metastasis to the inguinal and extra-abdominal lymph nodes.

The AJCC/TNM staging system indicates where the tumor has developed, spread to lymph nodes, and metastasis AJCC/TNM stages of ovarian cancer are as following: at stage T, primary tumor can be observed. At stage T1, the tumor is limited to ovary/ovaries. At stage T1a, one ovary has intact capsule, no surface tumor, and ascites/peritoneal washings are negative. At stage Tib, both ovaries have intact capsules, no surface tumor, and ascites/peritoneal washings are negative. At stage T1c, one or both ovaries have ruptured capsule or capsules, surface tumor, ascites/peritoneal washings are positive. At stage T2, tumor is in ovaries and pelvis (extension or implantation). At stage T2a, there is expansion to the uterus or the Fallopian tubes, ascites/peritoneal washings are negative. At stage T2b, there is expansion in other pelvic tissues, ascites/peritoneal washings are negative. At stage T2c, there is expansion to any pelvic tissue, ascites/peritoneal washings are positive. At stage T3, the tumor is in ovaries and has metastasized outside the pelvis to the peritoneum (including the liver capsule). At stage T3a, microscopic metastasis is observed. At stage T3b, macroscopic metastasis is less than 2 cm diameter. At stage T3c, macroscopic metastasis is greater than 2 cm diameter. At stage N, regional lymph node metastasis is observed. At stage N1, metastasis is present. At stage M, there is distant metastasis. At stage M0, no metastasis is observed. At stage M1, metastasis is present (excluding liver capsule, including liver parenchyma and cytologically confirmed pleural effusion).

In addition to being staged, like all cancers, ovarian cancer is also graded. The histologic grade of a tumor measures how abnormal or malignant its cells look under the microscope. The four grades indicate the likelihood of the cancer to spread and the higher the grade, the more likely for this to occur. Grade 0 is used to describe noninvasive tumors. Grade 0 cancers are also referred to as borderline tumors. Grade 1 tumors have well differentiated cells (look very similar to the normal tissue) and are the ones with the best prognosis. Grade 2 tumors are also called moderately well-differentiated and they are made up of cells that resemble the normal tissue. Grade 3 tumors have the worst prognosis and their cells are abnormal, referred to as poorly differentiated.

It should be appreciated that the methods, compositions and kits of the invention may be applicable for the diagnosis or ovarian carcinoma, specifically, high grade epithelial ovarian cancer.

In accordance with some embodiments, in the first step (a) of the method of the invention, the expression level of at least one of the biomarker proteins described herein is being determined. The terms “level of expression” or “expression level” are used interchangeably and generally refer to a numerical representation of the amount (quantity) of an amino acid product or polypeptide or protein in a biological sample. In yet some further embodiments, the “level of expression” or “expression level” refers to the numerical representation of the amount (quantity) of polynucleotide which may be gene in a biological sample.

“Expression” generally refers to the process by which gene-encoded information is converted into the structures present and operating in the cell. For example, gene expression values may be measured in the protein level, for example by MS methods or alternatively by immunological methods. Alternatively, the expression may be measured in the nucleic acid level, for example using Real-Time Polymerase Chain Reaction, sometimes also referred to as RT-PCR or quantitative PCR (qPCR). The luminosity in case of RT-PCR, or any other tag is captured by a detector that converts the signal intensity into a numerical representation which is said expression value, in terms of biomarker protein or a gene. Therefore, according to the invention “expression” of a gene, specifically, any gene encoding any of the biomarker proteins of the invention may refer to transcription into a polynucleotide and translation into a polypeptide. Fragments of the transcribed polynucleotide, the translated protein, or the post-translationally modified protein shall also be regarded as expressed whether they originate from a transcript generated by alternative splicing or a degraded transcript, or from a post-translational processing of the protein, e.g., by proteolysis. Methods for determining the level of expression of the biomarkers of the invention will be described in more detail herein after. It should be appreciated that the methods of the invention, as well as the compositions and kits disclosed herein after, refer to the level of the biomarker protein/s in the sample. It should be understood that in certain embodiments, the “expression level” as referred to herein, simply refers to the levels, amount, quantity of the biomarker protein/s in the sample, specifically where the “expression level” is determined at the protein level. The determined level and/or amount reflects not only the level of expression, but may also reflect the stability of the biomarker protein.

The expression level of the biomarker proteins of the invention is determined to obtain an expression value. The term “expression value” refers to the result of a calculation, that uses as an input the “level of expression” or “expression level” obtained experimentally. It should be appreciated that in some optional embodiments, determination of the expression value may further involves normalizing the “level of expression” or “expression level” by at least one normalization step as detailed herein, where the resulting calculated value termed herein “expression value” is obtained.

More specifically, as used herein, “normalized values” in some embodiments, are the quotient of raw expression values of marker proteins, divided by the expression value of a control reference protein from the same sample. Any assayed sample may contain more or less biological material than is intended, due to human error and equipment failures. Importantly, the same error or deviation applies to both the marker protein of the invention and to the control reference protein, whose expression is essentially constant. Thus, division of the marker protein raw expression value by the control reference protein raw expression value yields a quotient which is essentially free from any technical failures or inaccuracies (except for major errors which destroy the sample for testing purposes) and constitutes a normalized expression value of said marker protein. This normalized expression value may then be compared with normalized cutoff values, i.e., cutoff values calculated from normalized expression values. In certain embodiments, the control reference protein may be a protein that maintains stable in all samples analyzed. Still further, in some embodiments, normalization may be performed using a sum/average of multiple proteins.

Normalized biomarker protein expression level values that are higher (positive) or lower (negative) in comparison with a corresponding predetermined standard expression value or a cut-off value in a control sample predict to which population of subjects, either healthy or diseased, the tested sample belongs, and in some embodiments, may even reflect the disease stage, or the metastatic status of the subject.

It should be appreciated that an important step in the method of the inventions is determining whether the expression value of any one of the biomarker proteins is changed or different when compared to a pre-determined cut off or is within the range of expression of such cutoff. Alternatively, or in addition, the expression value may be compared to the expression value of a control sample, for example, a sample obtained from a healthy subject or from a subject that is not affected by ovarian cancer.

Thus, in yet more specific embodiments, the second step (b) of the method of the invention involves comparing the expression values determined for the tested sample with predetermined standard values or cutoff values, or alternatively, with expression values of at least one control sample. As used herein the term “comparing” denotes any examination of the expression level and/or expression values obtained in the samples of the invention as detailed throughout in order to discover similarities or differences between at least two different samples. It should be noted that in some embodiments, comparing according to the present invention encompasses the possibility to use a computer-based approach.

As described hereinabove, the method of the invention refers to a predetermined cutoff value/s. It should be noted that a “cutoff value”, sometimes referred to simply as “cutoff” herein, is a value that meets the requirements for both high diagnostic sensitivity (true positive rate) and high diagnostic specificity (true negative rate).

It should be noted that the terms “sensitivity” and “specificity” are used herein with respect to the ability of one or more markers, to correctly classify a sample as belonging to a pre-established population associated with ovarian cancer, specifically, OVCA (or type II), more specifically, HGOC, or alternatively, to a pre-established population of healthy subjects or subjects that are not affected by OVCA, specifically, HGOC. In other words, to correctly classify a sample as a sample of a subject affected by ovarian cancer or alternatively as a subject that is not affected by ovarian cancer (either healthy or not).

“Sensitivity” indicates the performance of the biomarker of the invention, with respect to correctly classifying samples as belonging to pre-established populations that are likely to suffer from a disease or disorder or characterized at different stages of a disease, wherein said biomarker are consider here as any of the options provided herein.

“Specificity” indicates the performance of the biomarker of the invention with respect to correctly classifying and distinguishing between samples as belonging to pre-established populations of subjects suffering from the same disorder and populations of subjects that are either healthy or not affected by ovarian cancer.

Simply put, “sensitivity” relates to the rate of identification of the patients (samples) as such out of a group of samples, whereas “specificity” relates to the rate of correct identification of ovarian cancer samples as such out of a group of samples. Cutoff values may be used as control sample/s or in addition to control sample/s, said cutoff values being the result of a statistical analysis of biomarker protein expression value/s (specifically the biomarker/s proteins of the invention) differences in pre-established populations healthy or suffering from ovarian cancer, more specifically suffering from high-grade ovarian carcinoma. Pre-established populations as used herein refer to populations of patients diagnosed with ovarian cancer (by any conventional means), or alternatively, populations of healthy subjects.

In yet some further embodiments, a negative or positive determination of the expression value as compared to the predetermined cutoff values, or the expression value of a control sample, also encompass values that are within the range of said cutoff. More specifically, in case the particular biomarker is found to be overexpressed in ovarian cancer, an expression value that is determined by the method of the invention as “positive” when compared to a predetermined cutoff of population of patients suffering from ovarian cancer, or to the expression value of at least one, and preferably, more, known patient/s suffering from ovarian cancer, may indicate that the examined subject belongs to a population suffering from ovarian cancer (e.g., that the subject carries or is affected by ovarian cancer), in case that the expression value is either higher (positive) or fall within the range (the average values of the cutoff predetermined for patient population suffering from ovarian cancer) of the control or standard value. In a similar manner, a subject exhibiting an expression value that is “negative” (that is down-regulated) as compared to the cutoff patients, may be considered as belonging to population that is not suffering from ovarian cancer, in case the expression of the particular biomarker is associated with overexpression in ovarian cancer. In more specific embodiments, the expression value of such subject should fall within the range of the cutoff value predetermined for population that is not suffering from ovarian cancer. In some embodiments, “fall within the range” encompass values that differ from the cutoff value in about 1%, about 5%, about 10%, about 15%, about 20%, about 25%, about 30%, about 35%, about 40%, about 45%, about 50% or more. Simply put, a “positive” expression value as used herein refers to high expression value that reflects overexpression, elevated expression, high expression and even in some embodiments, moderate expression value. A “negative” expression value reflects a repressed, low, reduced, or non-existing expression (lack of expression). Thus, in some embodiments, when a specific biomarker is overexpressed in ovarian cancer, a “positive” expression value of an examined sample may be a value that is higher or within the range of the expression value of a sample taken from a patient affected with ovarian cancer, or a standard cutoff value calculated for ovarian cancer patients. A “negative” value would be an expression value that is lower than the expression value of the ovarian cancer patients (or standard value, or the value of a control sample). Such value may be within the range of the value of a healthy control sample or a standard value of a healthy population of subject, or of subjects that are not affected by ovarian cancer. In yet some further embodiments, when the specific biomarker is associated with low expression or even non-expression (undetectable expression) in ovarian cancer, a “positive” expression value reflects a value that is higher than the value of the ovarian cancer control or standard value. Such value is not within the range of the value of the ovarian cancer population or control sample but may be within the range of the value of the “healthy controls” (as used herein, “healthy controls” may include any subject not affected by ovarian cancer). A “negative” value is meant an expression value that is lower than the expression value of the healthy control that is in that case, within the range of the expression value of ovarian cancer patients.

It should be appreciated that a “control sample” as used herein may reflect a sample of at least one subject (either healthy, a subject that is not affected by ovarian cancer, or alternatively, an ovarian cancer patient), and preferably, a mixture at least two, at least three, at least four, at least five, at least six or more patients.

It should be emphasized that the nature of the invention is such that the accumulation of further patient data may improve the accuracy of the presently provided cutoff values, which are based on an ROC (Receiver Operating Characteristic) curve generated according to said patient data using analytical software program. The biomarker protein expression values are selected along the ROC curve for optimal combination of diagnostic sensitivity and diagnostic specificity which are as close to 100 percent as possible, and the resulting values are used as the cutoff values that distinguish between subjects who are diagnosed with positive OVCA, specifically, HGOC at a certain rate, and those who will not (with said given sensitivity and specificity). Similar analysis may be performed for example when diagnosis of cancer is being examined to distingue between healthy tissue and cancerous tissue. The ROC curve may evolve as more and more data and related biomarker gene expression values are recorded and taken into consideration, modifying the optimal cutoff values and improving sensitivity and specificity. Thus, it should be appreciated that the provided cutoff values should be viewed as a starting point that may shift as more data allows more accurate cutoff value calculation. Although considered as initial cutoff values, the presently provided values already provide good sensitivity and specificity, and are readily applicable in current clinical use, even in patients diagnosed with different cancer stages.

As noted above, the expression value determined for the examined sample (or alternatively, the normalized expression value) is compared with a predetermined cutoff or to a control sample. More specifically, in certain embodiments, the expression value obtained for the examined sample is compared with a predetermined standard or cutoff value.

In further embodiments, the predetermined standard expression value, or cutoff value has been pre-determined and calculated for a population comprising at least one of healthy subjects, subjects suffering from any disorder, subjects suffering from different stages of any disorder, subjects that respond to treatment, non-responder subjects, subjects in remission and subjects in relapse.

Still further, in certain alternative embodiments where a control sample is being used (instead of, or in addition to, pre-determined cutoff values), the expression value or the normalized expression values of the biomarker proteins used by the invention in the test sample are compared to the expression values in the control sample. In certain embodiments, such control sample may be obtained from at least one of a healthy subject, a subject suffering from a disorder at a specific stage, a subject suffering from a disorder at a different specific stage a subject that responds to treatment, a non-responder subject, a subject in remission and a subject in relapse.

It should be appreciated that “Standard” or a “predetermined standard” as used herein, denotes either a single standard value or a plurality of standards with which the level of at least one of the biomarker protein expression from the tested sample is compared. The standards may be provided, for example, in the form of discrete numeric values or is calorimetric in the form of a chart with different colors or shadings for different levels of expression; or they may be provided in the form of a comparative curve prepared on the basis of such standards (standard curve).

It should be noted that for determining the expression value/s of at least one of the biomarker proteins of the invention, the methods of the invention may further comprise the step of providing at least one detecting molecule specific for determining the expression of at least one of the biomarker proteins of the invention. In some embodiments, such detecting molecules may be provided as a mixture, as a composition or as a kit. Thus, in some embodiments, the at least one detecting molecule may be provided as a mixture of detecting molecules, wherein each detecting molecule is specific for one biomarker protein. It should be appreciated however, that for each biomarker protein, one or several specific detecting molecules may be used and provided. In yet some further alternative embodiments, the detecting molecules may be provided separately for each biomarker protein, e.g., in specific tube, containers, slots, spots, wells, and the like. It further alternative embodiments, the detecting molecules may be attached or immobilized to a solid support, specifically, in recorded location.

In some embodiments of the methods disclosed herein, at least one gene associated with high risk for ovarian cancer may be at least one of Breast cancer type 1 susceptibility protein (BRCA1), Breast cancer type 2 susceptibility protein (BRCA2), BRIP1, PALB2, RAD51, BARD1, CHEK2 and MMR genes such as MLH1, MSH2, MSH6, and PMS2. As discussed herein before, the method of the present disclosure may therefore comprise an additional step of determining and/or classifying the diagnosed subjects as carriers or non-carriers of at least one mutation associated with predisposition to cancer, specifically, ovarian cancer. For such classification any appropriate method can be used, for example, an of the methods disclosed in the present disclosure.

In some particular and non-limiting embodiments the methods of the present disclosure involve the step of determining the expression level of at least four biomarker proteins in at least one biological sample of the subject, to obtain an expression value for each of the at least four biomarker protein/s. It should be noted that the at least four proteins are selected from that may be selected from C4BPB, KIF20B, VPS11, CRTAC1, TMEM67, GJA1 and ATP2B4 biomarker proteins. In some particular and non-limiting embodiments, the at least four biomarker proteins may comprise VPS11, ATP2B4, TMEM67 and CRTAC1.

In yet some further embodiments, the methods of the present disclosure involve the step of determining the expression level of all seven biomarker proteins in at least one biological sample of the subject, to obtain an expression value for each of the seven biomarker protein/s, specifically, of the C4BPB, KIF20B, VPS11, CRTAC1, TMEM67, GJA1 and ATP2B4 biomarker proteins, and optionally of at least one additional biomarker protein and/or reference control protein (but no more than 493 additional biomarker protein and/or reference control protein).

Still further, in some specific and non-limiting embodiments, in step (a) of the disclosed method, the expression level of: C4BPB, KIF20B, VPS11, CRTAC1, TMEM67, GJA1 and ATP2B4 biomarker proteins is determined in at least one biological sample of a subject determined as a carrier of at least one mutation in BRCA1 and/or BRCA2, to obtain an expression value for each of the biomarker protein/s.

In yet some alternative or additional embodiments, when model 4 is applied, the expression level of CTSD, SPTBN2, ARFIP1, MIA3, CD109, IRGQ and GOLPH3 biomarker proteins is determined in at least one biological sample of a subject determined as a non-carrier of at least one mutation in BRCA1 and/or BRCA2, to obtain an expression value for each of the biomarker protein/s.

In some embodiments, determination of the level of expression the at least two biomarker protein/s of the present disclosure is performed by quantifying the amount of at least two of the disclosed the C4BPB, KIF20B, VPS11, CRTAC1, TMEM67, GJA1 and ATP2B4 biomarker proteins or of any peptide/s thereof, in a sample.

In yet some alternative embodiments, the determination of the level of expression of at least two of the biomarker protein/s is performed by the step of contacting at least one detecting molecule, or any combination or mixture of plurality of detecting molecules, with at least one biological sample of the diagnosed subject, or with any protein or nucleic acid product obtained therefrom. It should be noted that each of the detecting molecules is specific for one of the biomarker proteins.

Still further, it should be noted that all steps for determining the different parameters indicated above, involve contacting the sample or any component thereof with a specific reagent (e.g., detecting molecules), or any reagent or means required for quantifying the protein amount.

Thus, in yet some specific embodiments, the methods of the invention may involve determining the level of expression of at least one, of at least two, at least three, at least four, at least five, at least six, at least seven of the C4BPB, KIF20B, VPS11, CRTAC1, TMEM67, GJA1 and ATP2B4 biomarkers of the present disclosure by performing the step of contacting at least one detecting molecule or any combination or mixture of plurality of detecting molecules with a biological sample of the subject, or with any protein or nucleic acid product obtained therefrom. It should be noted that each of said detecting molecules is specific for one of the biomarker proteins. In yet some alternative and/or additional embodiments, where model 4 is applicable, the expression level of the CTSD, SPTBN2, ARFIP1, MIA3, CD109, IRGQ and GOLPH3, biomarker protein/s, is determined, as specified above by contacting detecting molecules with the sample.

The term “contacting” means to bring, put, incubates or mix together. As such, a first item is contacted with a second item when the two items are brought or put together, e.g., by touching them to each other or combining them. In the context of the present invention, the term “contacting” includes all measures or steps which allow interaction between the at least one of the detection molecules of at least one of the biomarker proteins, and optionally, for at least one suitable control reference protein of the tested sample. The contacting is performed in a manner so that the at least one of detecting molecule of at least one of the biomarker proteins for example, can interact with or bind to the at least one of the biomarker proteins, in the tested sample. The binding will preferably be non-covalent, reversible binding, e.g., binding via salt bridges, hydrogen bonds, hydrophobic interactions or a combination thereof.

In certain embodiments, the detection step further involves detecting a signal from the detecting molecules that correlates with the expression level of at least one of the biomarker proteins in the sample from the subject, by a suitable means. According to some embodiments, the signal detected from the sample by any one of the experimental methods detailed herein below reflects the expression level of at least one of the biomarker proteins. It should be noted that such signal-to-expression level data may be calculated and derived from a calibration curve.

Thus, in certain embodiments, the method of the invention may optionally further involve the use of a calibration curve created by detecting a signal for each one of the increasing pre-determined concentrations of at least one of the biomarker proteins. Obtaining such a calibration curve may be indicative to evaluate the range at which the expression levels correlate linearly with the concentrations of at least one of the biomarker proteins. It should be noted in this connection that at times when no change in expression level of at least one of the biomarker proteins is observed, the calibration curve should be evaluated in order to rule out the possibility that the measured expression level is not exhibiting a saturation type curve, namely a range at which increasing concentrations exhibit the same signal.

It must be appreciated that in certain embodiments such calibration curve as described above may be also part or component in any of the kits provided by the invention as described herein after.

In yet some further embodiments, the detecting molecule/s may be amino acid detecting molecules and/or nucleic acid detecting molecules.

In more specific embodiments, amino acid detecting molecule/s that may be applicable in the present disclosure may comprise at least one of:

In a first option (a), at least one labeled or tagged protein/s or any fragment/s, peptide/s or mixture/s thereof, of at least one of (i) at least one of C4BPB and KIF20B; (ii) at least one of VPS11, CRTAC1 and TMEM67; (iii) at least one of GJA1 and ATP2B4; and (iv) any combination of at least two biomarker proteins of any one of (i), (ii) and (iii) biomarker proteins.

In some optional embodiments, that may be either in addition to or as an alternative, at least two labeled or tagged protein/s or any fragment/s, peptide/s or mixture/s thereof of at least two of CTSD, SPTBN2, ARFIP1, MIA3, CD109, IRGQ and GOLPH3 biomarker proteins.

In yet some further option (b), the detecting molecules may be at least one antibody specific for at least one of the at least two biomarker proteins disclosed herein.

Still further, in another option (c), the detecting molecules may be at least one protein or peptide aptamer/s specific for at least one of the at least two biomarker proteins.

In yet some further embodiments, the detecting molecules used herein maybe any combination of (a), (b) and (c).

As indicated above, in some embodiments of the invention, the detecting molecules used for determining the expression levels at least one of the biomarker proteins may be selected from isolated detecting amino acid molecules and isolated detecting nucleic acid molecules. It should be noted that the invention further encompasses any combination of nucleic and amino acids for use as detecting molecules for the methods of the invention.

As noted above, in the first step of the method of the invention, the sample or any protein or nucleic acid obtained therefrom, is contacted with the detecting molecules of the invention.

The invention thus contemplates the use of amino acid-based molecules such as proteins or polypeptides as detecting molecules disclosed herein and would be known by a person skilled in the art to measure the at least one biomarker protein. As used herein, the terms “protein” and “polypeptide” are used interchangeably to refer to a chain of amino acids linked together by peptide bonds. In a specific embodiment, a protein is composed of less than 200, less than 175, less than 150, less than 125, less than 100, less than 50, less than 45, less than 40, less than 35, less than 30, less than 25, less than 20, less than 15, less than 10, or less than 5 amino acids linked together by peptide bonds. In another embodiment, a protein is composed of at least 200, at least 250, at least 300, at least 350, at least 400, at least 450, at least 500, at least 1000 or more amino acids linked together by peptide bonds. It should be noted that peptide bond as described herein is a covalent amid bond formed between two amino acid residues. In some embodiments, the detecting molecules used by the methods of the invention may be recombinantly expressed or synthetically prepared. In further embodiments, the recombinantly or synthetically expressed and prepared detecting molecules may be labeled or tagged. It should be noted that in some embodiments, these detecting molecules may be isolated detecting molecules. As used herein, “Recombinant proteins” denotes proteins encoded by a recombinant DNA which is a genetically engineered DNA formed by laboratory methods of genetic recombination to bring together genetic material from multiple sources and thus creating variable sequences. Recombinant proteins may be produced mainly, but not limited, by molecular cloning, namely incorporating the recombinant DNA into a living cell (e.g., bacteria or yeast) and using its system to express the DNA into mRNA and protein thereof.

Techniques for detection and quantification known to persons skilled in the art (for example, Mass spectrometry (MS) or different immunological techniques such as Western Blotting, Immunoprecipitation, ELISAs, protein microarray analysis, Flow cytometry and the like) can then be used to measure the level of protein products corresponding to the biomarker of the invention.

In some embodiments, the term “labeled” or “tagged” may refer to direct labeling of the protein via, e.g., coupling (i.e., physically linking) or incorporating of a detectable substance to the protein. Useful labels in the present invention may include but are not limited to include isotopes (e.g. ¹³C, ¹⁵N), or any other radiolabels (e.g., ³H, ¹²⁵I, ³⁵S, ¹⁴C, or ³²P), magnetic beads (e.g. DYNABEADS), fluorescent dyes (e.g., fluorescein isothiocyanate, Texas red, rhodamine, green fluorescent protein, and the like), enzymes (e.g., horseradish peroxidase, alkaline phosphatase and others commonly used in an ELISA and competitive ELISA, histochemistry and other similar methods known in the art) and colorimetric labels such as colloidal gold or colored glass or plastic (e.g. polystyrene, polypropylene, latex, etc.) beads. In some embodiments, the protein may be tagged. Different tags may be also used, for example, His, myc, HA, GFP, ABP, GST, biotin and the like. “tagged” as used herein may further include fusion or linking of the biomarker protein or any fragment or peptide thereof, that serves herein as a detecting molecule, a tag that in some embodiments may contain several amino acids or a peptide that may be recognized by affinity or immunologically, using specific antibodies.

In some other embodiments, the biomarker proteins or any fragments or peptides thereof may be fluorescently labeled. In another embodiment, the biomarker proteins or any fragments or peptides thereof may be isotope labeled. The term “recombinant isotope labeled” denotes a protein ‘labeled’ by replacing specific atoms by their isotope.

Means of detecting such labels are well known to those of skill in the art. Thus, for example, radiolabels may be detected using photographic film or scintillation counters, fluorescent markers may be detected using a photodetector to detect emitted illumination. Enzymatic labels are typically detected by providing the enzyme with a substrate and detecting the reaction product produced by the action of the enzyme on the substrate, and colorimetric labels are detected by simply visualizing the colored label.

More specifically, in certain embodiments the biomarker proteins of the invention or any fragment or peptide thereof, when recombinantly expressed and labeled or tagged, may be used as detecting molecules for determining the quantity or level of expression of the biomarker proteins of the invention in the examined sample. The term “labeled form” as used herein includes an isotope labeled form. Specifically, the labeled form is a chemically or metabolically isotope labeled, and more specifically a metabolically isotope labeled form of the biomarker proteins of the invention.

Optional “isotope labeled forms” of the biomarker protein/s or any fragments or peptides thereof in accordance with the present invention are variants of naturally occurring molecules, in whose structure one or more atoms have been substituted with atom(s) of the same element having a different atomic weight, although isotope labeled forms in which the isotope has been covalently linked either directly or via a linker, or wherein the isotope has been complexed to the biomarker proteins are likewise contemplated. In either case, the isotope may be stable isotope.

A stable isotope as referred to herein, is a non-radioactive isotopic form of an element having identical numbers of protons and electrons, but having one or more additional neutron(s), which increase(s) the molecular weight of the element. Specifically, the stable isotopes may be selected from the group consisting of ²H, ¹³C, ¹⁵N, ¹⁷O, ¹⁸O, ³³P, ³⁴S and combinations thereof. Particularly specific examples include ¹³C and ⁵N, and combinations thereof.

The labeling can be affected by means known in the art. A labeled reference biomarker (used as detecting molecule) can be synthesized using isotope labeled amino acids as precursor molecules, or chemically modified. Modification and labeling can be done on whole proteins or their fragments. For example, isotope-coded affinity tag (ICAT) reagents label reference biomolecule such as proteins at the alkylation step of sample preparation (WO2004079370). Visible ICAT reagents (VICAT reagents) may be likewise employed (WO2011042467), whereby the VICAT-type reagent contains as a detectable moiety a fluorophore or radiolabel. iTRAQ, TMT and similar methods may likewise be employed.

Metabolic labeling may also be used to produce the labeled reference biomarkers. For example, cells can be grown on media containing isotope labeled precursor molecules, such as isotope labeled amino acids, that are incorporated into proteins or peptides, which are thereby metabolically labeled. The metabolic isotope labeling may be a stable isotope labeling with amino acids in cell culture (SILAC). If metabolic labeling is used, and the labeled form of the one or the plurality of reference biomarker protein/s is a SILAC labeled form of the reference biomarker protein/s, the standard mixture as defined above is also referred to as SUPER-SILAC mix.

In some embodiments, the detecting molecules may be affinity molecules such as antibodies, or aptamers. In some embodiments, the detecting molecules may be antibodies and the determination of the level of expression of the at least two of the biomarker proteins of the present disclosure may be performed by any appropriate immuno assay.

It should be understood that each antibody specifically recognizes one biomarker protein. Using these antibodies, the level of expression of at least one of the biomarker protein may be determined using an immunoassay which may be an assay that includes but not limited to FACS, a Western blot, an ELISA, a RIA, a slot blot, a dot blot, immune-histochemical assay and a radio-imaging assay, as described in more detail herein after. It should be noted that such assay may be performed using microarray protein arrays.

More specifically, the term “antibody” as used in this invention includes whole antibody molecules as well as functional fragments thereof, such as Fab, F(ab′)2, and Fv that are capable of binding with antigenic portions of the target polypeptide, i.e., at least one of the biomarker proteins. The antibody may be preferably monospecific, e.g., a monoclonal antibody, or antigen-binding fragment thereof. The term “monospecific antibody” refers to an antibody that displays a single binding specificity and affinity for a particular target, e.g., epitope. This term includes a “monoclonal antibody” or “monoclonal antibody composition”, which, as used herein, refer to a preparation of antibodies or fragments thereof of single molecular composition.

It should be recognized that the antibody can be a human antibody, a chimeric antibody, a recombinant antibody, a humanized antibody, a monoclonal antibody, or a polyclonal antibody. The antibody can be an intact immuno globulin, e.g., an IgA, IgG, IgE, IgD, IgM or subtypes thereof. The antibody can be conjugated to a labeling moiety as discussed above.

As noted above, the term “antibody” also encompasses antigen-binding fragments of an antibody. The term “antigen-binding fragment” of an antibody (or simply “antibody portion,” or “fragment”), as used herein, may be defined as follows:
(1) Fab, the fragment which contains a monovalent antigen-binding fragment of an antibody molecule, can be produced by digestion of whole antibody with the enzyme papain to yield an intact light chain and a portion of one heavy chain;
(2) Fab′, the fragment of an antibody molecule that can be obtained by treating whole antibody with pepsin, followed by reduction, to yield an intact light chain and a portion of the heavy chain; two Fab′ fragments are obtained per antibody molecule;
(3) (Fab′)2, the fragment of the antibody that can be obtained by treating whole antibody with the enzyme pepsin without subsequent reduction; F(ab′)2 is a dimer of two Fab′ fragments held together by two disulfide bonds;
(4) Fv, defined as a genetically engineered fragment containing the variable region of the light chain and the variable region of the heavy chain expressed as two chains; and
(5) Single chain antibody (“SCA”, or ScFv), a genetically engineered molecule containing the variable region of the light chain and the variable region of the heavy chain, linked by a suitable polypeptide linker as a genetically fused single chain molecule.

Methods of generating such antibody fragments are well known in the art (See for example, Harlow and Lane, Antibodies: A Laboratory Manual, Cold Spring Harbor Laboratory, New York, 1988, incorporated herein by reference).

Purification of serum immunoglobulin antibodies (polyclonal antisera) or reactive portions thereof can be accomplished by a variety of methods known to those of skill in the art including, precipitation by ammonium sulfate or sodium sulfate followed by dialysis against saline, ion exchange chromatography, affinity or immuno-affinity chromatography as well as gel filtration, zone electrophoresis, etc.

Still further, the antibodies used by the present invention may optionally be covalently or non-covalently linked to a detectable label or tag. In addition, the label and can also refer to indirect labeling of the protein by reactivity with another reagent that is directly labeled.

Examples of indirect labeling include detection of at least one of the biomarker protein/s of the invention using a fluorescently labeled secondary antibody. More specifically, detectable labels suitable for such use include any composition detectable by spectroscopic, photochemical, biochemical, immunochemical, electrical, optical or chemical means.

The antibody used as a detecting molecule according to the invention, specifically recognizes and binds at least one of the biomarker proteins. It should be noted that in certain embodiments, each antibody is specific for one of the biomarker proteins of the invention, specifically, those disclosed by the present disclosure, specifically, any one of C4BPB, KIF20B, VPS11, CRTAC1, TMEM67, GJA1 and ATP2B4 biomarker proteins.

It should be appreciated that antibodies that may be used by the methods as well as the compositions and kits of the invention, may be antibodies directed not only against the biomarker proteins of the invention, but also in case the biomarkers are tagged, the antibodies may be directed against said tags. It should be therefore noted that the term “binding specificity”, “specifically binds to an antigen”, “specifically immuno-reactive with”, “specifically directed against” or “specifically recognizes”, when referring to an epitope, specifically, a recognized epitope within the at least one of the biomarker protein, refers to a binding reaction which is determinative of the presence of the epitope in a heterogeneous population of proteins and other biologics. More particularly, “selectively bind” in the context of proteins encompassed by the invention refers to the specific interaction of any two of a peptide, a protein, a polypeptide an antibody, wherein the interaction preferentially occurs as between any two of a peptide, protein, polypeptide and antibody preferentially as compared with any other peptide, protein, polypeptide and antibody.

Thus, under designated immunoassay conditions, the specified antibodies bind to a particular epitope at least two times the background and more typically more than 10 to 100 times background. More specifically, “Selective binding”, as the term is used herein, means that a molecule binds its specific binding partner with at least 2-fold greater affinity, and preferably at least 10-fold, 20-fold, 50-fold, 100-fold or higher affinity than it binds a non-specific molecule.

It should be appreciated that the antibodies used by the methods of the invention, may be in some embodiments antibodies that are not naturally occurring antibodies. More specifically, the antibodies are not produced naturally in the body, and more specifically, it should be appreciated that production thereof involves immunological and recombinant techniques.

A variety of immunoassay formats may be used to select antibodies specifically immuno-reactive with a particular protein or carbohydrate. For example, solid-phase ELISA immunoassays are routinely used to select antibodies specifically immuno-reactive with a protein or carbohydrate. The term “epitope” is meant to refer to that portion of any molecule capable of being bound by an antibody which can also be recognized by that antibody. Epitopes or “antigenic determinants” usually consist of chemically active surface groupings of molecules such as amino acids or sugar side chains and have specific three-dimensional structural characteristics as well as specific charge characteristics.

In some other embodiments, the detecting molecules are peptide aptamers specific for said at least one of said biomarker proteins. “Peptide or protein aptamers” as used herein refers to small peptides with a single variable loop region tied to a protein scaffold on both ends that binds to a specific molecular target (e.g. protein), and which are bind to their targets only with said variable loop region and usually with high specificity properties.

According to one embodiment, where amino acid-based detection molecules are used, the expression level of the at least one of the biomarker proteins, in the tested sample can be determined using different methods known in the art, specifically method disclosed herein below as non-limiting examples.

In some alternative embodiments, determination of the expression levels of the biomarker proteins of the invention may be performed in the nucleic acid level, specifically, the mRNA level. In such embodiments for determining the expression level of the biomarkers of the invention, nucleic acid detecting molecule may be used. Thus, in certain embodiments, the methods disclosed herein may use a nucleic acid detecting molecule/s.

According to such embodiments, the nucleic acid detecting molecules may comprise at least one of: In one option (a), at least one nucleic acid aptamer/s specific for the at least one of the at least two biomarker proteins. In yet some further option (b), the detecting molecule may be at least one oligonucleotide/s. In some embodiments, each oligonucleotide specifically hybridizes to a nucleic acid sequence encoding at least one of the at least two biomarker proteins.

In some embodiments, the nucleic acid detecting molecule/s of the invention may comprise at least one of: (a) nucleic acid aptamers specific for the at least one of the biomarker proteins; and (b) at least one isolated oligonucleotides, each oligonucleotide specifically hybridizes to a nucleic acid sequence encoding the at least one biomarker protein.

As used herein, “nucleic acid molecules” or “nucleic acid sequence” are interchangeable with the term “polynucleotide(s)” and it generally refers to any polyribonucleotide or poly-deoxyribonucleotide, which may be unmodified RNA or DNA or modified RNA or DNA or any combination thereof. “Nucleic acids” include, without limitation, single- and double-stranded nucleic acids. As used herein, the term “nucleic acid(s)” also includes DNAs or RNAs as described above that contain one or more modified bases. Thus, DNAs or RNAs with backbones modified for stability or for other reasons are “nucleic acids”. The term “nucleic acids” as it is used herein embraces such chemically, enzymatically or metabolically modified forms of nucleic acids, as well as the chemical forms of DNA and RNA characteristic of viruses and cells, including for example, simple and complex cells. A “nucleic acid” or “nucleic acid sequence” may also include regions of single- or double-stranded RNA or DNA or any combinations.

More specifically, in some other embodiments, the nucleic acid detecting molecules may comprise at least one isolated oligonucleotide/s, each oligonucleotide specifically hybridizes to a nucleic acid sequence encoding one of said at least one biomarker protein.

In an optional embodiment, where the expression levels of the biomarkers of the invention are normalized, the method of the invention may use nucleic acid detecting molecules specific for a nucleic acid sequence encoding the control reference protein/s.

As used herein, the term “oligonucleotide” is defined as a molecule comprised of two or more deoxyribonucleotides and/or ribonucleotides, and preferably more than three. Its exact size will depend upon many factors which in turn, depend upon the ultimate function and use of the oligonucleotide. The oligonucleotides may be from about 3 to about 1,000 nucleotides long. Although oligonucleotides of 5 to 100 nucleotides are useful in the invention, preferred oligonucleotides range from about 5 to about 15 bases in length, from about 5 to about 20 bases in length, from about 5 to about 25 bases in length, from about 5 to about 30 bases in length, from about 5 to about 40 bases in length or from about 5 to about 50 bases in length. More specifically, the detecting oligonucleotides molecule used by the composition of the invention may comprise any one of 5,6,7, 8,9, 10, 11, 12, 13, 14, 15, 16, 17, 18, 19, 20, 21, 22, 23, 24, 25, 26, 27, 28, 29, 30, 35, 40, 45, 50 bases in length. It should be further noted that the term “oligonucleotide” refers to a single stranded or double stranded oligomer or polymer of ribonucleic acid (RNA) or deoxyribonucleic acid (DNA) or mimetics thereof. This term includes oligonucleotides composed of naturally-occurring bases, sugars and covalent internucleoside linkages (e.g., backbone) as well as oligonucleotides having non-naturally-occurring portions which function similarly.

In some embodiments, nucleic acid based detecting molecules may involve the step of hybridization of the detecting molecule/s (e.g. the prob and/or primer specific for each of the biomarkers of the present disclosure) with nucleic acids of the examined sample.

The term “hybridize” or “Hybridization”, as used herein is the process in which two complementary single-stranded DNA and/or RNA molecules bond together to form a double-stranded molecule. The bonding is dependent on the appropriate base-pairing across the two single-stranded molecules.

In yet some further specific embodiments, where the detecting molecules of the invention are nucleic acid-based molecules, optional detecting molecule/s may be at least one nucleic acid aptamer specific for the at least one of said biomarker proteins.

As used herein the term “aptamer” or “specific aptamers” denotes single-stranded nucleic acid (DNA or RNA) molecules which specifically recognizes and binds to a target molecule. The aptamers according to the invention may fold into a defined tertiary structure and can bind a specific target molecule with high specificities and affinities. Aptamers are usually obtained by selection from a large random sequence library, using methods well known in the art, such as SELEX and/or Molinex. In various embodiments, aptamers may include single-stranded, partially single-stranded, partially double-stranded or double-stranded nucleic acid sequences; sequences comprising nucleotides, ribonucleotides, deoxyribonucleotides, nucleotide analogs, modified nucleotides and nucleotides comprising backbone modifications, branch points and non-nucleotide residues, groups or bridges; synthetic RNA, DNA and chimeric nucleotides, hybrids, duplexes, heteroduplexes; and any ribonucleotide, deoxyribonucleotide or chimeric counterpart thereof and/or corresponding complementary sequence. In certain specific embodiments, aptamers used by the invention are composed of deoxyribonucleotides.

According to the present invention and as appreciated in the art, the recognition between the aptamer and the antigen is specific and may be detected by the appearance of a detectable signal by using a colorimetric sensor or a fluorimetric/lumination sensor, radioactive sensor, or any appropriate means.

The aptamers that may be used according to some aspects of the invention may be biotinylated. The aptamers may optionally include a chemically reactive group at the 3′ and/or 5′ termini. The term reactive group is used herein to denote any functional group comprising a group of atoms which is found in a molecule and is involved in chemical reactions. Some non-limiting examples for a reactive group include primary amines (NH₂), thiol (SH), carboxy group (COOH), phosphates (PO4), Tosyl, and a photo-reactive group.

In some embodiments, the aptamer that may be applicable herein may optionally comprise a spacer between the nucleic acid sequence and the reactive group. The spacer may be an alkyl chain such as (CH₂)_{6/12}, namely comprising six to twelve carbon atoms.

In yet some other alternative embodiments, the detection molecule may be at least one primer, at least one pair of primers, nucleotide probes and any combinations thereof.

Thus, it should be further appreciated that the methods, as well as the compositions and kits of the invention may comprise, as an oligonucleotide-based detection molecule, both primers and probes.

The term, “primer”, as used herein refers to an oligonucleotide, whether occurring naturally as in a purified restriction digest, or produced synthetically, which is capable of acting as a point of initiation of synthesis when placed under conditions in which synthesis of a primer extension product, which is complementary to a nucleic acid strand, is induced, i.e., in the presence of nucleotides and an inducing agent such as a DNA polymerase and at a suitable temperature and pH. The primer may be single-stranded or double-stranded and must be sufficiently long to prime the synthesis of the desired extension product in the presence of the inducing agent. The exact length of the primer will depend upon many factors, including temperature, source of primer and the method used. For example, for diagnostic applications, depending on the complexity of the target sequence, the oligonucleotide primer typically contains 10-30 or more nucleotides, although it may contain fewer nucleotides. More specifically, the primer used by the methods, as well as the compositions and kits of the invention may comprise 10, 11, 12, 13, 14, 15, 16, 17, 18, 19, 20, 21, 22, 23, 24, 25, 26, 27, 28, 29 or 30 nucleotides or more.

In certain embodiments, such primers may comprise 30, 40, 50, 60, 70, 80, 90, 100 nucleotides or more. In specific embodiments, the primers used by the method of the invention may have a stem and loop structure. The factors involved in determining the appropriate length of primer are known to one of ordinary skill in the art and information regarding them is readily available.

As used herein, the term “probe” means oligonucleotides and analogs thereof and refers to a range of chemical species that recognize polynucleotide target sequences through hydrogen bonding interactions with the nucleotide bases of the target sequences. The probe or the target sequences may be single- or double-stranded RNA or single- or double-stranded DNA or a combination of DNA and RNA bases. A probe may be 5, 6, 7,8,9, 10, 11, 12, 13, 14, 15, 16, 17, 18, 19,20,21,22,23,24,25,26,27,28,29 and up to 30 or more nucleotides in length as long as it is less than the full length of the target mRNA or any gene encoding said mRNA. Probes can include oligonucleotides modified so as to have a tag which is detectable by fluorescence, chemiluminescence and the like.

The probe can also be modified so as to have both a detectable tag and a quencher molecule, for example TaqMan(R) and Molecular Beacon(R) probes.

The oligonucleotides and analogs thereof may be RNA or DNA, or analogs of RNA or DNA, commonly referred to as antisense oligomers or antisense oligonucleotides. Such RNA or DNA analogs comprise, but are not limited to, 2-′0-alkyl sugar modifications, methylphosphonate, phosphorothiate, phosphorodithioate, formacetal, 3-thioformacetal, sulfone, sulfamate, and nitroxide backbone modifications, and analogs, for example, LNA analogs, wherein the base moieties have been modified. In addition, analogs of oligomers may be polymers in which the sugar moiety has been modified or replaced by another suitable moiety, resulting in polymers which include, but are not limited to, morpholino analogs and peptide nucleic acid (PNA) analogs. Probes may also be mixtures of any of the oligonucleotide analog types together or in combination with native DNA or RNA. At the same time, the oligonucleotides and analogs thereof may be used alone or in combination with one or more additional oligonucleotides or analogs thereof.

According to this option, the expression level may be determined using amplification assay. The term “amplification assay”, with respect to nucleic acid sequences, refers to methods that increase the representation of a population of nucleic acid sequences in a sample. Nucleic acid amplification methods, such as PCR, isothermal methods, rolling circle methods, etc., are well known to the skilled artisan. More specifically, as used herein, the term “amplified”, when applied to a nucleic acid sequence, refers to a process whereby one or more copies of a particular nucleic acid sequence is generated from a template nucleic acid, preferably by the method of polymerase chain reaction.

“Polymerase chain reaction” or “PCR” refers to an in vitro method for amplifying a specific nucleic acid template sequence. The PCR reaction involves a repetitive series of temperature cycles and is typically performed in a volume of 50-100 microliter. The reaction mix comprises dNTPs (each of the four deoxynucleotides dATP, dCTP, dGTP, and dTTP), primers, buffers, DNA polymerase, and nucleic acid template. The PCR reaction comprises providing a set of polynucleotide primers wherein a first primer contains a sequence complementary to a region in one strand of the nucleic acid template sequence and primes the synthesis of a complementary DNA strand, and a second primer contains a sequence complementary to a region in a second strand of the target nucleic acid sequence and primes the synthesis of a complementary DNA strand, and amplifying the nucleic acid template sequence employing a nucleic acid polymerase as a template-dependent polymerizing agent under conditions which are permissive for PCR cycling steps of (i) annealing of primers required for amplification to a target nucleic acid sequence contained within the template sequence, (ii) extending the primers wherein the nucleic acid polymerase synthesizes a primer extension product. “A set of polynucleotide primers”, “a set of PCR primers” or “pair of primers” can comprise two, three, four or more primers.

Real time nucleic acid amplification and detection methods are efficient for sequence identification and quantification of a target since no pre-hybridization amplification is required. Amplification and hybridization are combined in a single step and can be performed in a fully automated, large-scale, closed-tube format.

Methods that use hybridization-triggered fluorescent probes for real time PCR are based either on a quench-release fluorescence of a probe digested by DNA Polymerase (e.g., methods using TaqMan(R), MGB-TaqMan(R)), or on a hybridization-triggered fluorescence of intact probes (e.g., molecular beacons, and linear probes). In general, the probes are designed to hybridize to an internal region of a PCR product during annealing stage (also referred to as amplicon). For those methods utilizing TaqMan(R) and MGB-TaqMan(R) the 5′-exonuclease activity of the approaching DNA Polymerase cleaves a probe between a fluorophore and a quencher, releasing fluorescence.

Thus, a “real time PCR” or “RT-PCT” assay provides dynamic fluorescence detection of amplified biomarker proteins of the present disclosure, or any control reference gene produced in a PCR amplification reaction. During PCR, the amplified products created using suitable primers hybridize to probe nucleic acids (TaqMan(R) probe, for example), which may be labeled according to some embodiments with both a reporter dye and a quencher dye. When these two dyes are in close proximity, i.e. both are present in an intact probe oligonucleotide, the fluorescence of the reporter dye is suppressed. However, a polymerase, such as AmpliTaq Gold™, having 5′-3′ nuclease activity can be provided in the PCR reaction. This enzyme cleaves the fluorogenic probe if it is bound specifically to the target nucleic acid sequences between the priming sites. The reporter dye and quencher dye are separated upon cleavage, permitting fluorescent detection of the reporter dye. Upon excitation by a laser provided, e.g., by a sequencing apparatus, the fluorescent signal produced by the reporter dye is detected and/or quantified. The increase in fluorescence is a direct consequence of amplification of target nucleic acids during PCR. More particularly, QRT-PCR or “qPCR” (Quantitative RT-PCR), which is quantitative in nature, can also be performed to provide a quantitative measure of gene expression levels. In QRT-PCR reverse transcription and PCR can be performed in two steps, or reverse transcription combined with PCR can be performed. One of these techniques, for which there are commercially available kits such as TaqMan(R) (Perkin Elmer, Foster City, CA), is performed with a transcript-specific antisense probe. This probe is specific for the PCR product (e.g. a nucleic acid fragment derived from a gene) and is prepared with a quencher and fluorescent reporter probe attached to the 5′ end of the oligonucleotide.

Different fluorescent markers are attached to different reporters, allowing for measurement of at least two products in one reaction.

When Taq DNA polymerase is activated, it cleaves off the fluorescent reporters of the probe bound to the template by virtue of its 5-to-3′ exonuclease activity. In the absence of the quenchers, the reporters now fluoresce. The color change in the reporters is proportional to the amount of each specific product and is measured by a fluorometer; therefore, the amount of each color is measured, and the PCR product is quantified. The PCR reactions can be performed in any solid support, for example, slides, microplates, 96 well plates, 384 well plates and the like so that samples derived from many individuals are processed and measured simultaneously. The TaqMan(R) system has the additional advantage of not requiring gel electrophoresis and allows for quantification when used with a standard curve.

A second technique useful for detecting PCR products quantitatively without is to use an intercalating dye such as the commercially available QuantiTect SYBR Green PCR (Qiagen, Valencia California). RT-PCR is performed using SYBR green as a fluorescent label which is incorporated into the PCR product during the PCR stage and produces fluorescence proportional to the amount of PCR product.

Both TaqMan(R) and QuantiTect SYBR systems can be used subsequent to reverse transcription of RNA. Reverse transcription can either be performed in the same reaction mixture as the PCR step (one-step protocol) or reverse transcription can be performed first prior to amplification utilizing PCR (two-step protocol).

Additionally, other known systems to quantitatively measure mRNA expression products include Molecular Beacons(R) which uses a probe having a fluorescent molecule and a quencher molecule, the probe capable of forming a hairpin structure such that when in the hairpin form, the fluorescence molecule is quenched, and when hybridized, the fluorescence increases giving a quantitative measurement of gene expression.

According to this embodiment, the detecting molecule may be in the form of probe corresponding and thereby hybridizing to any region or at least one of the biomarker protein or any control reference protein. More particularly, it is important to choose regions which will permit hybridization to the target nucleic acids. Factors such as the Tm of the oligonucleotide, the percent GC content, the degree of secondary structure and the length of nucleic acid are important factors.

It should be noted however that a standard Northern blot assay or dot blot can also be used to ascertain an RNA transcript size and the relative amounts of the biomarker proteins of the invention or any control gene product, in accordance with conventional Northern hybridization techniques known to those persons of ordinary skill in the art.

Still further, in some embodiments, the detecting molecule/s are at least two labeled or tagged protein/s or any fragment/s, peptide/s or mixture/s thereof, of at least two biomarker proteins of at least one of: (i) least one of C4BPB and KIF20B; (ii) least one of VPS11, CRTAC1 and TMEM67; (iii) least one of GJA1 and ATP2B4; and (iv) any combination of at least two biomarker proteins of any one of (i), (ii) and (iii), protein/s or any fragment/s, peptide/s or mixture/s thereof.

In some optional embodiments, that may be either in addition to, or as an alternative, at least two labeled or tagged protein/s or any fragment/s, peptide/s or mixture/s thereof of at least two of CTSD, SPTBN2, ARFIP1, MIA3, CD109, IRGQ and GOLPH3 protein/s or any fragment/s, peptide/s or mixture/s thereof, may be provided. In some specific embodiments, the determination of the expression level of the at least one biomarker protein/s is performed by mass spectrometry.

Mass spectrometry (MS) is used herein as an analytical chemistry technique to identify the amount and type of chemicals present in a sample by measuring the mass-to-charge ratio and abundance of gas-phase ions. A mass spectrum is a plot of the ion signal as a function of the mass-to-charge ratio. The spectra are used to determine the elemental or isotopic signature of a sample, the masses of particles and of molecules, and to elucidate the chemical structures of molecules, such as peptides and other chemical compounds.

As noted above, the invention contemplates the use of Mass spectrometry-based absolute quantification assays that generally require recombinant expression of full length, labeled protein standards. Mass spectrometry is not inherently quantitative, but many methods have been developed to overcome this limitation. Most of them are based on stable isotopes and introduce a mass shifted version of the peptides of interest, which are then quantified by their “heavy” to “light” ratio. Stable isotope labeling is either accomplished by chemical addition of labeled reagents, enzymatic isotope labeling, or metabolic labeling. Generally, these approaches are used to obtain relative quantitative information on protein expression levels in a light and a heavy labeled sample. For example, stable isotope labeling by amino acids in cell culture (SILAC) is performed by metabolic incorporation of light or heavy labeled amino acids into the recombinant or synthetic protein. Labeled protein can also be used as internal standards for determining expression levels of a cell or tissue protein of interest, such as in the spike-in SILAC approach.

Several methods for absolute quantification have emerged over the last years and may be applicable for the present invention, including absolute quantification (AQUA), quantification concatamer (QConCAT), protein standard absolute quantification (PSAQ), absolute SILAC, and FlexiQuant. They all quantify the endogenous protein of interest by the heavy to light ratios to a defined amount of the labeled counterpart spiked into the sample and are chiefly distinguished by either spiking in heavy labeled peptides or heavy labeled full-length proteins. The AQUA strategy is convenient and streamlined: proteotypic peptides are chemically synthesized with heavy isotopes and spiked in after sample preparation.

Still further, the QconCAT approach is based on artificial proteins that are concatamers of proteotypic peptides. This artificial protein is recombinantly expressed in host cells, for example, bacterial cells such as Escherichia coli and spiked into the sample before proteolysis. QconCAT in principle allows efficient production of labeled peptides but does not automatically correct for protein fractionation effects or digestion efficiency in the native proteins versus the concatamers. The PSAQ, absolute SILAC and FexiQuant approaches sidestep these limitations by metabolically labeling full length proteins by heavy versions of the amino acid arginine and lysine. PSAQ and FlexiQuant in vitro synthesize full-length proteins in wheat germ extracts or in bacterial cell extract, respectively, whereas absolute SILAC was described with recombinant protein expression in E. coli. The protein standard is added at an early stage, such as directly to cell lysate. Consequently, sample fractionation can be performed in parallel and the SILAC protein is digested together with the proteome under investigation. Another quantitative approach applicable for the purpose of the present invention may be in some embodiments the SILAC-PrEST assay. In this method, Protein Epitope Signature Tags (PrESTs) are expressed recombinantly in E. coli and they consist of a short and unique region of the protein of interest as well as purification and solubility tags. A highly purified, stable isotope labeling of amino acids in cell culture (SILAC)-labeled version of the solubility tag is first quantified and used to determine the precise amount of each PrEST by its SILAC ratios. The PrESTs are then spiked into the examined sample (e.g., cell lysates) and the SILAC ratios of PrEST peptides to peptides from endogenous target proteins yield their cellular quantities.

In some embodiments, in the context of the present invention, the labeled or tagged biomarker/s of the invention or any labeled fragments or peptides thereof (that are used herein as detecting molecules) are mixed with the sample of with any protein extracted therefrom. The resulting protein mixture may be then digested according to the FASP protocol [Wisniewski, J. R et al., Nat Meth 6:359-362(2009)] and the peptides are separated into fractions by anion exchange chromatography in a StageTip format [Wisniewski al., Journal of Proteome Research 8:5674-5678 (2009)]. Each fraction is analyzed by online reverse-phase chromatography coupled to high resolution, quantitative mass spectrometry analysis.

A variety of mass spectrometry systems can be employed in the methods of the invention for identifying and/or quantifying a biomarker protein of the invention or any fragment or peptide thereof in a sample. Mass analyzers with high mass accuracy, high sensitivity and high resolution include, but are not limited to, Q-Exative Plus, Q-Exactive HF, Exploris or Eclipse mass spectrometers (ThermoFischer scientific), matrix-assisted laser desorption time-of-flight (MALDI-TOF) mass spectrometers, electrospray ionization time-of-flight (ESI-TOF) mass spectrometers and Fourier transform ion cyclotron mass analyzers (FT-ICR-MS) instruments. Other modes of MS include ion trap and triple quadrupole mass spectrometers. In ion trap MS, analytes are ionized by electrospray ionization or MALDI and then put into an ion trap. Trapped ions can then be separately analyzed by MS upon selective release from the ion trap. Ion traps can also be combined with the other types of mass spectrometers described above.

Fragments can also be generated and analyzed. Reference biomarker protein/s labeled with an ICAT or VICAT or iTRAQ or TMT type reagent, or SILAC labeled peptides can be analyzed, for example, by single stage mass spectrometry with a MALDI or ESI ionization and with TOF, quadrupole, iontrap, FT-ICR or Orbitrap analyzers. Methods of mass spectrometry analysis are well known to those skilled in the art. For high resolution peptide fragment separation, liquid chromatography ESI-MS/MS or automated LC-MS/MS, can be used. MS analysis can be performed in a data-dependent manner, data-independent manner, or using targeted MS techniques such as selected reaction monitoring (SRM) or parallel reaction monitoring (PRM).

In some other embodiments, when the detecting molecules used are at least one of antibodies, nucleic acid, peptide or protein aptamers or any combination thereof, specific for the at least two of the biomarker proteins, the determination of the expression level of said biomarker protein/s may be performed by an immunological assay.

Thus, in some alternative embodiments, the detecting molecule/s may be at least one of antibodies, peptide or protein and aptamer/s specific for the at least two of the biomarker protein/s, or any combination thereof. According to such embodiments, the determination of the expression level of the at least one biomarker protein/s is performed by an immunological assay.

In some specific embodiments, determination of the expression level of the biomarker may be performed using ELISA. Enzyme-Linked Immunosorbent Assay (ELISA) is used herein involves fixation of a sample containing a protein substrate (e.g., fixed cells or a protein solution) to a surface such as a well of a microtiter plate. A substrate-specific antibody coupled to an enzyme is applied and allowed to bind to the substrate. Presence of the antibody is then detected and quantitated by a colorimetric reaction employing the enzyme coupled to the antibody. Enzymes commonly employed in this method include horseradish peroxidase and alkaline phosphatase. If well calibrated and within the linear range of response, the amount of substrate present in the sample is proportional to the amount of color produced. A substrate standard is generally employed to improve quantitative accuracy.

In some specific embodiments, determination of the expression level of the biomarker may be performed using Western blot. Western Blot as used herein involves separation of a substrate from other protein by means of an acryl amide gel followed by transfer of the substrate to a membrane (e.g., nitrocellulose, nylon, or PVDF). Presence of the substrate is then detected by antibodies specific to the substrate, which are in turn detected by antibody-binding reagents. Antibody-binding reagents may be, for example, protein A or secondary antibodies. Antibody-binding reagents may be radio labeled or enzyme-linked, as described hereinafter. Detection may be by autoradiography, colorimetric reaction, or chemiluminescence. This method allows both quantization of an amount of substrate and determination of its identity by a relative position on the membrane indicative of the protein's migration distance in the acryl amide gel during electrophoresis, resulting from the size and other characteristics of the protein.

In some specific embodiments, different RIA assays may be employed for determination of the expression level of the biomarker proteins of the invention. In one version, Radioimmunoassay (RIA) involves precipitation of the desired protein (i.e., the substrate) with a specific antibody and radio labeled antibody-binding protein (e.g., protein A labeled with I¹²⁵) immobilized on a perceptible carrier such as agars beads. The radio-signal detected in the precipitated pellet is proportional to the amount of substrate bound. In an alternate version of RIA, a labeled substrate and an unlabeled antibody-binding protein are employed. A sample containing an unknown amount of substrate is added in varying amounts. The number of radio counts from the labeled substrate-bound precipitated pellet is proportional to the amount of substrate in the added sample.

Still further, in specific embodiments, determination of the expression level of the biomarker/s of the invention may be performed using FACS. Fluorescence-Activated Cell Sorting (FACS) involves detection of a substrate in situ in cells bound by substrate-specific, fluorescently labeled antibodies. The substrate-specific antibodies are linked to fluorophore. Detection is by means of a flow cytometry machine, which reads the wavelength of light emitted from each cell as it passes through a light beam. This method may employ two or more antibodies simultaneously and is a reliable and reproducible procedure used by the present invention.

In some specific embodiments, determination of the expression level of the biomarker may be performed using immunohistochemistry methods. Immuno histochemical Analysis involves detection of a substrate in situ in fixed cells by substrate-specific antibodies. The substrate specific antibodies may be enzyme-linked or linked to fluorophore. Detection is by microscopy and is either subjective or by automatic evaluation. With enzyme-linked antibodies, a calorimetric reaction may be required. It will be appreciated that immunohistochemistry is often followed by counterstaining of the cell nuclei, using, for example, Hematoxyline or Giemsa stain.

It should be appreciated that all the detecting molecules used by any of the methods, as well as the compositions and kits of the invention described herein after, are isolated and/or purified molecules. As used herein, “isolated” or “purified” when used in reference to a nucleic acid (probes, primers and aptamers) means that a naturally occurring sequence has been removed from its normal cellular environment or is synthesized in a non-natural environment (e.g., artificially synthesized). Thus, an “isolated” or “purified” sequence may be in a cell-free solution or placed in a different cellular environment. The term “purified” does not imply that the sequence is the only nucleotide present, but that it is essentially free (about 90-95% pure) of non-nucleotide material naturally associated with it, and thus is distinguished from isolated chromosomes. As used herein, the terms “isolated” and “purified” in the context of a proteineous agent (e.g., a peptide, polypeptide, protein or antibody) refer to a proteineous agent which is substantially free of cellular material and in some embodiments, substantially free of heterologous proteineous agents (i.e. contaminating proteins) from the cell or tissue source from which it is derived, or substantially free of chemical precursors or other chemicals when chemically synthesized. The language “substantially free of cellular material” includes preparations of a proteineous agent in which the proteineous agent is separated from cellular components of the cells from which it is isolated and/or recombinantly and/or synthetically produced. Thus, a proteineous agent that is substantially free of cellular material includes preparations of a proteineous agent having less than about 30%, 20%, 10%, or 5% (by dry weight) of heterologous proteineous agent (e.g. protein, polypeptide, peptide, or antibody; also referred to as a “contaminating protein”). When the proteineous agent is recombinantly produced, it is also preferably substantially free of culture medium, i.e. culture medium represents less than about 20%, 10%, or 5% of the volume of the protein preparation. When the proteinaceous agent is produced by chemical synthesis, it is preferably substantially free of chemical precursors or other chemicals, i.e., it is separated from chemical precursors or other chemicals which are involved in the synthesis of the proteinaceous agent. Accordingly, such preparations of a proteinaceous agent have less than about 30%, 20%, 10%, 5% (by dry weight) of chemical precursors or compounds other than the proteinaceous agent of interest. Preferably, proteinaceous agents disclosed herein are isolated.

Still further, the determination of the level of expression of at least two of the biomarker protein/s of the present disclosure, specifically, at least two of C4BPB, KIF20B, VPS11, CRTAC1, TMEM67, GJA1 and ATP2B4 biomarker proteins, is performed by quantifying the proteins or peptides thereof in the sample, specifically, by the step of subjecting a biological sample of the subject, or any protein product obtained therefrom to a mass spectrometry assay.

It should be appreciated that in certain embodiments, the signature proteins, specifically, at least one, at least two, at least three, at least four, at least five, at least six or at least seven of the biomarker proteins of the invention or any protein-fragments thereof may be also detected and quantified without the need for detection molecule/s. Detection can be based on MS approaches using non-targeted or targeted methods such as selected reaction monitoring (SRM) or parallel reaction monitoring (PRM). These analyses can be performed with or without a reference heavy standard and provide quantitative measure of the peptide/protein amount. The heavy reference can be a synthetic peptide, or a chemically labeled peptide/protein or metabolically labeled proteins. In the absence of a standard, the MS signal can provide the measure of peptide abundance.

According to some embodiments, the method of the invention may use as a sample any one of a biological sample of body fluids, organ/s, cell/s or tissue/s or a blood sample.

As used herein, the term “sample” refers to cells, sub-cellular compartments thereof, tissue or organs. The tissue may be a whole tissue, or selected parts of a tissue. Tissue parts can be isolated by micro-dissection of a tissue, or by biopsy, or by enrichment of sub-cellular compartments. The term “sample” further refers to healthy as well as diseased or pathologically changed cells or tissues. Hence, the term further refers to a cell or a tissue associated with a disease, such a tumor, in particular carcinoma, ovarian cancer, and more specifically, High-grade ovarian carcinoma. A sample can be cells that are placed in or adapted to tissue culture. A sample can additionally be a cell or tissue from any mammalian species, specifically, humans. A tissue sample can be further a fractionated or preselected sample, if desired, preselected or fractionated to contain or be enriched for particular cell types.

In some specific and non-limiting embodiments, the sample of the method of the invention may be a body fluid sample. More specifically, such sample may be any body fluid such as blood, plasma, lymph, urine, saliva, serum, cerebrospinal fluid, seminal plasma, pancreatic juice, breast milk, uterine or lung lavage. More specifically, the sample may be uterine lavage sample. The term “lavage” as used herein refers to washing out an organ (such as the uterine), a body cavity, or a wound by flushing it with a fluid. The sample can be fractionated or preselected by a number of known fractionation or pre-selection techniques. A sample can also be any extract of the above. The term also encompasses protein fractions or alternatively, nucleic acid from cells or tissue. Thus, in some specific embodiments, the sample may be any one of a biological sample of organ/s, cell/s or tissue/s and a blood sample. In yet some other embodiments, the sample may be a primary tumor sample. In certain embodiments, the sample is obtained from a subject suffering from ovarian cancer.

Thus, in some further specific embodiments, the sample of the method of the invention may be microparticles/microvesicles prepared from said body fluid.

In some embodiments, the sample analyzed by the methods disclosed herein may be a body fluid sample.

In yet some further embodiments, the sample comprises microvesicles prepared from the body fluid.

Still further, in some specific embodiments, the body fluid is at least one of uterine lavage fluid (UtL) and plasma.

In certain embodiments, sample comprising microvesicles isolated from UtL, may be particularly useful in the methods disclosed herein.

“Microvesicles” (also known as ectosomes, microparticles or shedding microvesicles) are a type of extracellular vesicle (EV) that are formed by outward budding of the plasma membrane. The formation of microvesicles is dependent on the translocation of the phospholipid phosphatidylserine from the inner leaflet of the plasma membrane to the outer leaflet, which is carried out by aminophospholipid translocases.

The “utero-tubal lavage” or “UtL” refers herein to a minimally invasive technique to sample a liquid biopsy directly from the uterine cavity, which is continuous with the lumen of the fallopian tube fimbria.

It should be therefore appreciated that the invention provides in some embodiments thereof, a method that may further comprise at least part of the step of isolating microparticles/microvesicles from said body fluid sample, as well as at least part of the steps of isolating the sample. These procedures are described in more detailed herein below, as well as in the Experimental procedures section.

In some embodiments, the diagnostic methods disclosed herein are applicable for diagnosing ovarian cancer, specifically, high-grade ovarian carcinoma (HGOC), or OVCA.

Thus, in some embodiments, the diagnostic methods disclosed herein are applicable for early detection of OVCA in a subject.

An “early diagnosis” or “early detection” may be used interchangeably and provides diagnosis prior to appearance of clinical symptoms. Prior as used herein is meant days, weeks, months or even years before the appearance of such symptoms. More specifically, at least 1 week, at least 1 month, 2 months, 3 months, 4 months, 5 months, 6 months, 7 months, 8 months, 9 months, 10 months, 11 months, 12 months, or even few years before clinical symptoms appear.

It should be appreciated that the method of the invention may be suitable for any mammalian subject. By “patient” or “subject” it is meant any mammal that may be affected by the above-mentioned conditions, and to whom the treatment and diagnosis methods herein described is desired, including human, bovine, equine, canine, murine and feline subjects. Specifically, said subject is a human. Thus, in yet some further embodiments, the methods of the invention may be suitable for any mammalian female subject, specifically to any woman. In yet some further embodiments, the methods and kits of the invention may be suitable for any woman aged between 12 years to 90 or older.

In yet some further embodiments, the methods and kits of the invention may be suitable for early diagnosis of ovarian carcinoma in any woman over 30, 35, 40, 45, 50, 55, 60, 65, 70 years old, or even older.

In some specific and non-limiting embodiments, the method of the invention may be suitable for subjects that belong to a high-risk population. In some particular embodiments, such subject may be subject carrying at least one mutation in at least one gene associated or linked with an increased risk or high risk of ovarian cancer. In some particular embodiments, such genes may be any one of BRCA1, BRCA2, BARD1, BRIP1, CHEK2, MRE11A, MSH6, NBN, PALB2, RAD50, RAD51C, RAD5 1D, TP53, APC, ATM, DICER1, MUTYH, PALB2, RAD51 and MMR genes such as MLH1, MSH2, MSH6, and PMS2.

In some specific embodiments the methods, as well as the compositions and kits disclosed herein are applicable and are adapted for subjects that are classified as carriers of at least one mutation in at least one of BRCA1, and/or BRCA2 gene/s. more specifically, mutations in the genes BRCA1 or BRCA2 are the most prevalent among all the known susceptibility genes and correspond with up to 50% chance of developing the disease. The mutation in BRCA1 or BRCA2 DNA mismatch repair genes is present in 10% of ovarian cancer cases. Only one allele need be mutated to place a person at high risk, because the risky mutations are autosomal dominant. The gene can be inherited through either the maternal or paternal line but has variable penetrance. Though mutations in these genes are usually associated with increased risk of breast cancer, they also carry a substantial lifetime risk of ovarian cancer, a risk that peaks in a woman's 40s and 50s.

The lowest risk cited is 30% and the highest 60%. Mutations in BRCA1 have a lifetime risk of developing ovarian cancer of 15-45%. Mutations in BRCA2 are less risky than those with BRCA1, with a lifetime risk of 10% (lowest risk cited) to 40% (highest risk cited). On average, BRCA-associated cancers develop 15 years before their sporadic counterparts, because people who inherit the mutations on one copy of their gene only need one mutation to start the process of carcinogenesis, whereas people with two normal genes would need to acquire two mutations.

In some embodiments, the methods disclosed herein may enable monitoring of response to pre-operative treatment.

A further aspect of the present disclosure relates to a diagnostic method for detecting ovarian cancer in a subject. More specifically, in some embodiments, the method comprises the following steps: A first step (a), involves determining the expression level of at least two biomarker proteins in at least one biological sample of the diagnosed subject, to obtain an expression value for each of these at least two biomarker protein/s. More specifically, the at least two biomarker proteins may comprise, or in some embodiments, may be selected from at least one of the following options. In a first option (i) or model, the biomarker proteins may be at lest one of C4BPB and KIF20B. In the second option (ii) or model, the biomarker proteins may be at least one of, VPS11, CRTAC1 and TMEM67. In yet the third option (iii) or model, the biomarker proteins may be at least one of GJA1 and ATP2B4. It should be further noted that the option (iv), for any combination of at least two biomarker proteins of any one of the three models(i), (ii) and (iii), may be also used. In some optional embodiments, the disclosed signature, specifically or models 1, 2 3 and any combinations thereof as discussed herein, is specifically applicable for a subject determined as a carrier for at least one mutation in at least one gene associated with high risk for cancer, specifically, ovarian cancer. Specifically, carriers of a mutation in hereditary ovarian cancer genes also indicated herein as genetically predisposed population. A genetic predisposition, as used herein, refers to an individual's increased likelihood or susceptibility to developing a particular trait, condition, or disease due to genetic factors.

In some embodiments, increased likelihood of having a cancer, that may be more specifically, an ovarian cancer and/or breast cancer. It means that an individual carries certain genetic variations or mutations that make them more susceptible to a specific outcome, specifically, ovarian cancer. It should be understood that genetic predispositions can be inherited from one or both parents and can be caused by variations or mutations in specific genes or combinations of multiple genes. These genetic factors may interact with environmental factors to determine the likelihood of developing a certain trait or condition. It is important to note that having a genetic predisposition does not guarantee that an individual will develop the associated condition, specifically, ovarian cancer, but it increases their risk compared to individuals without the genetic predisposition.

As indicated above, the disclosed methods are specifically applicable for subjects that are carriers of at least one mutation and/or variation in at least one gene, that may be a pathogenic mutation, or a pathogenic-like mutation associated with ovarian cancer. In the context of disclosure, a “mutation” as used herein to describe any gene that carry at least one mutation associated with predisposition to cancer, specifically, ovarian cancer, refers to any change or alteration in the specific disclosed gene, specifically, any mutation in at least one of the genes disclosed by any one of SEQ ID NO: 29, 30, 31, 32, 33, 34, 35, 36, 37, 38 and/or 39. In some embodiments, mutation/s involve modifications to the DNA sequence, specifically, insertion, deletion, or substitution of nucleotides, rearrangement, translocation and the like, resulting in a variation in the genetic code. Still further, in some embodiments, mutations may encompass point mutations, specifically, any mutations involving a change in a single nucleotide base in the DNA sequence. Point mutations can be further classified into three categories: Silent Mutation: A nucleotide substitution that does not result in a change in the amino acid sequence of the protein encoded by the gene. Missense Mutation: A nucleotide substitution that leads to the incorporation of a different amino acid in the protein sequence, potentially altering its structure and function. And nonsense Mutation: A nucleotide substitution that generates a premature stop codon, resulting in the production of a truncated and usually nonfunctional protein.

Still further, in accordance with the present disclosure mutations further encompass insertion/s. As used herein an insertion mutation occurs when one or more nucleotides are added to the DNA sequence. This can lead to a shift in the reading frame during protein synthesis, potentially altering the entire protein sequence downstream of the mutation.

In yet some further embodiments, mutations may refer to deletions. A deletion mutation involves the removal of one or more nucleotides from the DNA sequence. Like insertions, deletions can cause a shift in the reading frame, resulting in a different protein sequence. Still further, in some embodiments, the term “mutation/s” as used herein further encompasses duplication/s. Duplication mutations involve the replication of a segment of DNA, leading to the presence of multiple copies of a particular sequence within the genome. This can have various effects, including gene dosage imbalance and the emergence of new functional elements.

In yet some further embodiments, mutation/s also include herein inversion/s. An inversion mutation occurs when a segment of DNA is reversed or flipped within the chromosome. This can disrupt gene expression patterns and alter the function of affected genes.

Still further, translocations are also encompassed by the term mutation/s as used in the present disclosure. Translocation mutations involve the transfer of a segment of DNA from one chromosome to another, potentially leading to rearrangements of genetic material. Translocations can have significant consequences on gene regulation and can be associated with certain genetic disorders.

In some embodiments, the subject is a subject that carry at least one mutation in at least one of BRCA1, BRCA2, BRIP1, PALB2, RAD51, BARD1, CHEK2 and MMR genes such as MLH1, MSH2, MSH6, and PMS2. Specifically, as denoted by any one of SEQ ID NO: 29-39. It should be understood that in some embodiments any mutation in each one of he specified genes may be applicable in the present disclosure, for example, any of the mutations disclosed in the CLINVAR database.

Still further, in some embodiments the mutations in the carriers may be either germline mutation or somatic mutations. In some embodiments, the mutations may be germline mutation/s and/or variations. A germline mutation, or germinal mutation, is any detectable variation within germ cells (cells that, when fully developed, become sperm and ova). Mutations in these cells are the only mutations that can be passed on to offspring, when either a mutated sperm or oocyte come together to form a zygote.

The next step of the diagnostic methods disclosed herein (b), involves determining if the expression value obtained in step (a) for each of the at least two biomarker protein/s is altered (for example, is positive or negative) with respect to a predetermined standard expression value or to an expression value of the biomarker protein/s in at least one control sample.

More specifically, a positive expression value, or in other words, elevated expression, high expression or upregulation of at least one of (i) C4BPB; (ii) at least one of VPS11, CRTAC1 and TMEM67; (iii) at least one of GJA1 and ATP2B4 and/or a negative expression value, or down regulation of KIF20B in the sample as compared with a predetermined standard expression value or to an expression value of the biomarker protein/s in at least one control sample, indicates that the subject has ovarian cancer, thereby diagnosing the subject.

The next step (c) of the disclosed methods involves administering to a subject classified as suffering of, or having ovarian cancer, an effective amount of at least one anti-cancer agent, and/or subjecting said subject to an anti cancer treatment regimen.

It should be appreciated that various embodiments of the disclosed methods are as defined for the diagnostic method of the present disclosure as indicated herein above.

A further aspect of the present disclosure relates to a method for treating, preventing and/or ameliorating ovarian cancer in a subject in need thereof. The method comprises the following steps:

A first step (a), involves detecting ovarian cancer in said subject by the following steps: (I) determining the expression level of at least two biomarker proteins in at least one biological sample of the diagnosed subject, to obtain an expression value for each of these at least two biomarker protein/s. More specifically, the at least two biomarker proteins may comprise, or in some embodiments, may be selected from at least one of the following options. In a first option (i) or model, the biomarker proteins may be at lest one of C4BPB and KIF20B. In the second option (ii) or model, the biomarker proteins may be at least one of, VPS11, CRTAC1 and TMEM67. In yet the third option (iii) or model, the biomarker proteins may be at least one of GJA1 and ATP2B4. It should be further noted that the option (iv), for any combination of at least two biomarker proteins of any one of the three models(i), (ii) and (iii), may be also used. In some optional embodiments, the disclosed signature, specifically or models 1, 2 3 and any combinations thereof as discussed herein, is specifically applicable for a subject determined as a carrier for at least one mutation in at least one gene associated with high risk for cancer, specifically, ovarian cancer. Specifically, carriers of a mutation in hereditary ovarian cancer genes also indicated herein as genetically predisposed population.

Next (II), the diagnostic step disclosed herein involves determining if the expression value obtained in step (I) for each of the at least two biomarker protein/s is altered (for example, is positive or negative) with respect to a predetermined standard expression value or to an expression value of the biomarker protein/s in at least one control sample.

More specifically, a positive expression value, or in other words, elevated expression, high expression or upregulation of at least one of (i) C4BPB; (ii) at least one of VPS11, CRTAC1 and TMEM67; (iii) at least one of GJA1 and ATP2B4 and/or a negative expression value, or down regulation of KIF20B in the sample as compared with a predetermined standard expression value or to an expression value of the biomarker protein/s in at least one control sample, indicates that the subject has ovarian cancer, thereby diagnosing the subject.

The next step (b) of the disclosed therapeutic methods involves administering to a subject classified as suffering of, or having ovarian cancer, an effective amount of at least one anti-cancer agent, and/or subjecting said subject to an anti cancer treatment regimen.

It should be appreciated that various embodiments of the disclosed methods are as defined for the diagnostic method of the present disclosure as indicated herein above.

In yet a further aspect, the present disclosure provides an effective amount of at least one anti-cancer agent for use in a method for treating, preventing and/or ameliorating ovarian cancer in a subject in need thereof. The method comprises the following steps:

A first step (a), involves detecting ovarian cancer in said subject by the following steps: (I) determining the expression level of at least two biomarker proteins in at least one biological sample of the diagnosed subject, to obtain an expression value for each of these at least two biomarker protein/s. More specifically, the at least two biomarker proteins may comprise, or in some embodiments, may be selected from at least one of the following options. In a first option (i) or model, the biomarker proteins may be at lest one of C4BPB and KIF20B. In the second option (ii) or model, the biomarker proteins may be at least one of, VPS11, CRTAC1 and TMEM67. In yet the third option (iii) or model, the biomarker proteins may be at least one of GJA1 and ATP2B4. It should be further noted that the option (iv), for any combination of at least two biomarker proteins of any one of the three models(i), (ii) and (iii), may be also used. In some optional embodiments, the disclosed signature, specifically or models 1, 2 3 and any combinations thereof as discussed herein, is specifically applicable for a subject determined as a carrier for at least one mutation in at least one gene associated with high risk for cancer, specifically, ovarian cancer. Specifically, carriers of a mutation in hereditary ovarian cancer genes also indicated herein as genetically predisposed population.

Next (II), the diagnostic step disclosed herein involves determining if the expression value obtained in step (I) for each of the at least two biomarker protein/s is altered (for example, is positive or negative) with respect to a predetermined standard expression value or to an expression value of the biomarker protein/s in at least one control sample.

More specifically, a positive expression value, or in other words, elevated expression, high expression or upregulation of at least one of (i) C4BPB; (ii) at least one of VPS11, CRTAC1 and TMEM67; (iii) at least one of GJA1 and ATP2B4 and/or a negative expression value, or down regulation of KIF20B in the sample as compared with a predetermined standard expression value or to an expression value of the biomarker protein/s in at least one control sample, indicates that the subject has ovarian cancer, thereby diagnosing the subject.

The next step (b) of the disclosed therapeutic use involves administering to a subject classified as suffering of, or having ovarian cancer, an effective amount of the at least one anti-cancer agent, and/or subjecting said subject to an anti cancer treatment regimen.

A further aspect of the present disclosure relates to a diagnostic composition comprising at least two detecting molecules or any combination or mixture of plurality of detecting molecules specific for determining the level of expression of at least two biomarker protein/s of the present disclosure. In some embodiments, the at least two biomarker protein/s may comprise, or in some embodiments, may be selected from at least one of: (i) at least one of C4BPB and KIF20B; (ii) at least one of VPS11, CRTAC1 and TMEM67; (iii) at least one of GJA1 and ATP2B4; and (iv) any combination of at least two biomarker proteins of any one of (i), (ii) and (iii) protein/s.

In some optional embodiments, that may be either in addition to or as an alternative, the composition may comprise detecting molecules specific for at least two of CTSD, SPTBN2, ARFIP1, MIA3, CD109, IRGQ and GOLPH3 protein/s or any combination thereof. In some embodiments, each of the detecting molecules is specific for one of the biomarker protein/s.

In some embodiments, the diagnostic composition of the present disclosure may comprise at least four detecting molecules or any combination or mixture of plurality of detecting molecules specific for determining the level of expression of at least four biomarker protein/s of the present disclosure. In some embodiments, the at least four biomarker protein/s are selected from at least one of: (i) at least one of C4BPB and KIF20B; (ii) at least one of VPS11, CRTAC1 and TMEM67; (iii) at least one of GJA1 and ATP2B4; and (iv) any combination of at least two biomarker proteins of any one of (i), (ii) and (iii) protein/s.

In some optional embodiments, that may be either in addition to or as an alternative, the composition may comprise detecting molecules specific for at least four of CTSD, SPTBN2, ARFIP1, MIA3, CD109, IRGQ and GOLPH3 protein/s or any combination thereof. In some embodiments, each of the detecting molecules is specific for one of the biomarker protein/s.

In some specific embodiments, the compositions disclosed herein may comprise detecting molecules or any combination or mixture of plurality of detecting molecules specific for determining the level of expression of C4BPB, KIF20B, VPS11, CRTAC1, TMEM67, GJA1 and ATP2B4 protein/s or any combination thereof.

Still further, in some optional alternative and/or additional embodiments, the disclosed compositions may comprise detecting molecules or any combination or mixture of plurality of detecting molecules specific for determining the level of expression of CTSD, SPTBN2, ARFIP1, MIA3, CD109, IRGQ and GOLPH3 protein/s or any combination thereof. It should be noted that each of the detecting molecules is specific for one of the biomarker proteins.

In some embodiments, the detecting molecules of the compositions of the present disclosure are selected from amino acid detecting molecules and nucleic acid detecting molecules, or any combinations thereof.

Still further, in some embodiments, the composition disclosed herein may comprise amino acid detecting molecules. In more specific embodiments, such compositions may comprise at least one of the following options. In option (a), at least two labeled or tagged protein/s or any fragment/s, peptide/s or mixture/s thereof, of at least one of: (i) at least one of C4BPB and KIF20B; (ii) at least one of VPS11, CRTAC1 and TMEM67; (iii) at least one of GJA1 and ATP2B4; and (iv) any combination of at least two biomarker proteins of any one of (i), (ii) and (iii), protein/s or any fragment/s, peptide/s or mixture/s thereof.

In some optional embodiments, that may be either in addition to or as an alternative, at least two labeled or tagged protein/s or any fragment/s, peptide/s or mixture/s thereof of at least two of CTSD, SPTBN2, ARFIP1, MIA3, CD109, IRGQ and GOLPH3 protein/s or any fragment/s, peptide/s or mixture/s thereof.

In yet another alternative or additional embodiments the detecting molecules of the compositions may comprise (b), at least one antibody specific for the at least two of the biomarker protein/s. Still further, in option (c), the composition may comprise at least one peptide aptamer/s specific for at least one of said at least two biomarker protein/s. It should be appreciated however, that in some embodiments (d), the compositions of the present disclosure may comprise any combination of detecting molecules as disclosed in (a), (b) and (c).

In yet some further embodiments, the compositions disclosed herein may comprise nucleic acid detecting molecules. In some specific embodiments, such nucleic acid detecting molecules may comprise at least one of: (a), at least one nucleic acid aptamer/s, each aptamer is specific for at least one of the at least two biomarker proteins; and/or (b), at least one oligonucleotide/s, each oligonucleotide specifically hybridizes to a nucleic acid sequence encoding at least one of the at least two biomarker protein/s.

In some embodiments, the detecting molecules are attached to a solid support.

It should be noted that each of the detecting molecules is specific for one of the biomarker proteins. It should be appreciated that in certain embodiments, the composition of the invention may be at least one of diagnostic composition. In certain embodiments, the detecting molecules comprised within the composition of the invention may be attached to a solid support. Definitions of solid support that may be used as part of the diagnostic composition of the invention are described in more detail herein after, in connection with the kit of the invention. It should be appreciated that in some specific and non-limiting embodiments, the detecting molecules of the composition of the invention may be provided in a suitable medium or a buffer. In some alternative embodiments, the detecting molecules of the invention may be provided in a dried form.

It should be appreciated that the invention encompasses compositions comprising detecting molecules specific for any combination of any of the marker protein used by the invention.

Still further, in some embodiments, the detecting molecules are provided in a mixture.

In some embodiment, the composition of the invention may comprise at least one detecting molecule or any combination or mixture of plurality of detecting molecules specific for determining the level of expression of at least two of the biomarkers of the present disclosure.

In some embodiments, the composition of the invention may comprise detecting molecules specific for at least one further additional biomarker. In more specific embodiments, the compositions of the invention may comprise also detecting molecule/s specific for at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 or more, specifically, 110, 120, 130, 140, 150, 160, 170, 180, 190, 200, 250, 300, 350, 384, 400, 450, 460, 470, 480, 490, 493 at the most additional biomarkers and/or control proteins, such that a total number of 500 at the most, the biomarker of the present disclosure and additional biomarker proteins and/or control proteins or reference proteins.

According to some embodiments, the detecting molecules suitable for the composition of the invention may be selected from amino acid detecting molecules and nucleic acid detecting molecules.

It should be appreciated that in more specific embodiments, the compositions of the invention may comprise detecting molecules specific for at least one additional biomarker protein, specifically, at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 or more, specifically, 110, 120, 130, 140, 150, 160, 170, 180, 190, 200, 250, 300, 350, 400, 450, 460, 470, 480, 490, 493 at the most additional biomarkers, and/or control proteins, such that a total number of 500 at the most, the biomarker of the present disclosure and additional biomarker proteins and/or control proteins or reference proteins.

As noted above, it should be appreciated that any of the compositions of the invention may be used for early diagnosis of ovarian carcinoma, specifically, OVCA, more specifically, HGOC.

The detecting molecules of the invention were described in detailed in connection with the methods of the invention. It should be appreciated that all embodiments for detecting molecules mentioned therein are also applicable for the compositions and kits of the invention.

A further aspect of the present disclosure relates to a kit comprising:
(a), at least two detecting molecules specific for determining the level of expression of at least two biomarker protein/s in a biological sample. In some embodiments the at least two biomarker protein/s may comprise, or in some embodiments, are selected from at least one of: (i) at least one of C4BPB and KIF20B; (ii) at least one of VPS11, CRTAC1 and TMEM67; (iii) at least one of GJA1 and ATP2B4; and (iv) any combination of at least two biomarker proteins of any one of (i), (ii) and (iii) protein/s or any combination thereof.

In some optional alternative or additional embodiments, where model 4 as discussed herein is applicable, the kits of the present disclosure may comprise detecting molecules specific for determining the level of expression of at least two of CTSD, SPTBN2, ARFIP1, MIA3, CD109, IRGQ and GOLPH3 protein/s or any combination thereof.

It should be noted that each of the detecting molecule/s is specific for one of the biomarker proteins. In some embodiments, the kits disclosed herein may optionally further comprise at least one of:
(b), pre-determined calibration curve/s or predetermined standard/s providing standard expression values of the at least two biomarker/s; and/or
(c), at least one control sample.

In yet some further embodiments, the disclosed kits may comprise:
(a), at least two detecting molecules specific for determining the level of expression of at least four biomarker protein/s in a biological sample. In some embodiments the at least four biomarker protein/s may be selected from at least one of: (i) at least one of C4BPB and KIF20B; (ii) at least one of VPS11, CRTAC1 and TMEM67; (iii) at least one of GJA1 and ATP2B4; and (iv) any combination of at least four biomarker proteins of any one of (i), (ii) and (iii) protein/s or any combination thereof.

It should be noted that each of the detecting molecule/s is specific for one of the biomarker proteins. In some embodiments, the kits disclosed herein may optionally further comprise at least one of:
(b), pre-determined calibration curve/s or predetermined standard/s providing standard expression values of the at least two biomarker/s; and/or
(c), at least one control sample.

In some specific embodiments, the kit of the present disclosure may comprise the following components:
(a), detecting molecules specific for determining the level of expression of C4BPB, KIF20B, VPS11, CRTAC1, TMEM67, GJA1 and ATP2B4 protein/s or any combination thereof. It should be noted that each of the detecting molecule/s is specific for one of the biomarker proteins. In some embodiments, the kits disclosed herein may optionally further comprises at least one of:
(b), pre-determined calibration curve/s or predetermined standard/s providing standard expression values of said biomarker protein/s; and (c), at least one control sample.

In some embodiments, the detecting molecules of the kits disclosed herein are selected from amino acid detecting molecule/s, nucleic acid detecting molecule/s, and any combinations thereof.

In yet some further embodiments, the amino acid detecting molecules comprise at least one of: In some options (a), at least two labeled or tagged protein/s or any fragment/s, peptide/s or mixture/s thereof, of at least one of: (i) at least one of C4BPB and KIF20B; (ii) at least one of VPS11, CRTAC1 and TMEM67; (iii) at least one of GJA1 and ATP2B4; and (iv) any combination of at least two biomarker proteins of any one of (i), (ii) and (iii) protein/s or any fragment/s, peptide/s or mixture/s thereof.

Still further in some optional alternative and/or additional embodiments the amino acid detecting molecules of the disclosed kits may be based on model 4 and comprise at least two labeled or tagged protein/s or any fragment/s, peptide/s or mixture/s thereof, of at least two of CTSD, SPTBN2, ARFIP1, MIA3, CD109, IRGQ and GOLPH3 protein/s or any fragment/s, peptide/s or mixture/s thereof.

In some further optional embodiments (b), the kits may comprise at least one antibody specific for at least one of the at least two biomarker proteins. In yet some further embodiments (c), at least one peptide aptamer/s specific for one of the at least two biomarker protein/s. It should be noted that the kits of the present disclosure may comprise (d), detecting molecules of any combination of (a), (b) and (c).

In some embodiments, the kits of the present disclosure may comprise at least one nucleic acid detecting molecule. Such detecting molecules may comprise at least one of: (a), at least one nucleic acid aptamer/s specific for at least one of the at least two biomarker proteins. Alternatively of additionally, the kits disclosed herein may comprise (b), at least one oligonucleotide, each oligonucleotide specifically hybridizes to a nucleic acid sequence encoding at least one of the at least two biomarker protein/s.

Still further, in some embodiments, the detecting molecule/s of the kits disclosed herein are attached to a solid support.

Still further, in some embodiments the detecting molecule/s of the kits disclosed herein may be provided in a mixture.

In some embodiments, the kits of the invention may further comprise instructions for use.

In yet some further embodiments, the instructions comprise at least one of: (a), instructions for carrying out the detection and quantification of the expression of the at least two biomarker proteins of at least one of: (i) at least one of C4BPB and KIF20B; (ii) at least one of VPS11, CRTAC1 and TMEM67; (iii) at least one of GJA1 and ATP2B4; and (iv) any combination of at least two biomarker proteins of any one of (i), (ii) and (iii) biomarker protein/s,

Still further in some optional alternative and/or additional embodiments the amino acid detecting molecules of the disclosed kits may be based on model 4 and comprise instructions that relate to at least two of CTSD, SPTBN2, ARFIP1, MIA3, CD109, IRGQ and GOLPH3 biomarker protein/s. In some optional embodiments, the kits may comprise at least one control reference protein. Still further the kits may comprise in some embodiments (b), instructions for determining if the expression values of at least two of the biomarker protein/s is positive or negative with respect to a corresponding predetermined standard expression value or with expression value of at least one of the at least two biomarker protein/s in the at least one control sample.

In yet some further embodiments, the kits may further comprise at least one reagent for conducting a mass spectrometry assay.

In yet some further embodiments, the kits may further comprise at least one reagent for conducting an immunological assay.

In certain embodiments, the kits disclosed herein may further comprise at least one device or means for obtaining a body fluid sample and for isolating microvesicles from said body fluid sample.

Still further, in some embodiments, the kits disclosed herein may be for use in a method for detecting ovarian cancer in a subject. In more specific embodiments, the kits disclosed herein may be used in any of the diagnostic methods disclosed by the present invention.

Still further, the kits of the present disclosure may be adapted for detecting ovarian cancer in a subject. Thus, in some embodiments, the kits disclosed herein are diagnostic kits.

In some specific embodiments, the diagnostic kits disclosed herein ovarian cancer is specifically applicable for diagnosis of High-grade ovarian carcinoma (HGOC).

In yet some further embodiments, the kits disclosed herein may be used in diagnostic methods of early detection of High-grade ovarian carcinoma.

Still further, in some embodiments a body fluid sample may be used by the kits of the present disclosure.

In more specific embodiments, the sample that may be analyzed by the kits of the invention may be microvesicles prepared from the body fluid.

In more specific embodiments, the body fluid is at least one of uterine lavage fluid (UtL) and plasma.

In yet some further specific embodiments, a sample comprising microvesicles isolated from UtL, may be useful in the kits of the invention.

Still further, the inventors consider the kit of the invention in compartmental form. It should be therefore noted that in certain embodiments the detecting molecules used for detecting the expression levels of the biomarker proteins may be provided in a kit attached to an array. As defined herein, a “detecting molecule array” refers to a plurality of detection molecules that may be nucleic acids based or protein based detecting molecules, optionally attached to a support where each of the detecting molecules is attached to a support in a unique pre-selected and defined region.

For example, an array may contain different detecting molecules, such as specific antibodies, labeled or tagged proteins, peptides, aptamers, probes and/or primers or any combinations thereof. As indicated herein before, in case a combined detection of the biomarker proteins expression level, the different detecting molecules for each target may be spatially arranged in a predetermined and separated location in an array. For example, an array may be a plurality of vessels (test tubes), plates, micro-wells in a micro-plate, each containing different detecting molecules, specifically, aptamers, primers and antibodies, specific for each marker protein used by the invention. An array may also be any solid support holding in distinct regions (dots, lines, columns) different and known, predetermined detecting molecules.

As used herein, “solid support” is defined as any surface to which molecules may be attached through either covalent or non-covalent bonds. Thus, useful solid supports include solid and semi-solid matrixes, such as aero gels and hydro gels, resins, beads, biochips (including thin film coated biochips), micro fluidic chip, a silicon chip, multi-well plates (also referred to as microtiter plates or microplates), membranes, filters, conducting and no conducting metals, glass (including microscope slides) and magnetic supports. More specific examples of useful solid supports include silica gels, polymeric membranes, particles, derivative plastic films, glass beads, cotton, plastic beads, alumina gels, polysaccharides such as Sepharose, nylon, latex bead, magnetic bead, paramagnetic bead, super paramagnetic bead, starch and the like. This also includes, but is not limited to, microsphere particles such as Lumavidin™ or LS-beads, magnetic beads, charged paper, Langmuir-Blodgett films, functionalized glass, germanium, silicon, PTFE, polystyrene, gallium arsenide, gold, and silver. Any other material known in the art that is capable of having functional groups such as amino, carboxyl, thiol or hydroxyl incorporated on its surface, is also contemplated. This includes surfaces with any topology, including, but not limited to, spherical surfaces and grooved surfaces.

It should be further appreciated that any of the reagents, substances or ingredients included in any of the methods and kits of the invention may be provided as reagents embedded, linked, connected, attached, placed or fused to any of the solid support materials described above.

In certain embodiments, the detecting molecule/s used in the kit of the invention may be provided in a mixture. In some alternative embodiments, the detecting molecules may be provided as molecules that are not attached to any solid support. In some embodiments, the non-attached detecting molecules may be provided in separate containers, wells, tube vessels and the like. In some alternative embodiments, the attached or non-attached detecting molecules may be provided in a mixture that contains at least two detecting molecules specific for at least two biomarker protein/s of the invention.

It should be understood that any of the detecting molecules described by the invention are also applicable for this aspect.

It should be appreciated that the components in the kit may depend on the method of detection and are not limited to any method. In some embodiments, the kit of the invention may further comprise at least one reagent for conducting a mass spectrometry assay. Such reagents may include trypsin, buffers, filters and the like, for peptide purification.

In some other embodiments, the kit of the invention further comprising at least one reagent for conducting an immunological assay selected from protein microarray analysis, ELISA, RIA, slot blot, dot blot, FACS, western blot, immunohistochemical assay, immunofluorescent assay and a radio-imaging assay.

In further embodiments, the kit of the invention may further comprise at least one device, means or any reagent for obtaining a body fluid sample, specifically UtL and for isolating microparticles/microvesicles from said body fluid sample.

In some other embodiments, the kit of the invention may be for use in a method for detecting ovarian cancer in a subject.

In certain embodiments, the kit of the invention may be suitable for use in a method for detecting High-grade ovarian carcinoma.

In yet another embodiment, the kit of the invention may be suitable for use in a method of early detection of High-grade ovarian carcinoma.

In accordance with some other embodiments, the sample to be used is any one of a body-fluid a liquid biopsy and a blood sample. Still further, according to certain embodiments, the kits of the invention may use any appropriate biological sample. The term “biological sample” in the present specification and claims is meant to include samples obtained from a mammalian subject.

In some embodiments, the biological sample may be a bodily fluid, a tissue, a tissue biopsy, a skin swab, an isolated cell population or a cell preparation.

In some specific embodiments, the population of cells comprises cancer cells. In another embodiment the population of cells is an in vitro cultured cell population.

In some embodiments, the biological sample may be a bodily fluid selected from the group consisting of utero-tubal wash, blood, serum, plasma, urine, cerebrospinal fluid, amniotic fluid, tear fluid, nasal wash, mucus, saliva, sputum, broncheoalveolar fluid, throat wash, vaginal fluid. In a specific embodiment, the biological sample is uterine lavage sample.

In certain embodiments, the sample used in the kit of the invention may be a body fluid sample. The kits of the invention may therefore further comprise any suitable means or device for obtaining said sample.

In yet another embodiment, the sample used for the kit of the invention may be microvesicles prepared from said body fluid.

In certain embodiments, the body fluid suitable for the kit of the invention may be at least one of UtL and plasma.

In some embodiments, the sample suitable for the kit of the invention may comprise microvesicles isolated from UtL.

One of the challenges associated with cancer and specifically ovarian cancer treatment originates from non-efficient treatments or resistance to treatment. Thus, the present invention further provides the use of at least one of the biomarker proteins as markers for evaluating response of patients treated with a certain therapeutic agent or monitoring the efficacy of treatment with a certain therapeutic agent. In some embodiments, the method of the invention may be particularly suitable for monitoring and early diagnosis of response of the diagnosed disorder in the subject.

In yet some further aspect, the invention may further provide a method for monitoring the efficacy of a treatment with a therapeutic agent and the disease progression.

It should be understood that the prognostic and monitoring methods offered by the invention may be applicable for patients that are treated with any therapeutic compound.

In more specific embodiments, such patient has not been subjected to reducing bilateral salpingo-oophorectomy (RRBSO), or any surgical intervention.

The therapy according with the present invention may be any therapy applicable to cancer and specifically to ovarian cancer. In some embodiments, for subjects classified as patients suffering from ovarian cancer by the methods of the invention, an endocrine therapy or any combination thereof with a biological therapy may be offered. Endocrine therapy refers to a treatment that adds, blocks, or removes hormones. In the context of the present disclosure, endocrine therapy is provided to slow or stop the growth of ovarian cancers. In this connection, synthetic hormones or other drugs may be given to block the body's natural hormones. In yet some further embodiments, therapy based on aromatase inhibitors may be offered. Other therapeutic options may also include biological therapy (antibodies and the like) and cryotherapy. In yet some other embodiments, where the subject is classified as an ovarian cancer suffering patient, chemotherapy, radiotherapy or any combinations thereof may be offered.

As detailed herein, the method of the invention may be also applicable for evaluating or monitoring the responsiveness of a patient, specifically a patient that was not subjected to RRBSO, to treatment with any therapeutic agent or regimen. Accordingly, the patient may be evaluated in at least one time point after initiation of treatment in order to assess if the treatment protocol is efficient and appropriate. Determination can be carried out at an early time points such that a decision may be made regarding continuation of the treatment or alternatively readjusting the treatment protocol.

In yet some other embodiments, the invention further provides a method for assessing responsiveness of a mammalian subject, specifically, a human subject to treatment with a specific therapeutic agent or evaluating and/or monitoring the efficacy of treatment on a subject. This method is based on determining the expression values of the biomarkers of the invention before and any time after initiation of treatment and calculating the ratio of the change in said values as a result of the treatment.

As indicated above, in accordance with some embodiments of the invention, in order to assess the patient condition, or monitor the disease progression, as well as responsiveness to a certain treatment, at least two “temporally-separated” test samples must be collected from the examined patient and compared thereafter in order to obtain the rate of change in the expression value of at least one of the biomarker proteins between said samples. In practice, to detect a change in at least one of these parameters between said samples, at least two “temporally-separated” test samples and preferably more must be collected from the patient.

The expression value is then determined using the method of the invention, applied for each sample. As detailed above, the rate of change in parameters is calculated by determining the ratio between at least two values of expression obtained from the same patient in different time-points or time intervals.

This period of time, also referred to as “time interval”, or the difference between time points (wherein each time point is the time when a specific sample was collected) may be any period deemed appropriate by medical staff and modified as needed according to the specific requirements of the patient and the clinical state he or she may be in. For example, this interval may be at least one day, at least three days, at least three days, at least one week, at least two weeks, at least three weeks, at least one month, at least two months, at least three months, at least four months, at least five months, at least one year, or even more.

In some embodiments, one of the time points may correspond to a period in which a patient is experiencing a remission of the disease.

When calculating the rate of change, one may use any two samples collected at different time points from the patient. To ensure more reliable results and reduce statistical deviations to a minimum, averaging the calculated rates of several sample pairs is preferable. A calculated or average value of a negative rate of change of the expression value of at least one of said biomarker protein/s indicates that said subject exhibits a beneficial response to said treatment; thereby monitoring the efficacy of a treatment with a therapeutic agent and the disease progression. It should be noted that in certain embodiments, where normalization step is being performed, the values referred to above, are normalized values.

As indicated above, the invention provides diagnostic and prognostic methods. “Prognosis” is defined as a forecast of the future course of a disease or disorder, based on medical knowledge. This highlights the major advantage of the invention, namely, the ability to predict progression of the disease, based on the expression value of at least one of the biomarker proteins. More specifically, the ability to determine at early stage that the subject is suffering from ovarian cancer, or in some specific embodiments, OVCA, and specifically, HGOC. This ability facilitates the selection of appropriate treatment regimen/s that may minimize side effects from unnecessary treatment, particularly, surgical intervention, individually to each patient, as part of personalized medicine.

Still further, as indicated above, in order to execute the prognostic method of the invention, at least two different samples must be obtained from the subject in order to calculate the rate of change in the expression as detailed above. By obtaining at least two and preferably more biological samples from a subject and analyzing them according to the method of the invention, the prognostic method may be effective for predicting, monitoring and early diagnosing molecular alterations indicating response to treatment in said patient.

All scientific and technical terms used herein have meanings commonly used in the art unless otherwise specified. The definitions provided herein are to facilitate understanding of certain terms used frequently herein and are not meant to limit the scope of the present disclosure.

The term “about” as used herein indicates values that may deviate up to 1%, more specifically 5%, more specifically 10%, more specifically 15%, and in some cases up to 20% higher or lower than the value referred to, the deviation range including integer values, and, if applicable, non-integer values as well, constituting a continuous range.

Thus, as used herein the term “about” refers to ±10%.

The terms “comprises”, “comprising”, “includes”, “including”, “having” and their conjugates mean “including but not limited to”. This term encompasses the terms “consisting of” and “consisting essentially of”. The phrase “consisting essentially of” means that the composition or method may include additional ingredients and/or steps, and/or parts, but only if the additional ingredients and/or steps do not materially alter the basic and novel characteristics of the claimed composition or method. Throughout this specification and the Examples and claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

It should be noted that various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible sub ranges as well as individual numerical values within that range. For example, description of a range such as from 1 to 6 should be considered to have specifically disclosed sub ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range. Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals there between.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub combination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

Disclosed and described, it is to be understood that this invention is not limited to the particular examples, methods steps, and compositions disclosed herein as such methods steps and compositions may vary somewhat. It is also to be understood that the terminology used herein is used for the purpose of describing particular embodiments only and not intended to be limiting since the scope of the present invention will be limited only by the appended claims and equivalents thereof.

It must be noted that, as used in this specification and the appended claims, the singular forms “a”, “an” and “the” include plural referents unless the content clearly dictates otherwise.

### EXAMPLES

### Experimental Procedures

### Cohort Design and Assembly

All uterine lavage samples were collected in accordance with approvals of the institutional ethics review boards at Chaim Sheba Medical Center, Rabin Medical Center, Shaare Zedek and Meir Medical Center, Israel (ClinicalTrials.gov identifier: NCT03150121). Informed consent was obtained from each participant. Recruited patients underwent gynecological surgical procedures under general anesthesia, including hysteroscopy, laparoscopic surgery or laparotomy. Eligible indications included high grade ovarian cancer (primary or interval debulking), suspicious ovarian mass, risk reduction, or various other benign gynecological disorders. Patients with other gynecological malignancies were excluded, as well as patients with non-high grade serous ovarian tumors. Pregnant women, women who were trying to conceive or menstruating at the time of clinic visit or patients with a legal guardian were also excluded. Pathology reports and medical records were available to the inventors in all cases.

The following groups of participants were recruited:

(1) Clinically healthy BRCA1/2 mutation carriers who have not yet undergone risk reducing salpingo-oophorectomy (RRSO): utero-tubal lavage (UtL) is performed either in a clinic setting or in an operating room immediately before their RRSO surgery; This group is defined herein as high-risk control.

(2) HGOC (high grade ovarian cancer) patients, regardless of their BRCA status: UtL performed immediately before debulking surgery;

(3) Average-risk controls: UtL performed in a clinic setting or in an operating room. This group is defined herein as healthy controls.

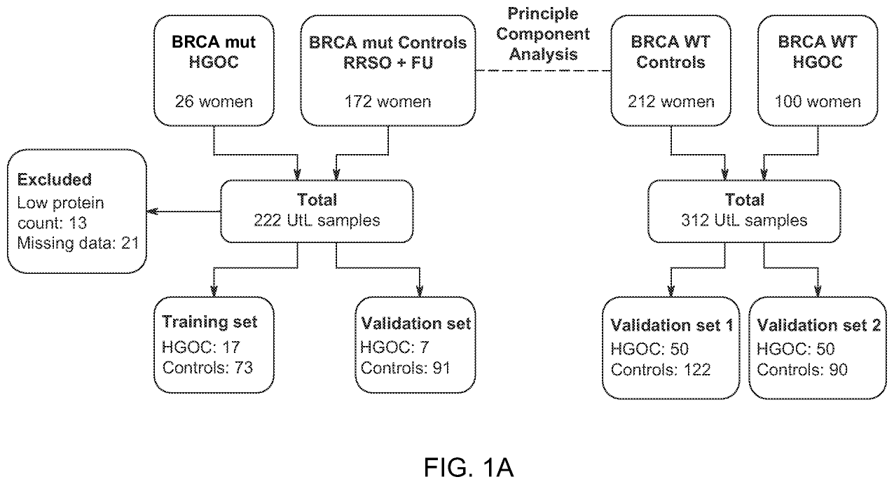

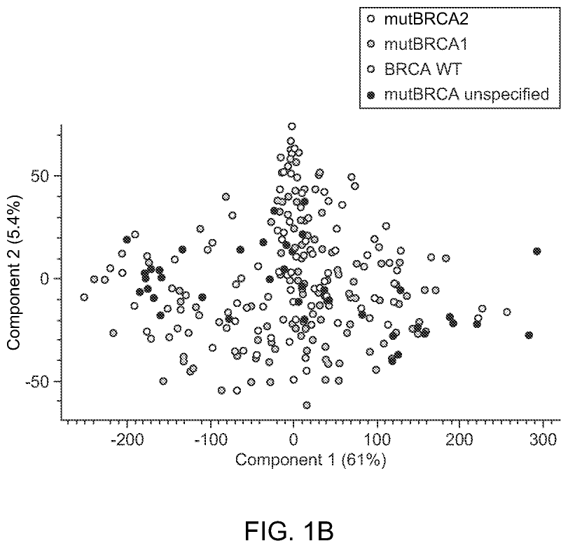

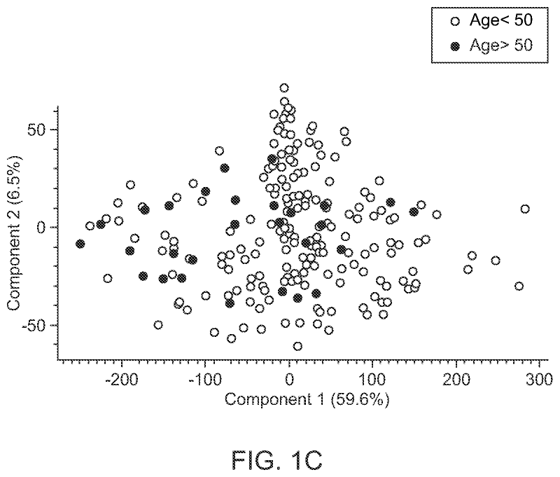

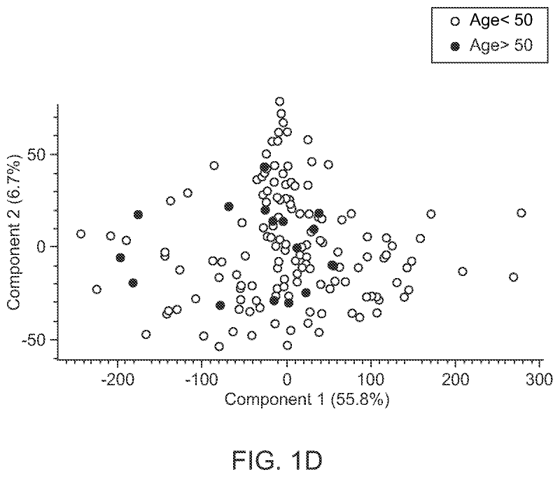

To develop the diagnostic classifier, the inventors analyzed 222 UtL samples of BRCA carriers, in several batches according to their dates. After exclusion of samples with low protein count or missing data, the first batches, with a total of 90 samples were included in the training set (17 HGOC patients and 73 controls), and all subsequent samples were regarded as a validation set (n=98, 7 HGOC and 91 controls), and analyzed independently in a blinded manner. The sample distribution is illustrated in FIG. 1A. Additional information can be found in Table 1. Additional data and information with respect to the examined patients is as disclosed in the corresponding publication by the inventors (Proteomic signature for detection of high-grade ovarian cancer in germline BRCA mutation carriers, Int. J. Cancer (2023) 15; 152(4):781-793, see Tables 1S, 2S, 3S therein, incorporated herein by reference).

Two additional validation sets, included 172 and 140 samples of UtL from women with BRCA-WT or unknown BRCA status, were used to test the accuracy of the classifier for use in the general population. Information about the first cohort has been previously published by Barnabas et al. [14].

Sample processing was performed in ten technical batches; each included both patients and controls, and all centers. MS analysis was then performed in a blinded manner for each batch.

TABLE 1
Summary of clinical and demographic characteristics
of the study participants.

| | | | Healthy BRCA |
|---|---|---|---|
| | HGOC patients | RRSO | carriers |
| | (n = 26) | (n = 79) | (n = 93) |
| Sampling setting | Operating | Operating | Clinic |
| | room | room | (n = 116) |
| | | | Operating room |
| | | | (n = 1) |
| Age Median (range) | 55.3 | 45.8 | 34.7 |
| | (38-79) | (37-65) | (22-65) |
| Germline mutation | | | |
| BRCA1 (%); | BRCA1- 18 | BRCA1- 30 | BRCA1- 78 |
| | (69%) | (38%) | (66%) |
| BRCA2 (%); | BRCA2- 4 | BRCA2- 23 | BRCA2- 35 |
| | (15.4%) | (29%) | (30%) |
| Other (%) | Other- 4 | Other- 26 | Other- 4 |
| | (15.4%) | (33%) | (3.4%) |
| Stage - FIGO | | N/A | N/A |
| STIC (%) | 1 (4%) | | |
| 1 (%) | 5 (19.2%) | | |
| 2 (%) | 2 (7.7%) | | |
| 3-4 (%) | 18 (69%) | | |
| Prior term | N/A | N/A | 62% |
| pregnancies | | | |
| Number of samples | N/A | N/A | |
| contributed by | | | |
| participant | | | |
| 1 (%) | | | 93 (79.5%) |
| 2 (%) | | | 18 (15.4%) |
| 3 (%) | | | 4 (3.4%) |
| >3 (%) | | | 2 (1.7%) |
| Sample volume: | | | |
| Mean (range) | 4.3 | 3.6 | 4.6 |
| | (1-10) | (1-9) | (1-9) |
| Excluded (%) | 2 (7%) | 21 (26.6%) | 10 (8.5%) |

### UtL Collection

As previously described by Barnabas et al. [14], UtL samples were collected before surgery, after induction of anesthesia, by surgeons in the participating centers. An intrauterine insemination catheter (Insemi™-Cath, Cook Inc. Bloomington, IN, USA) or rigid pipelle uterine sampler (Endosampler, MedGyn, Addison, IL, USA), according to the physician's discretion, was inserted into the endometrial space through the cervical canal without local anesthesia. Ten ml of sterile saline were flushed into the uterine cavity and fallopian tubes and immediately retrieved. The procedure was performed either in a clinic setting (awake) or in an operating room (under general anesthesia).

### Isolation of UtL Microvesicles

The UtL samples were processed and analyzed as previously described [14]. Microvesicle isolation was performed as previously described by Harel et al [Pubmed ID: 25624350, Mol Cell Proteomics 14(4):1127-36 (April 2015)]. Briefly, the UtL samples were immediately centrifuged at 480×g for 15 min to eliminate cells, and supernatants were stored at −80° C. Subsequently, processing and analysis was performed in several batches as follows: 1 ml UtL samples were centrifuged at 1000×g for 20 min to remove cell debris, followed by microvesicle precipitation by centrifugation at 20,000×g for 60 min at 4° C. Pellets were then washed with 1 ml ice-cold PBS and centrifuged again at 20,000×g for 60 min at 4° C.

### In-Solution Digestion and LC-MS/MS Analysis—Proteomics

Microvesicle pellets were solubilized in 8M urea in 100 mM Tris-HCl (pH 8.5), reduced with 1mMdithiothreitol (DTT) at RT for 30 min and alkylated with 5 mM iodoacetamide (IAA) for 30 min in the dark. The lysates were diluted 4-fold with 50 mM ammonium bicarbonate, followed by overnight digestion with Trypsin/Lys-C mix (MS grade Promega, 1:100 enzyme to protein ratio) and sequencing grade modified trypsin (Promega, 1:50 enzyme to protein ratio). Resulting peptides were acidified with trifluoroacetic acid (TFA), purified on CisStageTips (3M Empore™) and vacuum dried. The dried peptides were resuspended in 2% acetonitrile/0.1% TFA prior to the LC-MS/MS analysis.

Peptides were analyzed by liquid-chromatography using the EASY-nLC1000 HPLC (Thermo Fisher Scientific) coupled to the Q-Exactive (QE) Plus or Q-Exactive HF mass spectrometers (Thermo Fisher Scientific, Bremen, Germany). Peptides were separated on 75 μmi.d.×50 cm long EASY-spray PepMap columns (Thermo Fisher Scientific) packed with 2 μm, C18 material with 100 Å pore size. The peptides were loaded with Buffer A (0.1% formic acid) and eluted with a gradient of 7-28% Buffer B (80% acetonitrile/0.1% formic acid), at a flow rate of 300 nl/min, over a gradient of 210 min. MS acquisition was performed in a data-dependent manner, positive-ion mode with selection of the top 10 peptides from each MS spectrum for fragmentation and MS/MS analysis. Full MS spectra were acquired at a resolution of 70,000 (QE-Plus) or 60,000 (QE-HF), m/z range of 300-1800 Th, with AGC target of 3E+06 ions and maximal injection time of 20 ms (QE-Plus) or 100 ms (QE-HF). Peptides were isolated for fragmentation with a window of 1.6 m/z. Higher-energy collisional dissociation (HCD) fragmentation was performed with normalized collisional energy (NCE) of 25 (QE-Plus) or 27 (QE-HF). MS/MS spectra were acquired at a resolution of 17,500 (QE-Plus) or 30,000 (QE-HF), with AGC target of 1E+05 (QE-Plus) or 5E+04 (QE-HF) and maximal injection time of 60 ms (QE-Plus) or 50 ms (QE-HF). Dynamic exclusion was set to 30 sec.

### Experimental Design and Statistical Rationale for Proteomic Analysis

Raw MS files were analyzed in the MaxQuant software (version 1.5.2.18) and the Andromeda search engine. Separate analyses were performed for the training cohort and the validation cohort, using the same parameters. MS/MS spectra were searched against the Uniprot database (version April 2014 with 92,179 entries), a decoy, reverse database of the same size, and a list of common contaminants (245 entries). The peptide search included carbamidomethyl-cysteine as a fixed modification, and N-terminal acetylation and methionine oxidation as variable modifications. MaxQuant search parameters for the initial mass recalibration of the precursors were 20 ppm, and in the main search, the mass tolerance for precursor and fragment ions was 4.5 and 20 ppm, respectively. Trypsin was the specified protease and the maximal number of missed cleavages allowed was two. The minimal peptide length was set to seven amino acids and a minimum of one razor peptide per protein. The search results were filtered with false discovery rate of 0.01 for peptide-spectrum matches and 0.01 for protein identifications. The label-free quantification algorithm (LFQ) in MaxQuant was used for relative quantification, and the ‘match between runs’ feature was enabled. All proteins that could not be discriminated based on the identified peptides were merged into a single protein group.

Initially, proteins identified in the decoy database were filtered out, proteins identified only based on the variable modification site, potential contaminants and immunoglobulins. Bioinformatic analysis of the training cohort was performed on the log 2-LFQ-intensities. Data were filtered to include proteins with valid values in at least 75% of the samples. Missing values were then imputed by replacing them with random, low intensity values that form a normal distribution with a width of 30%, and downshift of 1.8 standard deviations of the general data distribution.

### Statistical Methods

Data were analyzed using SAS® version 9.4 (SAS Institute, Cary NC, USA) software. Descriptive statistics (N, mean, standard deviation, minimum, median and maximum) were calculated for all variables, these are presented in tables by ovarian cancer status. Logistic regression modeling was performed to assess proteomic predictors of ovarian cancer in BRCA carriers and again separately in non-BRCA carriers. Variables were identified and entered into a multivariate model based on several model selection techniques as well as manual statistical evaluation. The variables designated to remain in the model were those factors which remained statistically significant when entered together in the models and maximized the predictive power (AUC of the ROC curve) of the model such that the AUC of the resulting ROC curve was at least 0.95. Several risk scores ranging from 0 to 1 were created from the selected logistic regression models using the model coefficients. The regression model coefficients were used to derive a risk score (termed probability index) as P=e^{Y}/(1+e^{Y}), where Y is the log of the odds of having ovarian cancer and is a linear function of the independent factors.

ROC analysis was performed to test whether the parameters can discriminate between positive and negative subjects. An ROC curve and the area under the ROC curve with 95% confidence interval are presented for each of the chosen models. For each good discriminator, a cut-off value achieving a high sensitivity (100%) and approximately equal sensitivity and specificity will be suggested. For the optimal cut-off values on the good predictors, sensitivity, specificity, PPV, and NPV are presented with 95% confidence intervals.

### Immunohistochemistry

The inventors used their previously reported tissue microarrays (TMAs) for evaluation of the expression of the classifier proteins in FT epithelium and in HGOC tumor sections. Details of the TMAs are published [14]. All slides were simultaneously stained and scored. Primary antibodies used: (A) anti-VPS11 (HPA039020, 1:200, positive control: breast cancer); (B) anti-ATP2B4 (HPA04043, 1:1000, positive control: keratinocytes); (C) anti-CRTAC1 (HPA008175, 1:50, positive control: normal pneumocytes) from Sigma-Aldrich, St. Louis, MO, USA; (D) anti-TMEM67 (13975-1-AP, 1:50, positive control: normal kidney) from ProteinTech, Rosemont, IL, USA.

### Safety Questionnaires

Participants who opted to UtL procedure in a clinic setting, completed a 6-question form assessing: (A) pre-test and (B) post-test anxiety, (C) pain at the time of the procedure and (D) shortly afterwards (up to 30 mins), (E) subjective assessment of length of the procedure, and (F) compliance with additional such procedures in subsequent visits. The scores were graded on a 0-5 scale, ‘0’ reflecting no suffering and ‘5’ representing immense suffering. The performing gynecologist reported the time required to complete the procedure, immediate complications and technical challenges.

### Example 1

### Cohort Characteristics

The inventors established a proteomic diagnostic signature which can be implemented as a decision-support tool for women at high-risk for ovarian cancer due to any previously diagnosed germline mutation/s including but not limited to BRCA1 or BRCA2 or family history of first-degree relative with ovarian cancer. This high-risk population underwent utero-tubal lavage (UtL) and the samples were analyzed as described in the experimental procedures, the risk score was calculated using the models described herein below in Example 3, and the diagnosed women were assigned into one of the following risk groups (a) high-risk (will be referred to surgical consult), (b) low risk (will be re-tested after 6 months), or (c) intermediate risk (will be re-tested every 2-3 months).

UtL procedure is performed by flushing of 10 ml of sterile saline through the cervix into the utero-tubal cavity using a non-proprietary intra-uterine insemination or endometrial sampling catheter. The lavage fluid (which is the LB) is immediately retrieved, normally at a volume of ~5 ml. The procedure can be easily performed in an office setting by experienced gynecologists without formal training, is not time-consuming and does not require special equipment or imaging. Isolation of microparticles from the LB is achieved by centrifugation, followed by solubilization, trypsin digestion and high-resolution mass spectrometric (MS) analysis. This approach enables the identification of thousands of proteins in each LB sample, in a high throughput manner. A label-free quantification algorithm (MaxQuant) provides a relative quantitative measurement of all proteins. Logistic regression models that distinguish patients from controls have been developed and validated. FIG. 1A illustrates the allocation of samples to the various training and validation sets. The main clinical information is summarized in Table 1.

BRCA-mutant control cohort (High risk): From December 2016 to September 2021, 172 asymptomatic women with genetically defined high risk were enrolled, including carriers of germline mutation in BRCA1 (n=89), BRCA2 (n=52) or unspecified gene (n=31) and a total of 197 UtL liquid biopsies were collected. Thirty-two samples were excluded from analysis due to missing information or low protein count. One case was diagnosed with occult malignancy (in situ carcinoma of the fallopian tube, STIC) and the sample was classified as “HGOC”. Of the remaining 164 samples, 106 were collected in a clinic setting and 58 were collected during surgery. The median age of the group was 38 years (range: 22-65). Twenty-four women contributed two or more UtL liquid biopsies on consecutive visits, approximately six months apart.

BRCA-mutant HGOC cohort: Twenty-six HGOC patients with germline BRCA mutation were enrolled and contributed one UtL sample each. In most cases, the germline BRCA mutation was discovered after the diagnosis of HGOC. Two samples were excluded from analysis, resulting in a total of 24 HGOC samples, 6 of which are considered early stage. All high-risk participants (except for one) were of Jewish or European descent.

BRCA-WT cohort: UtL liquid biopsies were collected during gynecological surgeries from HGOC patients and controls with WT or unknown BRCA mutational status. This set included 2 independent cohorts. ‘Validation set 1’ includes 50 HGOC patients and 122 controls, and ‘validation set 2’ includes 50 HGOC patients and 90 controls. This group is further defined as non-carriers or as not having genetic predisposition.

### Example 2

### Proteomic Analysis of UtL Liquid Biopsy of Women at High Risk

To ascertain the similarity of UtL proteomic profile of BRCA1 and BRCA2 carriers, the inventors compared the proteomes of 193 UtL samples of healthy BRCA carriers (mean age 38.0) with the proteomes of 76 UtL of healthy BRCA-WT controls (mean age 40.4). FIG. 1B is a principal component analysis (PCA) indicating that there is no obvious difference between the UtL proteomic content of BRCA1/BRCA2 germline mutation carriers and age matched healthy WT controls. There are also no differences in the overall proteomic profile according to menopausal status (women ≥50 were considered as menopausal and women below 50 as pre-menopausal) in the entire group of healthy participants (FIG. 1C), nor in the subset of BRCA carriers (FIG. 1D). Overall, these data show that the proteomes of UtL liquid biopsies of BRCA carriers are similar across genetic background (BRCA1 and BRCA2 mutations), age and hormonal status. While PCA cannot disclose fine details of differential expression between BRCA mutated and WT tissues, it provides support to the rationale of developing a single assay for detection of HGOC in all BRCA carriers.

### Example 3

### Development of BRCA-Specific Classifiers

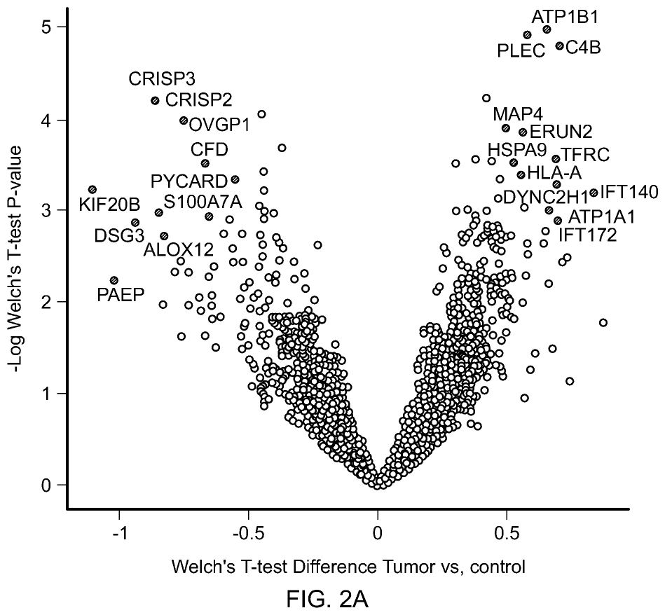

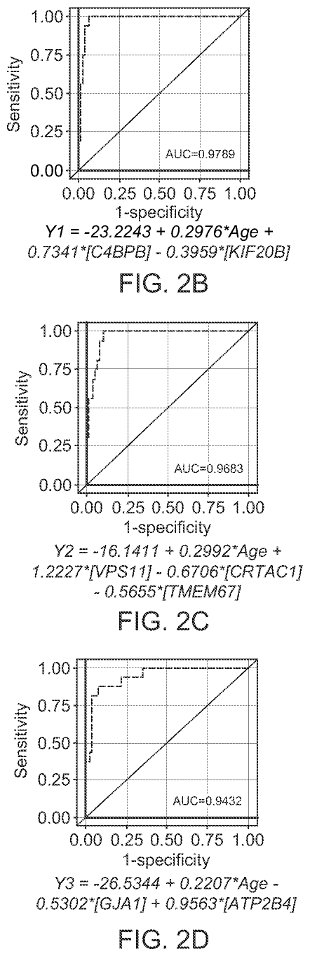

The inventors next compared the proteomes of all UtL liquid biopsies from BRCA-mutant HGOC patients and healthy controls. Combined MS analysis identified a total of 8800 proteins, and an average of 4200 proteins per sample. A volcano scatter plot (FIG. 2A) indicates the significantly different proteins (FDR <0.1) and highlights the significant ones. Several of those are mentioned below as features in the BRCA-specific diagnostic classifier.

The distribution of UtL samples into ‘training’ and ‘validation’ sets is shown in FIG. 1A. Analyses of the training and validation sets, including data normalizations and imputation of missing values, were performed independently. The training set included 110 samples, out of which 90, including 17 HGOC samples and 73 control, with high protein identification rates and complete clinical data were used to develop the diagnostic models. The inventors applied logistic regression tools to define the best models in terms of the AUC and the shape of the ROC curve. Models 1, 2, and 3 are linear functions of the model parameter estimates (see Table 2A-2C, Table 3A-3D and Table 4A-4D, respectively). Using ROC curves (FIGS. 2B, 2B, 2C, 2D) the inventors calculated cut-off scores for 2 operating points (100% sensitivity and mathematical optimum) for each m The models include an age parameter, expressed in years, and the normalized intensity (LFQ) of the following 7 proteins: C4BPB, KIF20B, VPS11, CRTAC1, TMEM67, GJA1, ATP2B4.

$$Model 1 :$$

$$Y 1 = - 23.2243 + 0.2976 * Age + 0.7341 * protein [ C 4 BPB ] - 0.3959 * Protein [ KIF 20 B ] .$$

$$Score Model 1 = exp ( Y 1 ) / ( 1 + exp ( Y 1 ) ) .$$

Model 1 cutoff score for 100% sensitivity is 0.191 and is equal to the optimum cutoff. AUC of the ROC curve is 0.9789.

$$Model 2 :$$

$$Y 2 = - 16.1411 + 0.2992 * Age + 1.2227 * Protein [ VPS 11 ] - 0.6706 * Protein [ CRTAC 1 ] - 0.5655 * Protein [ TMEM 67 ] .$$

$$Score Model 2 = exp ( Y 2 ) / ( 1 + exp ( Y 2 ) ) .$$

Model 2 cutoff for 100% sensitivity is 0.228, and for the optimum is 0.323. AUC is 0.9683.

$$Model 3 :$$

$$Y 3 = - 26.5344 + 0.2207 * Age - 0.5302 * Protein [ GJA 1 ] + 0.9563 * Protein [ ATP 2 B 4 ] .$$

$$Score Model 3 = exp ( Y 3 ) / ( 1 + exp ( Y 3 ) ) .$$

Model 3 cutoff for 100% sensitivity is 0.034, and for the optimum is 0.309. AUC is 0.9432.

The inventors tested the correlation of each of the proteins with age, and found that the correlation is lower than 0.27, although statistically significant in 3 out of 7 proteins (Table 5). This implies that age is not a confounder of the expression of the selected proteins, in the relevant age range. Overall, the sensitivity and negative predictive value (NPV) of each of the three models were 100% which justifies further validation.

Taken together, for patients at genetically defined high risk, including but not limited to germline BRCA1/2 mutation carriers, the three models disclosed herein provide significant signatures that can be used as a decision-support powerful tool for early diagnosis of high-risk ovarian cancer patients.

TABLE 2A
Parameter estimates with standard error, 95%
confidence intervals and p-values of Model 1.

| | | | Standard | Wald | |
|---|---|---|---|---|---|
| Parameter | DF | Estimate | Error | Chi-Square | Pr > ChiSq |
| Intercept | 1 | −23.2243 | 9.2664 | 6.2814 | 0.0122 |
| Age | 1 | 0.2976 | 0.0872 | 11.6624 | 0.0006 |
| C4BPB | 1 | 0.7341 | 0.2503 | 8.6010 | 0.0034 |
| KIF20B | 1 | −0.3959 | 0.1852 | 4.5681 | 0.0326 |

TABLE 2B
Odds ratios with 95% confidence intervals and p-values of Model 1.
Odds Ratio Estimates and Wald Confidence Intervals

| | | | | |
|---|---|---|---|---|
| | | | 95% Confi- | |
| Effect | Unit | Estimate | dence Limits | p-Value |

| | | | | | |
|---|---|---|---|---|---|
| Age | 1.0000 | 1.347 | 1.135 | 1.598 | 0.0006 |
| C4BPB | 1.0000 | 2.084 | 1.276 | 3.403 | 0.0034 |
| KIF20B | 1.0000 | 0.673 | 0.468 | 0.968 | 0.0326 |

TABLE 2C
Sensitivity, Specificity, PPV, and NPV for Model 1 -
optimum and 100% sensitivity operating points.

| | % (n/N) | 95% CI |
|---|---|---|
| Agreement | 93.26% (83/89) | [86.06%; 96.87%] |
| Sensitivity | 100.00% (15/15) | [79.61%; 100.00%] |
| Specificity | 91.89% (68/74) | [83.42%; 96.23%] |
| PPV | 71.43% (15/21) | [50.04%; 86.19%] |
| NPV | 100.00% (68/68) | [94.65%; 100.00%] |

TABLE 3A
Parameter estimates with standard error,
95% confidence intervals and p-values of Model 2.
Analysis of Maximum Likelihood Estimates

| | | | Standard | Wald | Pr > |
|---|---|---|---|---|---|
| Parameter | DF | Estimate | Error | Chi-Square | ChiSq |
| Intercept | 1 | −16.1411 | 5.2234 | 9.5491 | 0.0020 |
| Age | 1 | 0.2992 | 0.0917 | 10.6377 | 0.0011 |
| VPS11 | 1 | 1.2227 | 0.3987 | 9.4053 | 0.0022 |
| CRTAC1 | 1 | −0.6706 | 0.2922 | 5.2682 | 0.0217 |
| TMEM67 | 1 | −0.5655 | 0.2744 | 4.2463 | 0.0393 |

TABLE 3B
Odds ratios with 95% confidence intervals and p-values of Model 2.
Odds Ratio Estimates

| | | |
|---|---|---|
| | Point | 95% Wald |
| Effect | Estimate | Confidence Limits |

| | | | |
|---|---|---|---|
| Age | 1.307 | 1.115 | 1.532 |
| VPS11 | 3.082 | 1.491 | 6.374 |
| CRTAC1 | 0.614 | 0.373 | 1.012 |
| TMEM67 | 0.543 | 0.316 | 0.930 |

TABLE 3C
Sensitivity, Specificity, PPV, and
NPV for Model 2 - 100% sensitivity.

| | % (n/N) | 95% CI |
|---|---|---|
| Agreement | 92.13% (82/89) | [84.64%; 96.14%] |
| Sensitivity | 100.00% (15/15) | [79.61%; 100.00%] |
| Specificity | 90.54% (67/74) | [81.74%; 95.34%] |
| PPV | 68.18% (15/22) | [47.32%; 83.64%] |
| NPV | 100.00% (67/67) | [94.58%; 100.00%] |

TABLE 3D
Sensitivity, Specificity, PPV, and NPV
for Model 2 - optimum operating points.

| | % (n/N) | 95% CI |
|---|---|---|
| Agreement | 93.26% (83/89) | [86.06%; 96.87%] |
| Sensitivity | 93.33% (14/15) | [70.18%; 98.81%] |
| Specificity | 93.24% (69/74) | [85.14%; 97.08%] |
| PPV | 73.68% (14/19) | [51.21%; 88.19%] |
| NPV | 98.57% (69/70) | [92.34%; 99.75%] |

TABLE 4A
Parameter estimates with standard error,
95% confidence intervals and p-values of Model 3.
Analysis of Maximum Likelihood Estimates

| | | | Standard | Wald | Pr > |
|---|---|---|---|---|---|
| Parameter | DF | Estimate | Error | Chi-Square | ChiSq |
| Intercept | 1 | −26.5344 | 7.2149 | 13.5254 | 0.0002 |
| Age | 1 | 0.2207 | 0.0588 | 14.0825 | 0.0002 |
| GJA1 | 1 | −0.5302 | 0.2336 | 5.1529 | 0.0232 |
| ATP2B4 | 1 | 0.9563 | 0.3316 | 8.3182 | 0.0039 |

TABLE 4B
Odds ratios with 95% confidence intervals and p-values of Model 3.
Odds Ratio Estimates and Wald Confidence Intervals

| | | | | |
|---|---|---|---|---|
| | | | 95% Confi- | |
| Effect | Unit | Estimate | dence Limits | p-Value |

| | | | | | |
|---|---|---|---|---|---|
| Age | 1.0000 | 1.247 | 1.111 | 1.399 | 0.0002 |
| GJA1 | 1.0000 | 0.588 | 0.372 | 0.930 | 0.0232 |
| ATP2B4 | 1.0000 | 2.602 | 1.359 | 4.983 | 0.0039 |

TABLE 4C
Sensitivity, Specificity, PPV, and
NPV for Model 3 - 100% sensitivity.

| | % (n/N) | 95% CI |
|---|---|---|
| Agreement | 73.03% (65/89) | [63.00%; 81.16%] |
| Sensitivity | 100.00% (15/15) | [79.61%; 100.00%] |
| Specificity | 67.57% (50/74) | [56.27%; 77.14%] |
| PPV | 38.46% (15/39) | [24.89%; 54.10%] |
| NPV | 100.00% (50/50) | [92.87%; 100.00%] |

TABLE 4D
Sensitivity, Specificity, PPV, and NPV
for Model 3 - optimum operating points.

| | % (n/N) | 95% CI |
|---|---|---|
| Agreement | 89.89% (80/89) | [81.89%; 94.59%] |
| Sensitivity | 86.67% (13/15) | [62.12%; 96.26%] |
| Specificity | 90.54% (67/74) | [81.74%; 95.34%] |
| PPV | 65.00% (13/20) | [43.29%; 81.88%] |
| NPV | 97.10% (67/69) | [90.03%; 99.20%] |

TABLE 5
Correlation between patient's age and Mass spectrometry measurement
of classifier proteins, in the BRCA mutant population.

| | | | Lower 95% | Lower 95% | |
|---|---|---|---|---|---|
| Protein | | | Confidence | Confidence | |
| name | N | Correlation | Limit | Limit | P-Value |
| GJA1 | 93 | 0.22 | 0.01 | 0.40 | 0.0363 |
| C4BPB | 93 | 0.16 | −0.04 | 0.35 | 0.1192 |
| ATP2B4 | 93 | 0.25 | 0.05 | 0.43 | 0.0147 |
| KIF20B | 93 | −0.07 | −0.27 | 0.13 | 0.4926 |
| VPS11 | 93 | 0.22 | 0.02 | 0.41 | 0.0323 |
| CRTAC1 | 93 | 0.27 | 0.06 | 0.44 | 0.0099 |
| TMEM67 | 93 | 0.18 | −0.02 | 0.37 | 0.0762 |

### Example 4

### Diagnostic Signature for Average Risk

The inventors next established a proteomic diagnostic signature which can be applied as a decision-support tool for women at average-risk for ovarian cancer who are not carriers of any cancer-predisposing germline mutation. Postmenopausal women are subjected to such test as a screening assay, or in case of suspicious gynecological finding, as recommended by their treating physician.

The average risk discovery cohort included 172 subjects who were not identified as BRCA carriers (either negative for founder mutations or upon full gene sequencing or unknown status). This cohort included 50 positives for ovarian cancer. The validation set for the average risk diagnostic included 62 subjects, 25 of which were positive for ovarian cancer. Samples were prepared as described in Example 1.

One model that provided good discriminatory power, in terms of the AUC and the shape of the ROC curve (providing the required optimal points on the curve) was chosen and is presented in this section.

$$Model 4 parameters :$$

$$Age ( categorical ) + Cathepsin D ( CTSD ) + Spectrin beta chain , non - erythrocytic 2 ( SPTBN 2 ) + Arfaptin - 1 ( ARFIP 1 ) + Transport and Golgi organization protein 1 homolog ( MIA 3 ) + CD 109 antigen ( CD 109 ) + Immunity - related GTPase family Q protein ( IRGQ ) + Golgi phosphoprotein 3 ( GOLPH 3 )$$

The formula for risk calculation includes an age factor in patients at age 55 or greater.

The following Formula was used:

$${Y}_{4} = - 7.1661 + ( if 55 \leq Age < 65 then 1 ) * 2.5801 + ( if 65 \leq Age then 1 ) * 4.137 + Protein CTSD * 0.7246 + Protein SPTBN 2 * 0.301 - Protein ARFIP 1 * 0.5111 + Protein MIA 3 * 0.2578 - Protein CD 109 * 0.7254 + Protein IRGQ * 0.5501 - Protein GOLPH 3 * 0.5006$$

$$Score Model 4 = exp ( {Y}_{4} ) / ( 1 + exp ( {Y}_{4} ) ) .$$

Model 4 cutoff for 100% sensitivity is 0.091, and for the optimum is 0.263.

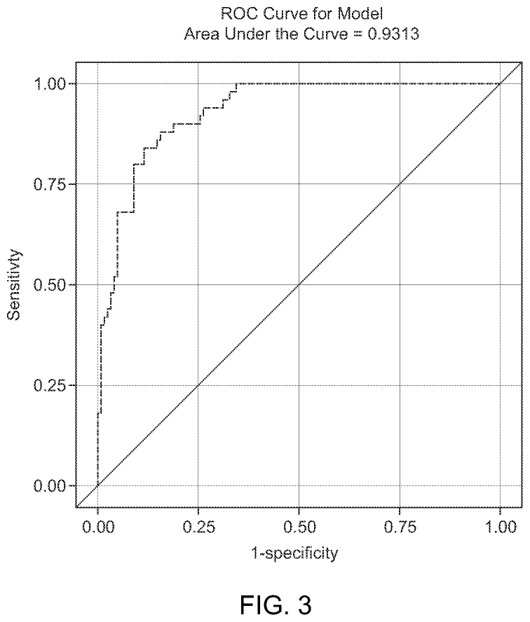

FIG. 3 shows the ROC curve of Model 4, that is also presented in Table 6A, the Odds ratio is shown in Table 6B, the discriminatory parameters for 100% sensitivity cutoff is shown in Table 6C and optimal point is shown in Table 6D.

Model 4 therefore establishes the feasibility of using the CTSD+SPTBN2, ARFIP1, MIA3, CD109, IRGQ, GOLPH3 proteins as markers for detecting high-risk ovarian patients.

The numeric values that are included in the calculations are the normalized intensity values (e.g., label-free quantification LFQ intensity).

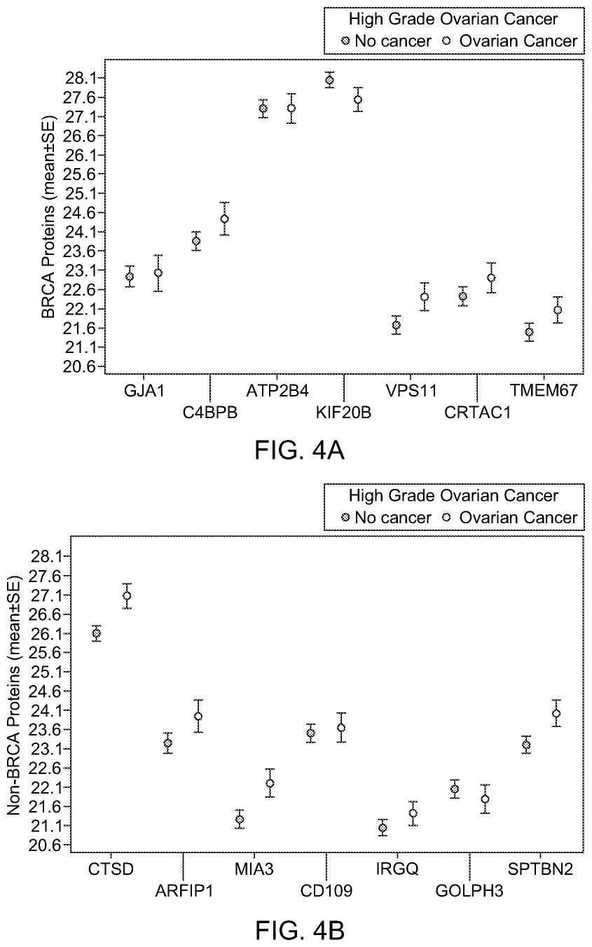

FIG. 4A and FIG. 4B, present example for the expression rate of the various biomarkers in UtL samples.

FIG. 4A shows that in samples taken from carriers of at least one mutation in BRCA1 and/or BRCA2, any one of the GJA1, C4BPB, VPS11, CRTAC1, TMEM67 biomarker proteins display increased expression (upregulated genes), in samples of carrier patients diagnosed with ovarian cancer, whereas the KIF20B biomarker shows reduced expression in ovarian cancer samples (down regulation).

FIG. 4B shows that in samples taken from non-carriers, the CTSD, ARFIP1, MIA3, CD109, IRGQ, SPTBN2 biomarker proteins display increased expression (upregulated genes), in samples of ovarian cancer, whereas GOLPH3 shows reduced expression in ovarian cancer samples (down regulation).

TABLE 6A
Parameter estimates with standard error, 95% confidence intervals and p-values of Model 4.
Analysis of Maximum Likelihood Estimates

| | | | Standard | Wald | | |
|---|---|---|---|---|---|---|
| Parameter | DF | Estimate | Error | Chi-Square | Pr > ChiSq | Label |
| Intercept | 1 | −7.1661 | 2.7467 | 6.8070 | 0.0091 | Intercept: Ocancer = Ovarian Cancer |
| 55 ≤ Age < 65 | 1 | 2.5801 | 0.6681 | 14.9134 | 0.0001 | Age Group |
| 65 ≤ Age | | 4.1370 | 0.8503 | 23.6731 | <.0001 | Age Group |
| CTSD | 1 | 0.7246 | 0.2202 | 10.8241 | 0.0010 | A0A1B0GVD5; A0A1B0GWE8; P07339; A0A1B0GW44; |
| | | | | | | A0A1B0GVP3; A0A1B0GV23; A0A1B0GU03; C9JH19; |
| | | | | | | H7C469; F8WD96; F8W787; A0A1B0GV01 |
| SPTBN2 | 1 | 0.3010 | 0.1460 | 4.2500 | 0.0392 | O15020; A4QPE4; E9PJZ2 |
| ARFIP1 | 1 | −0.5111 | 0.1478 | 11.9668 | 0.0005 | P53367 |
| MIA3 | 1 | 0.2578 | 0.1284 | 4.0349 | 0.0446 | Q5JRA6; A0A0A0MRH6 |
| CD109 | 1 | −0.7254 | 0.1605 | 20.4306 | <.0001 | Q6YHK3 |
| IRGQ | 1 | 0.5501 | 0.1923 | 8.1855 | 0.0042 | Q8WZA9; M0QZP8 |
| GOLPH3 | 1 | −0.5006 | 0.1450 | 11.9134 | 0.0006 | Q9H4A6; D6REM1 |

TABLE 6B
Odds ratios with 95% confidence intervals and p-values of Model 4.
Odds Ratio Estimates and Wald Confidence Intervals

| | | | 95% Confi- | | |
|---|---|---|---|---|---|
| Effect | Unit | Estimate | dence Limits | | p-Value |
| 55 ≤ | 1.0000 | 13.199 | 3.563 | 48.891 | 0.0001 |
| Age < 65 | | | | | |
| 65 ≤ Age | 1.0000 | 62.613 | 11.828 | 331.448 | <.0001 |
| CTSD | 1.0000 | 2.064 | 1.340 | 3.178 | 0.0010 |
| SPTBN2 | 1.0000 | 1.351 | 1.015 | 1.799 | 0.0392 |
| ARFIP1 | 1.0000 | 0.600 | 0.449 | 0.801 | 0.0005 |
| MIA3 | 1.0000 | 1.294 | 1.006 | 1.664 | 0.0446 |
| CD109 | 1.0000 | 0.484 | 0.353 | 0.663 | <.0001 |
| IRGQ | 1.0000 | 1.733 | 1.189 | 2.527 | 0.0042 |
| GOLPH3 | 1.0000 | 0.606 | 0.456 | 0.805 | 0.0006 |

TABLE 6C
Sensitivity, Specificity, PPV, and
NPV for Model 4 - 100% sensitivity.

| | % (n/N) | 95% CI |
|---|---|---|
| Agreement | 75.58% (130/172) | [68.65%; 81.40%] |
| Sensitivity | 100.00% (50/50) | [92.87%; 100.00%] |
| Specificity | 65.57% (80/122) | [56.78%; 73.41%] |
| PPV | 54.35% (50/92) | [44.20%; 64.15%] |
| NPV | 100.00% (80/80) | [95.42%; 100.00%] |

TABLE 6D
Sensitivity, Specificity, PPV, and NPV
for Model 4 - optimum operating points.

| | % (n/N) | 95% CI |
|---|---|---|
| Agreement | 85.47% (147/172) | [79.42%; 89.96%] |
| Sensitivity | 88.00% (44/50) | [76.20%; 94.38%] |
| Specificity | 84.43% (103/122) | [76.95%; 89.80%] |
| PPV | 69.84% (44/63) | [57.64%; 79.76%] |
| NPV | 94.50% (103/109) | [88.51%; 97.45%] |

### Example 5

### Validation of the Classifiers

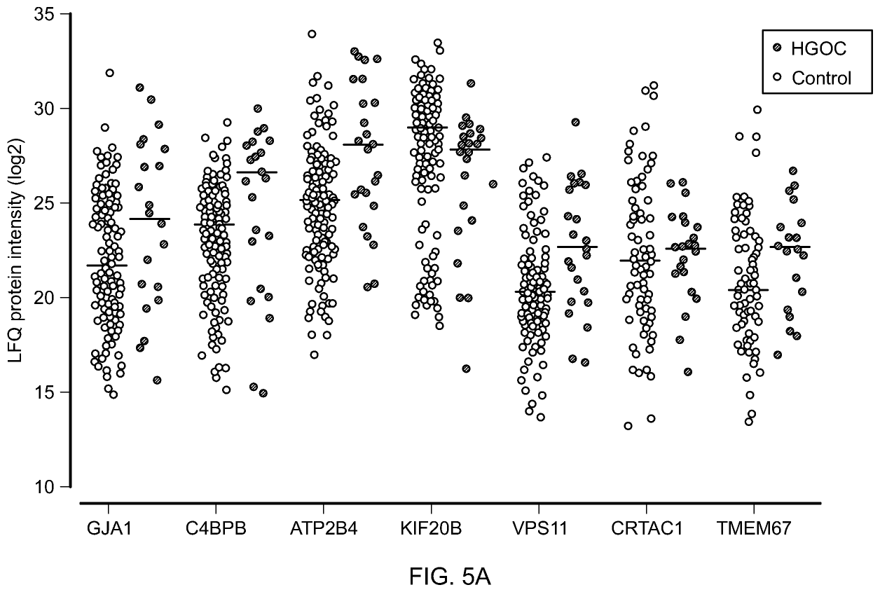

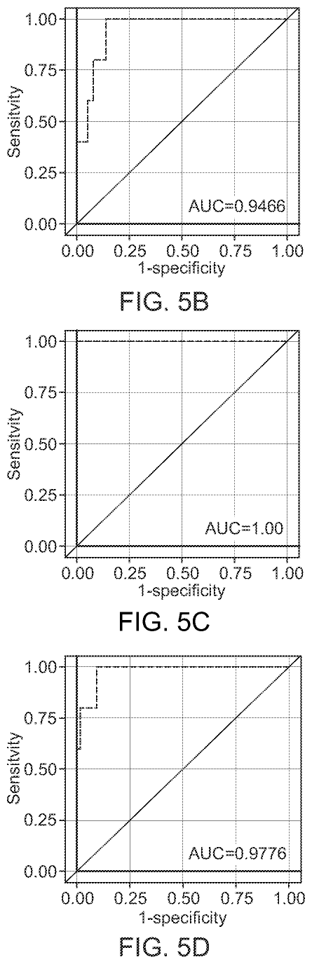

An independent validation set composed of 112 samples, of which 14 were excluded, resulting in 7 HGOC UtL samples (all of them were BRCA1 mutant) and 91 control samples, was similarly processed, analyzed, and normalized. Risk scores of the 3 models were calculated. The results of sensitivity, specificity, PPV and NPV are seen in Table 7. Intensities of the seven proteins in all BRCA-mutated UtL of HGOC patients and controls are plotted in FIG. 5A). ROC curves of the independent validation sets are in FIGS. 5B, 5C, 5D, with AUC >0.94 for all models.

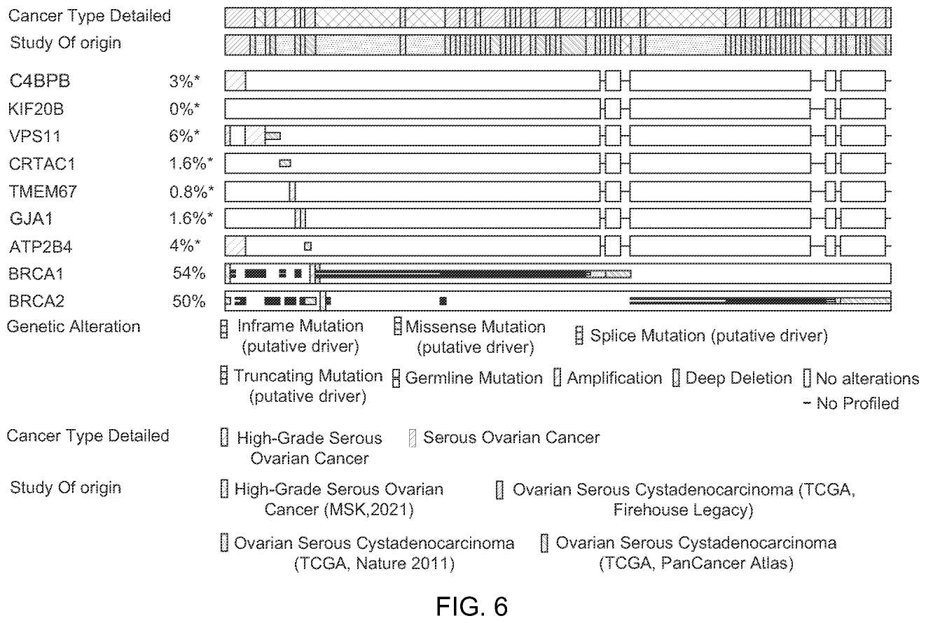

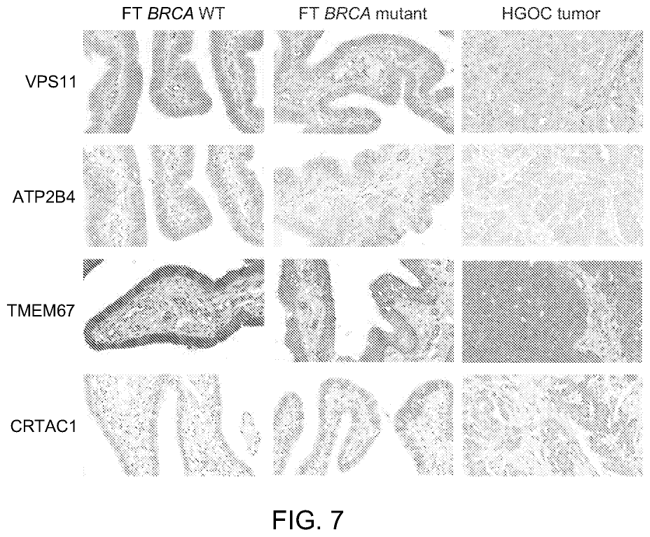

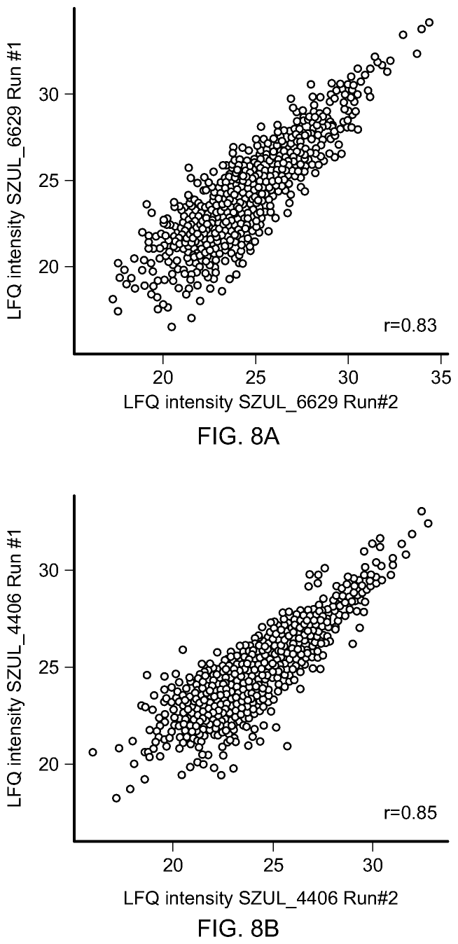

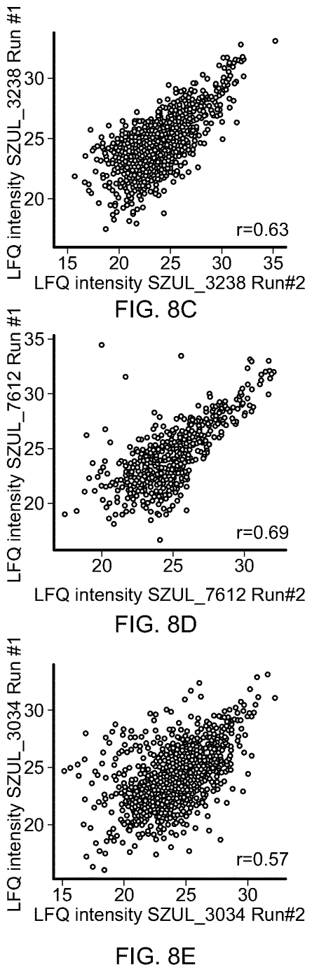

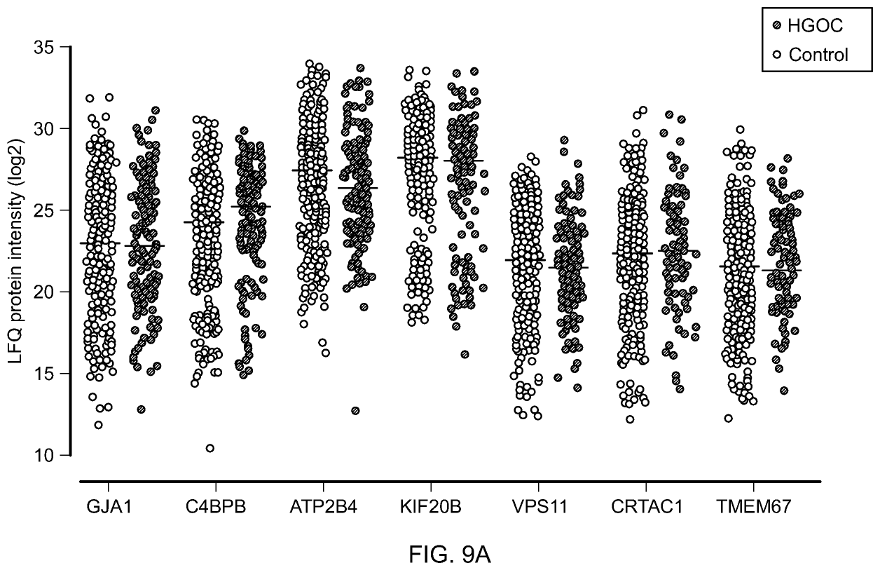

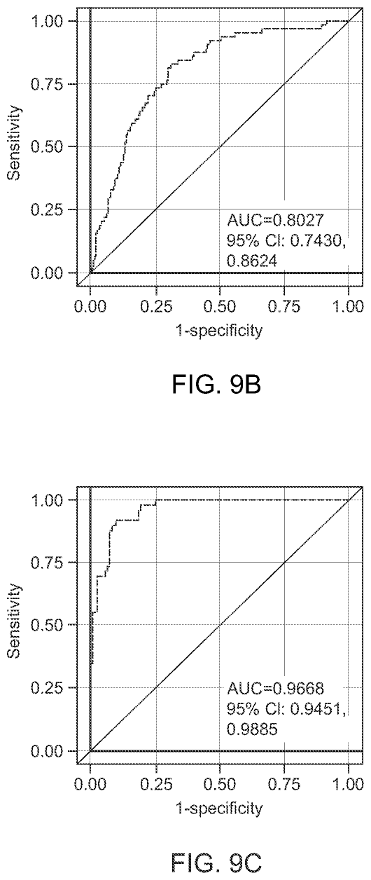

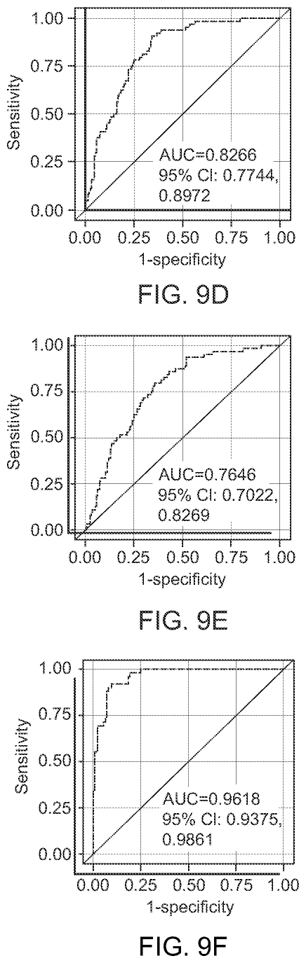

The levels of the proteins GJA1, C4BPB, ATP2B4, VPS11 and TMEM67 are significantly higher in UtL liquid biopsies from BRCA-mutant HGOC patients compared to controls. The level of KIF20B was lower in HGOC patients' samples compared to controls. The TCGA HGOC database was explored using cBioPortal (Gao J, et al. Sci Signal. 2013 Apr. 2; 6(269); Cerami E, et al. Cancer Discov. 2012 May; 2(5):401-4) for BRCA1 and BRCA2 mutated samples (n=133 across 4 studies) and confirmed that altered expression of the seven proteins does not correlate with BRCA mutant status (FIG. 6). Next, in order to further investigate the expression of the seven proteins that compose the classifier the inventors performed immunohistochemical staining of sections of tissue microarrays (TMAs) representing the following clinical-histological groups: (A) HGOC tumors; (B) fimbriae of asymptomatic BRCA mutation carriers, removed during RRSO; (C) grossly normal fimbriae of women with HGOC, BRCA WT. These TMAs were previously described in detail [14]. Commercially available monoclonal antibodies targeting the proteins: VPS11, ATP2B4, TMEM67, CRTAC1 were used. Attempts to obtain reliable immunohistochemical stainings for the three other proteins are in process. Representative images are seen in FIG. 7. For these four proteins we detected differential expression between FT epithelium of BRCA-WT and BRCA mutant women. The intensity of the staining of VPS11, ATP2B4 and TMEM in HGOC tumors was comparable to that seen in normal FT of BRCA mutant women.

Nineteen healthy BRCA carriers were followed longitudinally the high-risk clinic at Sheba Medical Center, Israel, with UtL performed every 6 months, up to 5 times (Table 4, the samples are labeled with a letter a-d following their serial number). All the liquid biopsies from these women were scored as “normal”, suggesting biological reproducibility which is essential for clinical implementation. Furthermore, several UtL samples were run twice in different batches, about 9 months apart, and on different MS systems to determine the technical reproducibility of the proteomic assay and the stability of the UtL samples after suboptimal storage conditions. Representative correlation plots are presented in FIGS. 8A, 8B, 8C, 8D, 8E. Overall, these results suggest degree of reproducibility that is consistent with real-world limitations.

The intensity values of the seven proteins that construct the classifiers were compared between 163 UtL liquid biopsies of HGOC patients and 433 control samples (including ones that were previously excluded), regardless of their BRCA status (FIG. 9A). The intensities of all the proteins except C4BPB were not significantly different in UtL liquid biopsies from HGOC patients compared to controls, when BRCA status was ignored. Next, the inventors tested the discriminant performance of Models 1-3 on two sets of UtL liquid biopsies of participants who have WT BRCA or an unknown BRCA status. Of note, in the Israeli population, the chance of a HGOC patient to be a BRCA mutation carrier is ~30%. Eighty percent of HGOC patients had undergone basic genetic testing for common founder mutations, rather than full gene sequencing, and a negative results indicates a residual risk of <8% for having a pathogenic mutation (Bernstein-Molho R, et als. Breast Cancer Res Treat. 2020 Jun. 1; 181(2):445-53). The two independent cohorts were normalized and analyzed separately and included HGOC patients (n=50 and 50, respectively), and healthy controls (n=122 and 90, respectively). Table 8 shows the sensitivity, specificity, PPV and NPV of the 3 models in these cohorts. Risk score calculations for Model 2 are missing in set 2, since not all the proteins were detected. The ROC curves in FIGS. 9B, 9C, 9D, 9E, 9F indicate inconsistencies between the two sets, which warrant further validation. Overall, while the classifier's performance is worse for the WT population than the BRCA mutation carriers, it is still better than any other available diagnostic tool in a non-BRCA population as well. Importantly, the classifier also identified correctly one of two HGOC cases of women who were BRCA WT but had a mutation in a mismatch repair gene compatible with diagnosis of Lynch Syndrome.

TABLE 7
Sensitivity, specificity, PPV and NPV of the validation set of Models 1-3, both optimum and 100% sensitivity operating points.
Model 2 risk score cannot be calculated for most of the validation set, since not all the proteins were detected.

| | | | Model 2 - 100% | | Model 2 - | | Model 3 - 100% | | Model 3 - | |
|---|---|---|---|---|---|---|---|---|---|---|
| | Model 1 | | sensitivity | | optimum | | sensitivity | | optimum | |
| | % | 95% | % | 95% | % | 95% | % | 95% | % | 95% |
| | (n/N) | CI | (n/N) | CI | (n/N) | CI | (n/N) | CI | (n/N) | CI |
| Overall | 84.00% | [75.58%; | 66.67% | [30.00%; | 66.67% | [30.00%; | 87.63% | [79.61%; | 94.85% | [88.50%; |
| Accuracy | (84/100) | 89.90%] | (4/6) | 90.32%] | (4/6) | 90.32%] | (85/97) | 92.78%] | (92/97) | 97.78%] |
| Sensitivity | 71.43% | [35.89%; | 50.00% | [15.00%; | 50.00% | [15.00%; | 66.67% | [30.00%; | 50.00% | [18.76%; |
| | (5/7) | 91.78%] | (2/4) | 85.00%] | (2/4) | 85.00%] | (4/6) | 90.32%] | (3/6) | 81.24%] |
| Specificity | 84.95% | [76.30%; | 100.00% | [34.24%; | 100.00% | [34.24%; | 89.01% | [80.94%; | 97.80% | [92.34%; |
| | (79/93) | 90.82%] | (2/2) | 100.00%] | (2/2) | 100.00%] | (81/91) | 93.92%] | (89/91) | 99.40%] |
| PPV | 26.32% | [11.81%; | 100.00% | [34.24%; | 100.00% | [34.24%; | 28.57% | [11.72%; | 60.00% | [23.07%; |
| | (5/19) | 48.79%] | (2/2) | 100.00%] | (2/2) | 100.00%] | (4/14) | 54.65%] | (3/5) | 88.24%] |
| NPV | 97.53% | [91.44%; | 50.00% | [15.00%; | 50.00% | [15.00%; | 97.59% | [91.63%; | 96.74% | [90.85%; |
| | (79/81) | 99.32%] | (2/4) | 85.00%] | (2/4) | 85.00%] | (81/83) | 99.34%] | (89/92) | 98.88%] |

TABLE 8
Sensitivity, specificity, PPV and NPV of Models 1-3, both optimum and 100% sensitivity operating
points, on BRCA WT and unknown BRCA status UtL samples. Model 2 risk score cannot be calculated
for part of the validation set, since not all the proteins were detected.

| | | | Model 2 - 100% | | Model 2 - | | Model 3 - 100% | | Model 3 - | |
|---|---|---|---|---|---|---|---|---|---|---|
| | Model 1 | | sensitivity | | optimum | | sensitivity | | optimum | |
| | % | 95% | % | 95% | % | 95% | % | 95% | % | 95% |
| | (n/N) | CI | (n/N) | CI | (n/N) | CI | (n/N) | CI | (n/N) | CI |
| Set 1 | | | | | | | | | | |
| Overall | 53.49% | [46.04%; | 58.14% | [50.67%; | 61.63% | [54.18%; | 44.77% | [37.53%; | 57.56% | [50.09%; |
| Accuracy | (92/172) | 60.78%] | (100/172) | 65.26%] | (106/172) | 68.57%] | (77/172) | 52.23%] | (99/172) | 64.70%] |
| Sensitivity | 80.00% | [66.96%; | 92.00% | [81.16%; | 88.00% | [76.20%; | 92.00% | [81.16%; | 74.00% | [60.45%; |
| | (40/50) | 88.76%] | (46/50) | 96.85%] | (44/50) | 94.38%] | (46/50) | 96.85%] | (37/50) | 84.13%] |
| Specificity | 42.62% | [34.20%; | 44.26% | [35.76%; | 50.82% | [42.06%; | 25.41% | [18.52%; | 50.82% | [42.06%; |
| | (52/122) | 51.49%] | (54/122) | 53.12%] | (62/122) | 59.53%] | (31/122) | 33.80%] | (62/122) | 59.53%] |
| PPV | 36.36% | [27.98%; | 40.35% | [31.80%; | 42.31% | [33.25%; | 33.58% | [26.21%; | 38.14% | [29.10%; |
| | (40/110) | 45.67%] | (46/114) | 49.53%] | (44/104) | 51.91%] | (46/137) | 41.84%] | (37/97) | 48.09%] |
| NPV | 83.87% | [72.79%; | 93.10% | [83.57%; | 91.18% | [82.06%; | 88.57% | [74.05%; | 82.67% | [72.57%; |
| | (52/62) | 91.00%] | (54/58) | 97.29%] | (62/68) | 95.89%] | (31/35) | 95.46%] | (62/75) | 89.58%] |
| Set 2 | | | | | | | | | | |
| Overall | 76.38% | [68.28%; | 40.00% | [19.82%; | 46.67% | [24.81%; | 77.12% | [68.76%; | 77.97% | [69.67%; |
| Accuracy | (97/127) | 82.92%] | (6/15) | 64.25%] | (7/15) | 69.88%] | (91/118) | 83.77%] | (92/118) | 84.50%] |
| Sensitivity | 93.33% | [82.14%; | 66.67% | [20.77%; | 66.67% | [20.77%; | 88.89% | [76.50%; | 66.67% | [52.07%; |
| | (42/45) | 97.71%] | (2/3) | 93.85%] | (2/3) | 93.85%] | (40/45) | 95.16%] | (30/45) | 78.64%] |
| Specificity | 67.07% | [56.34%; | 33.33% | [13.81%; | 41.67% | [19.33%; | 69.86% | [58.56%; | 84.93% | [75.00%; |
| | (55/82) | 76.28%] | (4/12) | 60.94%] | (5/12) | 68.05%] | (51/73) | 79.18%] | (62/73) | 91.37%] |
| PPV | 60.87% | [49.07%; | 20.00% | [5.67%; | 22.22% | [6.32%; | 64.52% | [52.08%; | 73.17% | [58.07%; |
| | (42/69) | 71.52%] | (2/10) | 50.98%] | (2/9) | 54.74%] | (40/62) | 75.26%] | (30/41) | 84.31%] |
| NPV | 94.83% | [85.86%; | 80.00% | [37.55%; | 83.33% | [43.65%; | 91.07% | [80.74%; | 80.52% | [70.31%; |
| | (55/58) | 98.23%] | (4/5) | 96.38%] | (5/6) | 96.99%] | (51/56) | 96.13%] | (62/77) | 87.82%] |

### Example 6

### Feasibility and Safety

In order to establish UtL as an acceptable office procedure for monitoring young high-risk population of women, it has to be technically feasible to all gynecologists, cheap, simple, and safe. To assess these aspects, 18 gynecologists with varied level of skills and experience from 3 different Israeli medical centers participated in the study and acquired UtL liquid biopsies in a clinic setting. The average volume was 4.2 mL, and of the 137 liquid biopsies, 14 (10%) had a volume of less than 1 mL, which was arbitrarily defined as the volume used in the proteomic assay. The majority of samples with insufficient volume were taken by inexperienced residents.

We collected 137 questionnaires reporting pain and stress scores, as well as their attitude towards undergoing the same procedure in subsequent follow-up visits. The median pain score was 2, ranging from ‘0’ representing no pain (n=30), to ‘5’ representing excruciating pain (n=4). Local anesthesia or cervical dilatation were not allowed in this trial. The only factor which significantly correlated with the pain score is parity and only nulliparous women reported intense pain (score 4-5; Table 9).

Adverse events included prolonged pelvic pain (n=2), vaginal discharge (n=1), and vaginal bleeding starting the next day (n=1), all of which did not require intervention and the severity of all adverse events was defined as Grade 1. 105 women consented to additional sampling in the future, and none withdrew consent or expressed refusal to undergo the procedure in future visits. This real-life data suggests that the UtL technique is both highly safe and feasible.

TABLE 9
Factors associated with the pain scores reported by participants
undergoing UtL procedure. “High pain score” means
3-5 on a 0-5 scale. “Low pain score” means 0-2 on
a 0-5 scale. P-value was calculated using t-test between
the groups of the continuous variables and chi-square test
between the groups of the attribute's variables.

Pain Score

| | | High Pain | Low Pain | |
|---|---|---|---|---|
| | | Score | Score | P-Value |
| Performing gynecologist | | | | |
| Resident | N | 8 | 22 | 0.8736 |
| | % | 22.86 | 21.57 | |
| Senior | N | 27 | 80 | |
| | % | 77.14 | 78.43 | |
| Duration of | N | 33 | 85 | 0.5533 |
| procedure | Mean | 4.79 | 4.48 | |
| (minute) | SD | 2.759 | 2.471 | |
| | Median | 5.00 | 5.00 | |
| Sample | N | 32 | 98 | 0.8847 |
| volume (mL) | Mean | 4.41 | 4.34 | |
| | SD | 2.010 | 2.338 | |
| Age at | N | 35 | 102 | 0.2685 |
| sampling | Mean | 34.11 | 35.19 | |
| | SD | 4.529 | 5.052 | |
| | Median | 34.00 | 35.00 | |
| Previous delivery | | | | |
| No | N | 18 | 30 | 0.0159 |
| | % | 52.94 | 30.00 | |
| Yes | N | 16 | 70 | |
| | % | 47.06 | 70.00 | |
| Stress Level | | | | |
| High | N | 5 | 5 | 0.1316 |
| Stress Level | % | 14.29 | 4.90 | |
| Low | N | 29 | 95 | |
| Stress Level | % | 82.86 | 93.14 | |

1.-18. (canceled)

19. A diagnostic composition comprising at least two detecting molecules or any combination or mixture of plurality of detecting molecules specific for determining the level of expression of at least two biomarker protein/s, wherein said at least two biomarker protein/s comprise at least one of:
(i) at least one of C4b-binding protein beta chain (C4BPB) and Kinesin-like protein Family Member 20B (KIF20B); (ii) at least one of Vacuolar protein sorting-associated protein 11 homolog (VPS11), Cartilage acidic protein 1 (CRTAC1) and Meckelin (TMEM67); (iii) at least one of Gap junction alpha-1 protein (GJA1) and Plasma membrane calcium-transporting ATPase 4 (ATP2B4); and (iv) any combination of at least two biomarker proteins of any one of (i), (ii) and (iii) protein/s; and
wherein each of said detecting molecules is specific for one of said biomarker protein/s.

20. The composition according to claim 19, wherein said composition comprises at least four detecting molecules or any combination or mixture of plurality of detecting molecules specific for determining the level of expression of at least four of:
C4BPB, KIF20B, VPS11, CRTAC1, TMEM67, GJA1 and ATP2B4 protein/s or any combination thereof, and wherein each of said detecting molecules is specific for one of said biomarker proteins.

21. The composition according to claim 19, wherein said composition comprises detecting molecules or any combination or mixture of plurality of detecting molecules specific for determining the level of expression of C4BPB, KIF20B, VPS11, CRTAC1, TMEM67, GJA1 and ATP2B4 protein/s; and wherein each of said detecting molecules is specific for one of said biomarker proteins.

22. The composition according to claim 19, wherein said detecting molecules are selected from amino acid detecting molecules and nucleic acid detecting molecules, or any combinations thereof; and wherein at least one of:
(I) wherein said amino acid detecting molecules comprise at least one of:
a. at least two labeled or tagged protein/s or any fragment/s, peptide/s or mixture/s thereof, of at least one of: (i) at least one of C4BPB and KIF20B: (ii) at least one of VPS11, CRTAC1 and TMEM67: (iii) at least one of GJA1 and ATP2B4; and (iv) any combination of at least two biomarker proteins of any one of (i), (ii) and (iii), protein/s or any fragment/s, peptide/s or mixture/s thereof;
b. at least one antibody specific for said at least two of said biomarker protein/s:
c. at least one peptide aptamer/s specific for at least one of said at least two biomarker protein/s; and
d. any combination of (a), (b) and (c); and
(II) wherein said nucleic acid detecting molecules comprise at least one of:
a. at least one nucleic acid aptamer/s, each aptamer is specific for at least one of said at least two biomarker proteins; and
b. at least one oligonucleotide/s, each oligonucleotide specifically hybridizes to a nucleic acid sequence encoding at least one of said at least two biomarker protein/s.

23.-24. (canceled)

25. The composition according to claim 22, wherein:
(a) said detecting molecules are attached to a solid support; or
(b) said detecting molecules are provided in a mixture.

26. (canceled)

27. A kit comprising:
a. at least two detecting molecules specific for determining the level of expression of at least two biomarker protein/s in a biological sample, wherein said at least two biomarker protein/s comprise at least one of: (i) at least one of C4BPB and KIF20B; (ii) at least one of VPS11, CRTAC1 and TMEM67; (iii) at least one of GJA1 and ATP2B4; and (iv) any combination of at least two biomarker proteins of any one of (i), (ii) and (iii) protein/s or any combination thereof;
wherein each of said detecting molecule/s is specific for one of said biomarker proteins; said kit optionally further comprises at least one of:
b. pre-determined calibration curve/s or predetermined standard/s providing standard expression values of said at least two biomarker/s; and
c. at least one control sample.

28. The kit according to claim 27, comprising:
a. at least two detecting molecules specific for determining the level of expression of at least four biomarker protein/s in a biological sample, wherein said at least four biomarker protein/s are selected from at least one of: (i) at least one of C4BPB and KIF20B; (ii) at least one of VPS11, CRTAC1 and TMEM67; (iii) at least one of GJA1 and ATP2B4; and (iv) any combination of at least four biomarker proteins of any one of (i), (ii) and (iii) protein/s or any combination thereof,
wherein each of said detecting molecule/s is specific for one of said biomarker proteins; said kit optionally further comprises at least one of:
b. pre-determined calibration curve/s or predetermined standard/s providing standard expression values of said at least two biomarker/s; and
c. at least one control sample.

29. The kit according to claim 27, comprising:
a. at least one detecting molecule specific for determining the level of expression of C4BPB, KIF20B, VPS11, CRTAC1, TMEM67, GJA1 and ATP2B4 protein/s or any combination thereof, wherein each of said detecting molecule/s is specific for one of said biomarker proteins; said kit optionally further comprises at least one of:
b. pre-determined calibration curve/s or predetermined standard/s providing standard expression values of said biomarker protein/s; and
c. at least one control sample.

30. The kit according to claim 27, wherein said detecting molecules are selected from amino acid detecting molecule/s, nucleic acid detecting molecule/s, and any combinations thereof; and wherein at least one of:
(I) wherein said amino acid detecting molecules comprise at least one of:
a. at least two labeled or tagged protein/s or any fragment/s, peptide/s or mixture/s thereof, of at least one of: (i) at least one of C4BPB and KIF20B: (ii) at least one of VPS11, CRTAC1 and TMEM67: (iii) at least one of GJA1 and ATP2B4; and (iv) any combination of at least two biomarker proteins of any one of (i), (ii) and (iii), protein/s or any fragment/s, peptide/s or mixture/s thereof;
b. at least one antibody specific for said at least two of said biomarker protein/s;
c. at least one peptide aptamer/s specific for at least one of said at least two biomarker protein/s; and
d. any combination of (a), (b) and (c); and
(II) wherein said nucleic acid detecting molecules comprise at least one of:
a. at least one nucleic acid aptamer/s, each aptamer is specific for at least one of said at least two biomarker proteins; and
b. at least one oligonucleotide/s, each oligonucleotide specifically hybridizes to a nucleic acid sequence encoding at least one of said at least two biomarker protein/s.

31.-32. (canceled)

33. The kit according to claim 27, wherein:
(a) said detecting molecule/s are attached to a solid support; or
(b) said detecting molecule/s is provided in a mixture.

34. (canceled)

35. The kit according to claim 27, further comprising instructions for use, wherein said instructions comprise at least one of:
a. instructions for carrying out the detection and quantification of the expression of said at least two biomarker proteins of at least one of: (i) at least one of C4BPB and KIF20B; (ii) at least one of VPS11, CRTAC1 and TMEM67; (iii) at least one of GJA1 and ATP2B4; and (iv) any combination of at least two biomarker proteins of any one of (i), (ii) and (iii) biomarker protein/s;
and optionally, of a control reference protein; and
b. instructions for determining if the expression values of at least two of said biomarker protein/s is positive or negative with respect to a corresponding predetermined standard expression value or with expression value of at least one of said at least two biomarker protein/s in said at least one control sample.

36. The kit according to claim 27, further comprising at least one of:
(a) at least one reagent for conducting a mass spectrometry assay;
(b) at least one reagent for conducting an immunological assay; and
(c) at least one device or means for obtaining a body fluid sample and for isolating microvesicles from said body fluid sample.

37.-38. (canceled)

39. The kit according to claim 27, for use in a method for detecting ovarian cancer in a subject, and/or for use in a method of early detection of High-grade ovarian carcinoma, the method comprising:
(a) determining the expression level of at least two biomarker proteins in at least one biological sample of said subject, to obtain an expression value for each of said at least two biomarker protein/s, wherein said at least two biomarker proteins comprise at least one of: (i) at least one of C4b-binding protein beta chain (C4BPB) and Kinesin-like protein Family Member 20B (KIF20B); (ii) at least one of Vacuolar protein sorting-associated protein 11 homolog (VPS11), Cartilage acidic protein 1 (CRTAC1) and Meckelin (TMEM67); and (iii) at least one of Gap junction alpha-1 protein (GJA1) and Plasma membrane calcium-transporting ATPase 4 (ATP2B4); (iv) any combination of at least two biomarker proteins of any one of (i), (ii) and (iii); optionally, the subject is a subject determined as a carrier for at least one mutation in at least one gene associated with high risk for ovarian cancer; and
(b) determining if the expression value obtained in step (a) for each of said at least two biomarker protein/s is positive or negative with respect to a predetermined standard expression value or to an expression value of said biomarker protein/s in at least one control sample;
wherein at least one of:
a positive expression value of at least two biomarker proteins of at least one of said (i) C4BPB; (ii) at least one of VPS11, CRTAC1 and TMEM67; (iii) at least one of GJA1 and ATP2B4 and/or a negative expression value of KIF20B; and (iv) any combination of at least two biomarker proteins of any one of (i), (ii) and (iii), biomarker protein/s in said sample, indicates that said subject is suffering from ovarian cancer; optionally, wherein said subject is a subject determined as a carrier for at least one mutation in at least one gene associated with high risk for ovarian cancer, and wherein said at least one gene associated with high risk for ovarian cancer is at least one of Breast cancer type 1 susceptibility protein (BRCA1) and/or Breast cancer ty e 2 susceptibility protein (BRCA2) and/or said ovarian cancer is High-grade ovarian carcinoma.

40.-41. (canceled)

42. The kit according to claim 27, wherein at least one of:
(a) said sample is a body fluid sample,
(b) said sample is microvesicles prepared from said body fluid;
(c) said body fluid is at least one of uterine lavage fluid (UtL) and plasma; and
(d) said sample comprises microvesicles isolated from UtL.

43.-45. (canceled)

46. A method for treating and/or ameliorating ovarian cancer in a subject in need thereof, the method comprising:
(a) detecting ovarian cancer in said subject by the steps of:
(I) determining the expression level of at least two biomarker proteins in at least one biological sample of the diagnosed subject, to obtain an expression value for each of said at least two biomarker protein/s, wherein said at least two biomarker protein/s comprise at least one of: (i) at least one of C4b-binding protein beta chain (C4BPB) and Kinesin-like protein Family Member 20B (KIF20B); (ii) at least one of Vacuolar protein sorting-associated protein 11 homolog (VPS11), Cartilage acidic protein 1 (CRTAC1) and Meckelin (TMEM67); and (iii) at least one of Gap junction alpha-1 protein (GJA1) and Plasma membrane calcium-transporting ATPase 4 (ATP2B4); (iv) any combination of at least two biomarker proteins of any one of (i), (ii) and (iii); optionally, the subject is a subject determined as a carrier for at least one mutation in at least one gene associated with high risk for ovarian cancer;
(II) determining if the expression value obtained in step (I) for each of the at least two biomarker protein/s is positive or negative with respect to a predetermined standard expression value or to an expression value of said biomarker protein/s in at least one control sample;
wherein at least one of:
a positive expression value of at least two biomarker proteins of at least one of said (i) C4BPB; (ii) at least one of VPS11, CRTAC1 and TMEM67; (iii) at least one of GJA1 and ATP2B4 and/or a negative expression value of KIF20B; and (iv) any combination of at least two biomarker proteins of any one of (i), (ii) and (iii), biomarker protein/s in said sample, indicates that said subject is suffering from ovarian cancer; and
(b) administering to a subject detected as suffering from ovarian cancer according to step (a), an effective amount of at least one anti-cancer agent, or subjecting said subject to an anti cancer treatment regimen.

47. The method according to claim 46, wherein said subject is a subject determined as a carrier for at least one mutation in at least one gene associated with high risk for ovarian cancer, and wherein said at least one gene associated with high risk for ovarian cancer is at least one of Breast cancer type I susceptibility protein (BRCA1) and/or Breast cancer type 2 susceptibility protein (BRCA2).

48. The method according to claim 46, wherein said ovarian cancer is High-grade ovarian carcinoma.

49. The method according to claim 46, wherein determining the level of expression of at least two of said biomarker protein/s is performed by the step of contacting at least one detecting molecule, or any combination or mixture of plurality of detecting molecules, with a biological sample of said subject, or with any protein or nucleic acid product obtained therefrom, wherein each of said detecting molecules is specific for one of said biomarker proteins.

50. The method according to claim 46, wherein said detecting molecules are selected from amino acid detecting molecule/s, nucleic acid detecting molecule/s, and any combinations thereof; and
wherein at least one of:
(I) wherein said amino acid detecting molecules comprise at least one of:
a. at least two labeled or tagged protein/s or any fragment/s, peptide/s or mixture/s thereof, of at least one of: (i) at least one of C4BPB and KIF20B; (ii) at least one of VPS11, CRTAC1 and TMEM67; (iii) at least one of GJA1 and ATP2B4; and (iv) any combination of at least two biomarker proteins of any one of (i), (ii) and (iii), protein/s or any fragment/s, peptide/s or mixture/s thereof,
b. at least one antibody specific for said at least two of said biomarker protein/s;
c. at least one peptide aptamer/s specific for at least one of said at least two biomarker protein/s; and
d. any combination of (a), (b) and (c); and
(II) wherein said nucleic acid detecting molecules comprise at least one of:
a. at least one nucleic acid aptamer/s, each aptamer is specific for at least one of said at least two biomarker proteins; and
b. at least one oligonucleotide/s, each oligonucleotide specifically hybridizes to a nucleic acid sequence encoding at least one of said at least two biomarker protein/s.

51. A diagnostic method for detecting ovarian cancer in a subject, by using the diagnostic composition according to claim 19, the method comprising:
(a) determining the expression level of at least two biomarker proteins in at least one biological sample of said subject, to obtain an expression value for each of said at least two biomarker protein/s, wherein said at least two biomarker proteins comprise at least one of: (i) at least one of C4b-binding protein beta chain (C4BPB) and Kinesin-like protein Family Member 2(B (KIF20B); (ii) at least one of Vacuolar protein sorting-associated protein 11 homolog (VPS11), Cartilage acidic protein 1 (CRTAC1) and Meckelin (TMEM67); and (iii) at least one of Gap junction alpha-1 protein (GJA1) and Plasma membrane calcium-transporting ATPase 4 (ATP2B4); (iv) any combination of at least two biomarker proteins of any one of (i), (ii) and (iii); optionally, the subject is a subject determined as a carrier for at least one mutation in at least one gene associated with high risk for ovarian cancer; and
(b) determining if the expression value obtained in step (a) for each of said at least two biomarker protein/s is positive or negative with respect to a predetermined standard expression value or to an expression value of said biomarker protein/s in at least one control sample;
wherein at least one of:
a positive expression value of at least two biomarker proteins of at least one of said (i) C4BPB; (ii) at least one of VPS11, CRTAC1 and TMEM67; (iii) at least one of GJA1 and ATP2B4 and/or a negative expression value of KIF20B; and (iv) any combination of at least two biomarker proteins of any one of (i), (ii) and (iii), biomarker protein/s in said sample, indicates that said subject is suffering from ovarian cancer;
optionally, wherein said subject is a subject determined as a carrier for at least one mutation in at least one gene associated with high risk for ovarian cancer, and wherein said at least one gene associated with high risk for ovarian cancer is at least one of Breast cancer type I susceptibility protein (BRCA1) and/or Breast cancer type 2 susceptibility protein (BRCA2).
